(19) United States
(12) Patent Application Publication
(10) Pub. No.: US 2026/0308577 A1
(43) Pub. Date: Oct. 8, 2026

(54) MODIFIED GUIDE RNAS

(71) Applicant: Genentech, Inc., South San Francisco, CA (US)

(72) Inventors: Stefan George Koenig, South San Francisco, CA (US); Ke Zhang, South San Francisco, CA (US); Alexandre Pierre Goyon, South San Francisco, CA (US)

(73) Assignee: Genentech, Inc., South San Francisco, CA (US)

(21) Appl. No.: 19/343,854

(22) Filed: Sep. 29, 2025

Related U.S. Application Data
(63) Continuation of application No. PCT/US24/23575, filed on Apr. 8, 2024.
(60) Provisional application No. 63/458,017, filed on Apr. 7, 2023.

(51) Int. Cl. C12N 15/11; C12N 9/22; C12N 13/00; C12N 15/87; C12N 15/90

(52) U.S. Cl. CPC C12N 15/11; C12N 9/226; C12N 13/00; C12N 15/87; C12N 15/907; C12N 2310/20; C12N 2310/321; C12N 2310/322; C12N 2310/3231

(57) ABSTRACT

The present disclosure relates to guide RNAs that have been modified to improve performance and stability. In certain embodiments, a guide RNA of the present disclosure includes one or more modifications at its 5′ terminus and/or one or more modifications at its 3′ terminus.

### CROSS-REFERENCE TO RELATED APPLICATIONS

The application is a continuation of International Patent Application No. PCT/US24/23575, filed Apr. 8, 2024, which claims priority to U.S. Provisional Application No. 63/458,017, filed Apr. 7, 2023, the contents of which are incorporated herein by reference in their entireties.

### SEQUENCE LISTING

The specification further incorporates by reference the Sequence Listing submitted electronically on Jun. 12, 2026. Pursuant to 37 C.F.R. § 1.52(e)(5), the Sequence Listing file, identified as P38397-US-1_SeqListing.xml, is 337,786 bytes and was created on Jun. 12, 2026. The Sequence Listing does not extend beyond the scope of the specification and thus does not contain new matter.

### FIELD

The present disclosure relates to guide RNAs that have been modified to improve stability and improve performance.

### BACKGROUND

Clusters of regularly interspaced short palindromic repeats (CRISPR)/CRISPR-associated protein (Cas) systems are widely used to edit the genomes of various cell types. CRISPR/Cas systems can be categorized into two classes (class I and class II), which can be further subdivided into at least six different types: Type I, Type II, Type III, Type IV, Type V and Type VI. In a Type II CRISPR/Cas system, a Cas protein (such as Cas9) forms a complex with a guide RNA (gRNA) molecule and binds to a target nucleic acid that has a protospacer adjacent motif (PAM) and a spacer. The gRNA molecule includes a sequence that is complementary to the spacer in the target nucleic acid and functions to guide the Cas protein to the target nucleic acid. The recognition and binding of the target nucleotide by the Cas protein:gRNA complex induces cleavage of the target nucleic acid.

It has been found that gRNA molecules can be degraded in cells by nuclease cleavage, e.g., endonuclease or exonuclease cleavage, which can affect the efficacy of a CRISPR-Cas system. Therefore, there is a need in the art for gRNA molecules with improved stability and improved gene editing efficiency.

### SUMMARY

The present disclosure provides modified guide RNA molecules. In certain embodiments, the guide RNA molecule includes (i) a phosphorodithioate linkage between the first and second nucleotides at the 5′ terminus of the guide RNA molecule, (ii) a phosphorodithioate linkage between the 3′ terminal (“n”) nucleotide and the n−1 nucleotide of the guide RNA molecule or (iii) a phosphorodithioate linkage between the first and second nucleotides at the 5′ terminus of the guide RNA molecule and a phosphorodithioate linkage between the n and n−1 nucleotides of the guide RNA molecule.

In certain embodiments, the guide RNA molecule further includes phosphorodithioate linkages between the second and third nucleotides and the third and fourth nucleotides at the 5′ terminus of the guide RNA molecule. In certain embodiments, the guide RNA molecule includes phosphorodithioate linkages between the first and second nucleotides, the second and third nucleotides, and the third and fourth nucleotides at the 5′ terminus of the guide RNA molecule. In certain embodiments, the guide RNA molecule includes phosphorodithioate linkages between the first and second nucleotides, the second and third nucleotides, the third and fourth nucleotides, and the fourth and fifth nucleotides at the 5′ terminus of the guide RNA molecule.

In certain embodiments, the guide RNA molecule comprises phosphorodithioate linkages between the n and n−1 nucleotides and the n−1 and n−2 nucleotides or the n and n−1 nucleotides and the n−2 and n−3 nucleotides of the guide RNA molecule. In certain embodiments, the guide RNA molecule comprises phosphorodithioate linkages between the n and the n−1 nucleotides, the n−1 and n−2 nucleotides, and the n−2 and n−3 nucleotides of the guide RNA molecule. In certain embodiments, the guide RNA molecule comprises phosphorodithioate linkages between the n and n−1 nucleotides, the n−1 and n−2 nucleotides, the n−2 and n−3 nucleotides, and the n−3 and n−4 nucleotides of the guide RNA molecule.

In certain embodiments, the first nucleotide at 5′ terminus of the guide RNA molecule includes a modification selected from the group consisting of (a) a 2′-fluoro modified nucleotide, (b) a 2′-O-methyl modified nucleotide, (c) a 2′-O-(2-methoxyethyl) modified nucleotide, (d) a locked nucleic acid (LNA), (e) a deoxyribose nucleotide and a combination of two or more of (a)-(e). In certain embodiments, the first nucleotide at 5′ terminus of the guide RNA molecule comprises a 2′-fluoro modified nucleotide. In certain embodiments, the first nucleotide at 5′ terminus of the guide RNA molecule comprises a 2′-O-methyl modified nucleotide. In certain embodiments, the first nucleotide at 5′ terminus of the guide RNA molecule comprises a 2′-O-(2-methoxyethyl) modified nucleotide. In certain embodiments, the first nucleotide at 5′ terminus of the guide RNA molecule comprises an LNA. In certain embodiments, the first nucleotide at 5′ terminus of the guide RNA molecule comprises a deoxyribose nucleotide.

In certain embodiments, (i) the second nucleotide, (ii) the third nucleotide, (iii) the fourth nucleotide, (iv) the second and third nucleotides, (v) the second and fourth nucleotides, (vi) the third and fourth nucleotides or (v) the second, third and fourth nucleotides at the 5′ terminus of the guide RNA molecule includes a modification selected from the group consisting of (a) a 2′-fluoro modified nucleotide, (b) a 2′-O-methyl modified nucleotide, (c) a 2′-O-(2-methoxyethyl) modified nucleotide, (d) a locked nucleic acid (LNA), (e) a deoxyribose nucleotide; and a combination of two or more of (a)-(e).

In certain embodiments, the guide RNA molecule includes three consecutive 2′-fluoro modified nucleotides at the first three nucleotides at the 5′ terminus of the guide RNA molecule. In certain embodiments, the guide RNA molecule includes three consecutive 2′-O-methyl modified nucleotides at the first three nucleotides at the 5′ terminus of the guide RNA molecule. In certain embodiments, the guide RNA molecule includes four consecutive 2′-O-methyl modified nucleotides at the first four nucleotides at the 5′ terminus of the guide RNA molecule. In certain embodiments, the guide RNA molecule includes three consecutive 2′-O-(2-methoxyethyl) modified nucleotides at the first three nucleotides at the 5′ terminus of the guide RNA molecule. In certain embodiments, the guide RNA molecule includes three consecutive LNAs at the first three nucleotides at the 5′ terminus of the guide RNA molecule. In certain embodiments, the guide RNA molecule includes three consecutive deoxyribose nucleotides at the first three nucleotides at the 5′ terminus of the guide RNA molecule.

In certain embodiments, the 3′ terminal (“n”) nucleotide of the guide RNA molecule includes a modification selected from the group consisting of (a) a 2′-fluoro modified nucleotide, (b) a 2′-O-methyl modified nucleotide, (c) a 2′-O-(2-methoxyethyl) modified nucleotide, (d) a locked nucleic acid (LNA), (e) a deoxyribose nucleotide and a combination of two or more of (a)-(e). In certain embodiments, the n nucleotide of the guide RNA molecule comprises a 2′-fluoro modified nucleotide. In certain embodiments, the n nucleotide of the guide RNA molecule comprises a 2′-O-methyl modified nucleotide. In certain embodiments, the n nucleotide of the guide RNA molecule comprises a 2′-O-(2-methoxyethyl) modified nucleotide. In certain embodiments, the n nucleotide of the guide RNA molecule comprises an LNA. In certain embodiments, the n nucleotide of the guide RNA molecule comprises a deoxyribose nucleotide.

In certain embodiments, (i) the n−1 nucleotide, (ii) the n−2 nucleotide, (iii) the n−3 nucleotide, (iv) the n−4 nucleotide, (v) the n and n−1 nucleotides, (vi) the n and n−2 nucleotides, (vii) the n−1 and n−2 nucleotides, (viii) the n, n−1 and n−2 nucleotides, (ix) the n−1, n−2 and n−3 nucleotides, (x) the n, n−1, n−2 and n−3 nucleotides, or (xi) the n, n−1, n−2, n−3 and n−4 nucleotides at the 3′ terminus of the guide RNA molecule each comprise a modification selected from the group consisting of (a) a 2′-fluoro modified nucleotide, (b) a 2′-O-methyl modified nucleotide, (c) a 2′-O-(2-methoxyethyl) modified nucleotide, (d) a locked nucleic acid (LNA), (e) a deoxyribose nucleotide and a combination of two or more of (a)-(e). In certain embodiments, the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are 2′-fluoro modified nucleotides. In certain embodiments, the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are 2′-O-methyl modified nucleotides. In certain embodiments, the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are 2′-O-(2-methoxyethyl) modified nucleotides. In certain embodiments, the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are LNAs. In certain embodiments, the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are deoxyribose nucleotides.

The present disclosure further provides nucleic acids that include a polynucleotide encoding a guide RNA molecule disclosed herein. In certain embodiments, the nucleic acid further includes a polynucleotide encoding an RNA-guided nuclease. In certain embodiments, the RNA-guided nuclease is a Cas protein. In certain embodiments, the Cas protein is selected from the group consisting of a Cas9, a Cas12, a Cas13 and a combination thereof. The present disclosure provides vectors that include a nucleic acid disclosed herein.

The present disclosure provides compositions that include a guide RNA molecule disclosed herein. In certain embodiments, a composition of the present disclosure includes a vector disclosed herein. In certain embodiments, the composition further includes an RNA-guided nuclease. Alternatively or additionally, the composition further includes a nucleic acid encoding the RNA-guided nuclease. In certain embodiments, the RNA-guided nuclease is a Cas protein, e.g., a Cas9, a Cas12 and/or a Cas13. In certain embodiments, the composition includes a nucleic acid disclosed herein, e.g., a nucleic acid that includes a polynucleotide encoding a guide RNA molecule.

The present disclosure further includes a ribonucleoprotein (RNP) complex that includes a modified guide RNA molecule of the present disclosure and an RNA-guided nuclease. In certain embodiments, the RNA-guided nuclease is a Cas protein. In certain embodiments, the Cas protein is selected from the group consisting of a Cas9, a Cas12, a Cas13 and a combination thereof.

The present disclosure further includes a cell comprising a composition or an RNP complex disclosed herein.

The present disclosure further provides a method of modifying a cell. In certain embodiments, the method includes contacting the cell with a composition or an RNP complex of disclosed herein. In certain embodiments, said contacting includes introducing the composition into the cell by electroporation.

The present disclosure provides methods of treating a subject in need thereof. In certain embodiments, the method includes modifying a cell of the subject ex vivo by contacting the cell with a composition or an RNP complex disclosed herein and returning the modified cell to the subject.

The present disclosure further provides gene editing systems that comprise one or more modified guide RNAs of the present disclosure, one or more nucleic acids, one or more vectors, one or more compositions and/or one or more RNP complexes disclosed herein.

### BRIEF DESCRIPTION OF THE FIGURES

FIG. 1 provides an exemplary structure of a gRNA of the present disclosure.

FIG. 2 provides elution profiles of exemplary modified gRNAs of the present disclosure.

FIG. 3 provides elution profiles of exemplary modified gRNAs of the present disclosure.

FIG. 4 provides elution profiles of exemplary modified gRNAs of the present disclosure.

FIG. 5 provides elution profiles of exemplary modified gRNAs of the present disclosure.

FIG. 6 shows the editing efficiency of exemplary modified gRNAs of the present disclosure at different concentrations compared to reference gRNAs.

FIG. 7 shows the editing efficiency of exemplary modified gRNAs of the present disclosure at different concentrations.

FIG. 8 shows the editing efficiency of exemplary modified gRNAs of the present disclosure compared to reference gRNAs using cells from different donors.

FIG. 9 shows the editing efficiency of exemplary modified gRNAs of the present disclosure at different concentrations compared to a reference gRNA.

FIG. 10 shows the editing efficiency of exemplary modified gRNAs of the present disclosure at different concentrations compared to reference gRNAs.

FIG. 11 shows the editing efficiency of exemplary modified gRNAs of the present disclosure compared to a reference gRNA.

FIG. 12 shows the editing efficiency of exemplary modified gRNAs of the present disclosure compared to reference gRNAs.

FIG. 13 provides the percent cell viability and total number of cells in the presence of exemplary modified gRNAs of the present disclosure under knock-out conditions compared to a reference gRNA.

FIG. 14 provides the percent cell viability and total number of cells in the presence of exemplary modified gRNAs of the present disclosure under knock-out conditions compared to reference gRNAs.

FIG. 15 provides the percent cell viability and total number of cells in the presence of exemplary modified gRNAs of the present disclosure under knock-in conditions compared to a reference gRNA.

FIG. 16 provides the percent cell viability and total number of cells in the presence of exemplary modified gRNAs of the present disclosure under knock-out conditions compared to reference gRNAs.

FIG. 17 provides elution profiles of exemplary modified gRNAs of the present disclosure.

FIG. 18 provides elution profiles of reference gRNA 3 under basic stress (pH 11), acidic stress (pH 5) and oxidative stress (0.3% H₂O₂) conditions.

FIG. 19 provides elution profiles of reference gRNA 3 under basic stress (pH 11), acidic stress (pH 5) and oxidative stress (0.3% H₂O₂) conditions over certain time periods.

FIG. 20 provides elution profiles of exemplary modified gRNAs of the present disclosure under acidic stress (pH 5) conditions.

FIG. 21 provides elution profiles of exemplary modified gRNAs of the present disclosure under acidic stress (pH 5) conditions.

FIG. 22 provides elution profiles of exemplary modified gRNAs of the present disclosure under acidic stress (pH 5) conditions.

FIG. 23 provides elution profiles of exemplary modified gRNAs of the present disclosure under acidic stress (pH 5) conditions.

FIG. 24 provides elution profiles of exemplary modified gRNAs of the present disclosure under basic stress (pH 11) conditions.

FIG. 25 provides elution profiles of exemplary modified gRNAs of the present disclosure under basic stress (pH 11) conditions.

FIG. 26 provides elution profiles of exemplary modified gRNAs of the present disclosure under basic stress (pH 11) conditions.

FIG. 27 provides elution profiles of exemplary modified gRNAs of the present disclosure under basic stress (pH 11) conditions.

FIG. 28 provides elution profiles of exemplary modified gRNAs of the present disclosure under thermal stress.

FIG. 29 provides elution profiles of exemplary modified gRNAs of the present disclosure under thermal stress.

FIG. 30 provides elution profiles of exemplary modified gRNAs of the present disclosure under thermal stress.

FIG. 31 provides elution profiles of exemplary modified gRNAs of the present disclosure under thermal stress.

FIG. 32 provides elution profiles of exemplary modified gRNAs of the present disclosure under oxidative stress (0.3% H₂O₂).

FIG. 33 provides elution profiles of exemplary modified gRNAs of the present disclosure under oxidative stress (0.3% H₂O₂).

FIG. 34 provides elution profiles of exemplary modified gRNAs of the present disclosure under oxidative stress (0.3% H₂O₂).

FIG. 35 provides elution profiles of exemplary modified gRNAs of the present disclosure under oxidative stress (0.3% H₂O₂).

FIG. 36 provides elution profiles of exemplary modified gRNAs of the present disclosure under oxidative stress (0.3% H₂O₂) over a 5-day period.

FIG. 37 provides the changes in purity of exemplary modified gRNAs of the present disclosure over a 3-day time period under acidic stress (pH 5) conditions.

FIG. 38 provides the changes in purity of exemplary modified gRNAs of the present disclosure over a 24-hour time period under basic stress (pH 11).

FIG. 39 provides the changes in purity of exemplary modified gRNAs of the present disclosure over a 7-day time period under thermal stress.

FIG. 40 provides the changes in purity of exemplary modified gRNAs of the present disclosure over a 5-day time period under oxidative stress (0.3% H₂O₂).

FIG. 41 provides elution profiles of exemplary modified gRNAs of the present disclosure.

FIG. 42 provides elution profiles of exemplary modified gRNAs of the present disclosure under acidic stress (pH 5) conditions.

FIG. 43 provides elution profiles of exemplary modified gRNAs of the present disclosure under acidic stress (pH 5) conditions.

FIG. 44 provides elution profiles of exemplary modified gRNAs of the present disclosure under acidic stress (pH 5) conditions.

FIG. 45 provides elution profiles of exemplary modified gRNAs of the present disclosure under acidic stress (pH 5) conditions.

FIG. 46 provides elution profiles of exemplary modified gRNAs of the present disclosure under basic stress (pH 11) conditions.

FIG. 47 provides elution profiles of exemplary modified gRNAs of the present disclosure under basic stress (pH 11) conditions.

FIG. 48 provides elution profiles of exemplary modified gRNAs of the present disclosure under basic stress (pH 11) conditions.

FIG. 49 provides elution profiles of exemplary modified gRNAs of the present disclosure under basic stress (pH 11) conditions.

FIG. 50 provides elution profiles of exemplary modified gRNAs of the present disclosure under thermal stress.

FIG. 51 provides elution profiles of exemplary modified gRNAs of the present disclosure under thermal stress.

FIG. 52 provides elution profiles of exemplary modified gRNAs of the present disclosure under thermal stress.

FIG. 53 provides elution profiles of exemplary modified gRNAs of the present disclosure under thermal stress.

FIG. 54 provides elution profiles of exemplary modified gRNAs of the present disclosure under oxidative stress (0.3% H₂O₂).

FIG. 55 provides elution profiles of exemplary modified gRNAs of the present disclosure under oxidative stress (0.3% H₂O₂).

FIG. 56 provides the changes in purity of exemplary modified gRNAs of the present disclosure over a 7-day time period under thermal stress.

FIG. 57 provides the changes in purity of exemplary modified gRNAs of the present disclosure under acidic stress (pH 5) conditions.

FIG. 58 provides the changes in purity of exemplary modified gRNAs of the present disclosure under basic stress (pH 11) conditions.

FIG. 59 provides the changes in purity of exemplary modified gRNAs of the present disclosure under oxidative stress (0.3% H₂O₂).

FIG. 60 shows the editing efficiency of exemplary modified gRNAs of the present disclosure at a mid-timepoint compared to a reference gRNA.

FIG. 61 shows the editing efficiency of exemplary modified gRNAs of the present disclosure at the end timepoint compared to a reference gRNA.

FIG. 62 shows the cell expansion of the cells of donor 1 at day 5 and from day 5 to day 12 after transfection (Tfx) with the exemplary modified gRNAs of the present disclosure.

FIG. 63 shows the phenotype of exemplary modified gRNAs of the present disclosure at the end timepoint compared to a reference gRNA.

FIG. 64 shows the editing efficiency of exemplary modified gRNAs of the present disclosure at a mid-timepoint compared to a reference gRNA.

FIG. 65 shows the editing efficiency of exemplary modified gRNAs of the present disclosure at the end timepoint compared to a reference gRNA.

FIG. 66 shows the cell expansion of the cells of donor 2 at day 5 and from day 5 to day 12 after transfection (Tfx) with the exemplary modified gRNAs of the present disclosure.

FIG. 67 shows the phenotype of exemplary modified gRNAs of the present disclosure at the end timepoint compared to a reference gRNA.

FIG. 68 shows the editing efficiency of exemplary modified gRNAs of the present disclosure at a mid-timepoint compared to a reference gRNA.

FIG. 69 shows the editing efficiency of exemplary modified gRNAs of the present disclosure at the end timepoint compared to a reference gRNA.

FIG. 70 shows the cell expansion of the cells of donor 3 at day 5 and from day 5 to day 12 after transfection (Tfx) with the exemplary modified gRNAs of the present disclosure.

### DETAILED DESCRIPTION

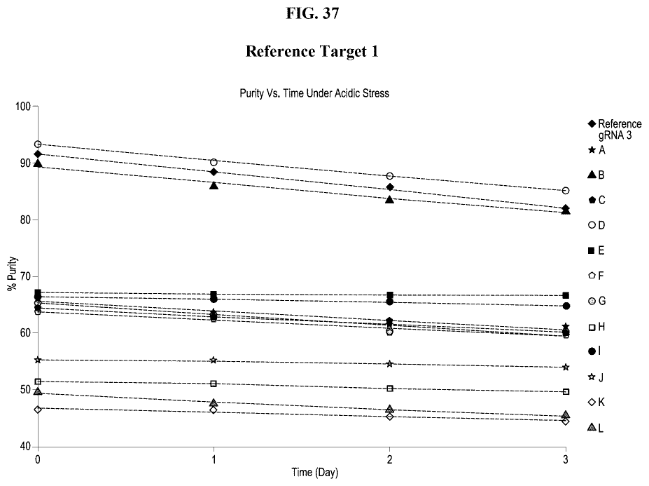

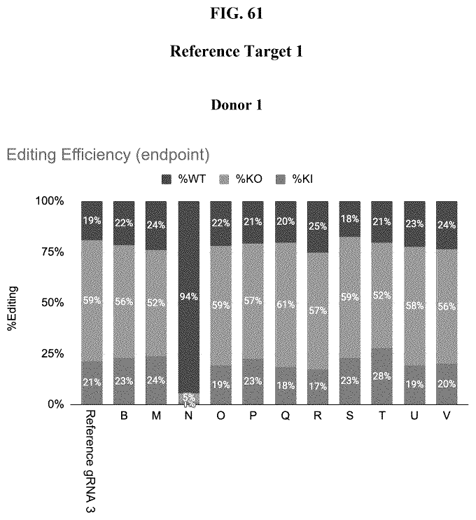

The present disclosure relates to modified guide RNA molecules, pharmaceutical compositions including such modified guide RNAs and methods for modifying a cell by administering the modified guide RNA molecules. The present disclosure is based, in part, on the discovery that the introduction of phosphorodithioate linkages into a gRNA molecule eliminates the chiral centers generated by phosphorothioate linkages resulting in stable gRNA molecules, gRNA compositions that do not include diastereomers at these modified phosphate positions, and better defined gRNA populations. As shown in FIGS. 37, 38, 57 and 58, gRNA molecules that include phosphorodithioate linkages at the 5′ and/or the 3′ termini result in increased stability under forced degradation conditions such as under basic stress conditions and acidic stress conditions compared to gRNA molecules that do not include phosphorodithioate linkages. Further, as shown in FIGS. 61, 65 and 69, gRNA molecules that include phosphorodithioate linkages at the 5′ and/or the 3′ termini have comparable or increased editing efficiency compared to gRNA molecules that do not include phosphorodithioate linkages.

For clarity, but not by way of limitation, the detailed description of the presently disclosed subject matter is divided into the following subsections:
I. Definitions;
II Modified Guide RNAs;
III. RNA-guided Nucleases;
IV. Compositions;
V. Methods of Use;
VI. Gene Editing Systems; and
VII. Exemplary Embodiments.

### I. DEFINITIONS

Unless defined otherwise, all technical and scientific terms used herein have the meaning commonly understood by a person skilled in the art to which the subject matter of the present disclosure belongs. The following references provide one of skill with a general definition of many of the terms used in the present disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the use of the word “a” or “an” when used in conjunction with the term “comprising” in the claims and/or the specification can mean “one,” but it is also consistent with the meaning of “one or more,” “at least one,” and “one or more than one.”

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “comprise(s),” “include(s),” “having,” “has,” “can,” “contain(s)” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “coupled” can refer to the connecting or uniting of two or more components by an interaction, bond, link, force or tie in order to keep two or more components together. In certain embodiments, the term “coupled” encompasses either direct or indirect binding where, for example, a first component is directly bound to a second component, or one or more intermediate molecules are disposed between the first component and the second component. Exemplary bonds comprise covalent bonds, ionic bonds, van der Waals interactions and other bonds identifiable by a skilled person.

The terms “detect” or “detection,” as used herein, indicate the determination of the existence and/or presence of a target, e.g., a nucleic acid target, in a limited portion of space, including but not limited to a sample. The terms “detect” or “detection,” as used herein, can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

As used herein, the term “domain” refers to a segment of a protein or nucleic acid, e.g., a gRNA molecule. Unless otherwise indicated, a domain is not required to have any specific functional property.

The term “editing efficiency,” as used herein, refers to the total number of sequence reads with insertions or deletions of nucleotides into a target region of interest over the total number of sequence reads following cleavage by an RNA-guided nuclease.

The terms “guide RNA,” “gRNA” or “gRNA molecule,” as used interchangeably herein, refer to a nucleic acid that promotes the specific targeting or homing of an RNA-guided nuclease to a target nucleic acid.

As used herein, the term “hybridization,” refers to the process in which two single-stranded polynucleotides bind non-covalently to form a stable double-stranded polynucleotide.

As used herein, the term “individual” or “subject” refers to a vertebrate or an invertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses, apes and monkeys. In certain embodiments, the individual or subject is a human.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, a “label” refers to an agent that allows for direct or indirect detection. Labels include, but are not limited to, fluorescent labels, chromogenic labels, electron dense labels, chemiluminescent labels and radioactive labels. Non-limiting examples of labels include green fluorescent protein (“GFP”), mCherry, dtTomato, or other fluorescent proteins known in the art (e.g., Shaner et al., A Guide to Choosing Fluorescent Proteins, Nature Methods 2(12):905-909 (2005) incorporated by reference herein, ³²P, ¹⁴C, ¹²⁵I, ³H and ¹³¹I, fluorogens (such as Rare Earth Chelate or lucifer yellow and its derivatives), Rhodamine (rhodamine) and its derivatives, dansyl, umbelliferone, luciferase (such as firefly luciferase and bacterial fluorescence plain enzyme) (U.S. Pat. No. 4,737,456), fluorescein, 2,3-dihydros phthalazine diketone, as well as enzymes producing detectable signals, e.g., horseradish peroxidase (HRP), alkaline phosphorus sour enzyme, beta galactosidase, glucoamylase, lysozyme, carbohydrate oxidase (such as glucose oxidase, galactose oxidase and glucose-6-phosphate dehydrogenase (G6PD)) and heterocyclic oxidases (such as uricase and xanthine oxidase).

The term “nucleic acid” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. The term nucleic acid encompasses deoxyribonucleic acid (DNA) including, e.g., complementary DNA (cDNA) and genomic DNA, ribonucleic acid (RNA), e.g., messenger RNA (mRNA), synthetic forms of DNA or RNA, and mixed polymers comprising two or more of these molecules. The nucleic acid molecule can be linear or circular. In addition, the term nucleic acid includes both, sense and antisense strands, as well as single stranded and double stranded forms. Moreover, the herein described nucleic acid can contain naturally occurring or non-naturally occurring nucleotides. Examples of non-naturally occurring nucleotides include modified nucleotide bases with derivatized sugars or phosphate backbone linkages or chemically modified residues.

The term “nucleoside” refers to a compound that includes a purine or pyrimidine base (i.e., cytosine (C), guanine (G), adenine (A), thymine (T) or uracil (U)) and a sugar (i.e., deoxyribose or ribose).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “plurality” refers to a number larger than one. In certain embodiments, the term “plurality of guide RNAs” refers to a number of guide RNAs larger than one. For example, but not by way of limitation, a plurality of guide RNAs includes at least two guide RNAs. In certain embodiments, the term “plurality of nucleic acids” refers to a number of nucleic acids larger than one. For example, but not by way of limitation, a plurality of nucleic acids includes at least two nucleic acids.

The term “reference molecule” or “control molecule,” e.g., a reference or control gRNA molecule, as used herein, refers to a molecule to which a subject molecule, e.g., a subject gRNA molecule, is compared. For example, but not by way of limitation, the subject gRNA molecule includes one or more modifications, e.g., nucleotide modifications, compared to a reference molecule.

The term “specifically binds,” as used herein, refers to the preferential binding to a target molecule, e.g., a protein or nucleic acid, relative to other molecules, e.g., proteins or nucleic acids, in a sample.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The decrease can be at least a 10%, 20%, 30%, 40%, 50%, 60%, 70%, 80%, 90%, 95%, 98% or 99% decrease in severity of complications, signs or symptoms or in likelihood of progression to another grade. “Treatment” can also refer to inhibiting proliferation of a cancer or progression to a higher grade by at least 10%, 20%, 30%, 40%, 50%, 60%, 70%, 80%, 90%, 95%, 98% or 99%. In certain embodiments, gRNAs of the present disclosure are used to delay development of a disease or to slow the progression of a disease.

The term “therapeutic effect,” as used herein, refers to a local or systemic effect in a subject caused by a pharmacologically active substance.

The terms “therapeutically-effective amount” and “effective amount,” as used herein, refer to the amount of a composition of the present disclosure which is effective for producing some desired therapeutic effect in at least a sub-population of cells in a subject at a reasonable benefit/risk ratio applicable to any medical treatment.

The term “variation,” when referring to a variation at a given polypeptide residue position, refers to any modifications at those residues. For example, but not by way of limitation, the term “variation” can refer to substituting an amino acid at a given position with a different amino acid.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. MODIFIED GUIDE RNA MOLECULES

The present disclosure provides gRNAs that include one or more modifications. In certain embodiments, the one or more modifications increases the stability of the gRNA as compared to a gRNA that does not include the one or more modifications, e.g., a control or reference gRNA. For example, but not by way of limitation, the modifications improve the stability of the disclosed gRNA molecules by preventing degradation of the gRNA molecules by nucleases, e.g., endonucleases and/or exonucleases. Such enhanced stability can improve the therapeutic efficacy of the modified gRNA molecules. In addition, the modifications can provide better shelf-life stability and result in gRNA compositions that have greater quality because diastereomers would not be present in such compositions. The modifications can also improve editing efficiency and reduce possible off-target effects.

In certain embodiments, the modified gRNAs of the present disclosure exhibit an increase in stability of about 1% or more, e.g., about 2% or more, about 3% or more, about 4% or more, about 5% or more, about 6% or more, about 7% or more, about 8% or more, about 9% or more, about 10% or more, about 15% or more, about 20% or more, about 25% or more, about 30% or more, about 35% or more, about 40% or more, about 45% or more, about 50% or more or about 55% or more, compared to a reference gRNA under degradative conditions, e.g., basic stress conditions and/or acidic stress conditions. In certain embodiments, the modified gRNAs of the present disclosure exhibit an increase in stability from about 1% to about 60% compared to a reference gRNA under degradative conditions, e.g., basic stress conditions and/or acidic stress conditions. In certain embodiments, the modified gRNAs of the present disclosure exhibit an increase in stability of about 1% or more, about 2% or more, about 3% or more, about 4% or more or about 5% or more under acidic stress conditions. In certain embodiments, the modified gRNAs of the present disclosure exhibit an increase in stability from about 1% to about 5% compared to a reference gRNA under acidic stress conditions. In certain embodiments, the modified gRNAs of the present disclosure exhibit an increase in stability of about 5% or more, about 10% or more, about 15% or more, about 20% or more, about 25% or more, about 30% or more, about 35% or more, about 40% or more, about 45% or more, about 50% or more or about 55% or more under basic stress conditions. In certain embodiments, the modified gRNAs of the present disclosure exhibit an increase in stability from about 1% to about 60% compared to a reference gRNA under basic stress conditions. In certain embodiments, the modified gRNAs of the present disclosure exhibit a reduction in degradation of about 1% or more, e.g., about 2% or more, about 3% or more, about 4% or more, about 5% or more, about 6% or more, about 7% or more, about 8% or more, about 9% or more, about 10% or more, about 15% or more, about 20% or more, about 25% or more, about 30% or more, about 35% or more, about 40% or more, about 45% or more, about 50% or more or about 55% or more, compared to a reference gRNA under degradative conditions, e.g., basic stress conditions and/or acidic stress conditions. In certain embodiments, the modified gRNAs of the present disclosure exhibit a reduction in degradation from about 1% to about 60% compared to a reference gRNA under degradative conditions, e.g., basic stress conditions and/or acidic stress conditions. In certain embodiments, the modified gRNAs of the present disclosure exhibit a reduction in degradation of about 1% or more, about 2% or more, about 3% or more, about 4% or more or about 5% or more under acidic stress conditions. In certain embodiments, the modified gRNAs of the present disclosure exhibit a reduction in degradation from about 1% to about 5% compared to a reference gRNA under acidic stress conditions. In certain embodiments, the modified gRNAs of the present disclosure exhibit a reduction in degradation of about 5% or more, about 10% or more, about 15% or more, about 20% or more, about 25% or more, about 30% or more, about 35% or more, about 40% or more, about 45% or more, about 50% or more or about 55% or more under basic stress conditions. In certain embodiments, the modified gRNAs of the present disclosure exhibit a reduction in degradation from about 1% to about 60% compared to a reference gRNA under basic stress conditions. In certain embodiments, the increase in stability and/or reduction in degradation is observed about 24 hours, 48 hours, 3 days, 4 days, 5 days, 6 days or 7 days after being subjected to the degradation conditions. In certain embodiments, thermal stress conditions include subjecting the gRNA to high temperatures, e.g., temperatures higher than about 37° C. In certain embodiments, oxidative stress conditions include subjecting the gRNA to free radicals and/or compounds (e.g., H₂O₂). In certain embodiments, basic stress conditions include subjecting the gRNA to a basic pH, e.g., a pH greater than about 8, e.g., about 11. In certain embodiments, acidic stress conditions include subjecting the gRNA to an acidic pH, e.g., a pH less than about 6, e.g., about 5. In certain embodiments, the reference gRNA can be a gRNA that has the same nucleotide sequence as the modified gRNA (e.g., without the same modifications as the modified gRNA). In certain embodiments, the reference gRNA can be a gRNA that has a targeting domain with the same sequence as the modified gRNA (e.g., without the same modifications as the modified gRNA). In certain embodiments, the reference gRNA can be a gRNA that has the same number of nucleotides as the modified gRNA (e.g., without the same modifications as the modified gRNA).

In certain embodiments, the gRNAs of the present disclosure include one or more, two or more, three or more, four or more, five or more, six or more, seven or more, eight or more, nine or more, ten or more, eleven or more, twelve or more, thirteen or more, fourteen or more or fifteen or more modifications. In certain embodiments, the gRNAs of the present disclosure include at least two, at least three, at least four, at least five, at least six, at least seven, at least eight, at least nine, at least ten nucleotide modifications, at least eleven nucleotide modifications, at least twelve nucleotide modifications, at least thirteen nucleotide modifications, at least fourteen nucleotide modifications or at least fifteen nucleotide modifications. In certain embodiments, the gRNAs of the present disclosure include at least about eleven modifications.

In certain embodiments, a gRNA of the present disclosure has a length from about 20 to about 200 nucleotides, e.g., from about 20 to about 190, from about 20 to about 180, from about 20 to about 170, from about 20 to about 160, from about 20 to about 150, from about 20 to about 140, from about 20 to about 130, from about 20 to about 120, from about 20 to about 110, from about 20 to about 100, from about 30 to about 200, from about 40 to about 200, from about 50 to about 200, from about 60 to about 200, from about 70 to about 200, from about 80 to about 200, from about 90 to about 200, from about 50 to about 150, from about 80 to about 120 or from about 90 to about 100 nucleotides. In certain embodiments, a gRNA of the present disclosure has a length from about 80 to about 120 nucleotides. In certain embodiments, a gRNA of the present disclosure is about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200 or more nucleotides in length.

In certain embodiments, about 1% to about 20% of the nucleotides present in the gRNAs of the present disclosure are modified, e.g., from about 1% to about 19%, about 1% to about 18%, about 1% to about 17%, about 1% to about 16%, about 1% to about 15%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 20%, about 3% to about 20%, about 4% to about 20%, about 5% to about 20%, about 6% to about 20%, about 7% to about 20%, about 8% to about 20%, about 9% to about 20%, about 10% to about 20%, about 11% to about 20%, about 12% to about 20%, about 13% to about 20%, about 14% to about 20%, about 15% to about 20%, about 16% to about 20%, about 17% to about 20%, about 18% to about 20%, about 19% to about 20%, about 5% to about 15% or about 10% to about 15% of the nucleotides present in the gRNAs of the present disclosure are modified. In certain embodiments, about 1% to about 15% of the nucleotides present in the gRNAs of the present disclosure are modified. In certain embodiments, about 1% to about 10% of the nucleotides present in the gRNAs of the present disclosure are modified. In certain embodiments, about 1% to about 6% of the nucleotides present in the gRNAs of the present disclosure are modified. In certain embodiments, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19% or about 20% or more of the nucleotides present in the gRNAs are modified.

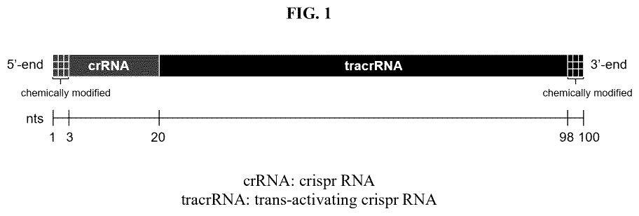

In certain embodiments, gRNA molecules of the present disclosure have the structure shown in FIG. 1. For example, but not by way of limitation, a gRNA molecule of the present disclosure includes a spacer region (blue region in FIG. 1) at its 5′ terminus. An additional exemplary structure of a gRNA molecule of the present disclosure is provided in FIG. 1A of Daniel et al., Frontiers in Genome Editing 2:617910 (2021), the contents of which are incorporated herein by reference in their entirety (the spacer region is noted in blue and red in FIG. 1A in Daniel et al.).

In certain embodiments, a gRNA molecule of the present disclosure includes one or more modifications in the spacer region of the gRNA molecule. In certain embodiments, the spacer region of a presently disclosed gRNA molecule is complementary, e.g., at least about 80%, about 85%, about 90%, about 95%, about 98%, about 99% or about 100% complementary, to a sequence of a target nucleic acid. In certain embodiments, the spacer region includes the first 30 nucleotides, e.g., the first 29 nucleotides, the first 28 nucleotides, the first 27 nucleotides, the first 26 nucleotides, the first 25 nucleotides, the first 24 nucleotides, the first 23 nucleotides, the first 22 nucleotides, the first 21 nucleotides, the first 20 nucleotides, the first 19 nucleotides, the first 18 nucleotides, the first 17 nucleotides, the first 16 nucleotides or the first 15 nucleotides, present at the 5′ terminus of a gRNA molecule. In certain embodiments, the spacer region includes the first 20 nucleotides present at the 5′ terminus of a gRNA molecule. In certain embodiments, the modifications in the spacer region do not interfere with the editing efficiency, which can be evaluated using techniques know in the art or described herein. In certain embodiments, the modifications in the spacer region can improve the editing efficiency and reduce off-target effects. In certain embodiments, the spacer region includes 1, 2, 3, 4, 5, 6, 7, 8, 9 or 10 modifications. In certain embodiments, the spacer region includes 1, 2, 3, 4, 5, 6, 7, 8, 9 or 10 modifications within the first 5 nucleotides at its 5′ terminus. In certain embodiments, the spacer region includes 1, 2, 3, 4, 5 or 6 modifications within the first 5 nucleotides at its 5′ terminus. In certain embodiments, the spacer region includes 1, 2, 3, 4, 5, 6, 7, 8, 9 or 10 modifications within the first 3 nucleotides at its 5′ terminus. In certain embodiments, the spacer region includes 1, 2, 3, 4, 5 or 6 modifications within the first 3 nucleotides at its 5′ terminus. In certain embodiments, the spacer region includes at least one chemically modified nucleotide within the first 5 nucleotides at its 5′ terminus. In certain embodiments, the spacer region includes 1, 2, 3, 4 or 5 chemically modified nucleotides within the first 5 nucleotides at its 5′ terminus. In certain embodiments, the spacer region includes 1 chemically modified nucleotide within the first 5 nucleotides at its 5′ terminus. In certain embodiments, the spacer region includes 2 chemically modified nucleotides within the first 5 nucleotides at its 5′ terminus. In certain embodiments, the spacer region includes 3 chemically modified nucleotides within the first 5 nucleotides at its 5′ terminus. In certain embodiments, the spacer region includes 4 chemically modified nucleotides within the first 5 nucleotides at its 5′ terminus. In certain embodiments, the spacer region includes 5 chemically modified nucleotides within the first 5 nucleotides at its 5′ terminus. In certain embodiments, the spacer region includes at least one chemically modified nucleotide within the first 3 nucleotides at its 5′ terminus. In certain embodiments, the spacer region includes 1, 2 or 3 chemically modified nucleotides within the first 3 nucleotides at its 5′ terminus. In certain embodiments, the spacer region includes 1 chemically modified nucleotide within the first 3 nucleotides at its 5′ terminus. In certain embodiments, the spacer region includes 2 chemically modified nucleotides within the first 3 nucleotides at its 5′ terminus. In certain embodiments, the spacer region includes 3 chemically modified nucleotides within the first 3 nucleotides at its 5′ terminus. In certain embodiments, a chemically modified nucleotide can include one or more modifications, two or more modifications or three or more modifications. In certain embodiments, a chemically modified nucleotide can include one modification, e.g., a sugar modification or a phosphate backbone modification as described below. In certain embodiments, a chemically modified nucleotide can include two modifications, e.g., a sugar modification and a phosphate backbone modification as described below. In certain embodiments, a chemically modified nucleotide can include three modifications, e.g., two sugar modifications and a phosphate backbone modification as described below.

In certain embodiments, a gRNA molecule of the present disclosure includes one or more modifications at its 5′ terminus. In certain embodiments, the term “5′ terminus” as used herein with respect to a modification refers to the first 5 nucleotides at the 5′ terminus of a gRNA molecule. For example, but not by way of limitation, one or more nucleotides present at the 5′ terminus of a gRNA molecule of the present disclosure is modified. In certain embodiments, the first nucleotide present at the 5′ terminus of a gRNA molecule of the present disclosure is modified. In certain embodiments, the second nucleotide present at the 5′ terminus of a gRNA molecule of the present disclosure is modified. In certain embodiments, the third nucleotide present at the 5′ terminus of a gRNA molecule of the present disclosure is modified. In certain embodiments, the fourth nucleotide present at the 5′ terminus of a gRNA molecule of the present disclosure is modified. In certain embodiments, the fifth nucleotide present at the 5′ terminus of a gRNA molecule of the present disclosure is modified. In certain embodiments, a gRNA molecule of the present disclosure includes 1, 2, 3, 4, 5, 6, 7, 8, 9 or 10 modifications within the first 5 nucleotides at its 5′ terminus.

In certain embodiments, a gRNA molecule of the present disclosure includes modifications at two consecutive nucleotides, e.g., two consecutive nucleotides that are within the first 5 nucleotides of the 5′ terminus of the gRNA molecule. For example, but not by way of limitation, the first and second nucleotides of the gRNA molecule are modified. In certain embodiments, the first and third nucleotides of the gRNA molecule are modified. In certain embodiments, the second and third nucleotides at the 5′ terminus of the gRNA molecule are modified. In certain embodiments, the third and fourth nucleotides at the 5′ terminus of the gRNA molecule are modified. In certain embodiments, the fourth and fifth nucleotides at the 5′ terminus of the gRNA molecule are modified.

In certain embodiments, a gRNA molecule of the present disclosure includes modifications at three consecutive nucleotides, e.g., three consecutive nucleotides that are within the first 5 nucleotides of the 5′ terminus of the gRNA molecule. For example, but not by way of limitation, the first, second and third nucleotides at the 5′ terminus of the gRNA molecule are modified. In certain embodiments, the second, third and fourth nucleotides at the 5′ terminus of the gRNA molecule are modified. In certain embodiments, the third, fourth and fifth nucleotides at the 5′ terminus of the gRNA molecule are modified.

In certain embodiments, a gRNA molecule of the present disclosure includes modifications at four consecutive nucleotides, e.g., four consecutive nucleotides that are within the first 5 nucleotides of the 5′ terminus of the gRNA molecule. For example, but not by way of limitation, the first, second, third and fourth nucleotides at the 5′ terminus of the gRNA molecule are modified. In certain embodiments, the second, third, fourth and fifth nucleotides at the 5′ terminus of the gRNA molecule are modified.

In certain embodiments, a gRNA molecule of the present disclosure includes modifications at five consecutive nucleotides, e.g., five consecutive nucleotides that are within the first 5 nucleotides of the 5′ terminus of the gRNA molecule. For example, but not by way of limitation, the first, second, third, fourth and fifth nucleotides at the 5′ terminus of the gRNA molecule are modified.

In certain embodiments, a gRNA molecule of the present disclosure includes one or more modifications at its 3′ terminus. In certain embodiments, the term “3′ terminus” as used herein with respect to a modification refers to the last 5 nucleotides (n, n−1, n−2, n−3 and n−4 nucleotides) at the 3′ terminus of a gRNA molecule. For example, but not by way of limitation, one or more nucleotides present at the 3′ terminus of a gRNA molecule of the present disclosure is modified. In certain embodiments, the last nucleotide (n) present at the 3′ terminus of a gRNA molecule of the present disclosure is modified. In certain embodiments, the second to last nucleotide (n−1) present at the 3′ terminus of a gRNA molecule of the present disclosure is modified. In certain embodiments, the third to last nucleotide (n−2) present at the 3′ terminus of a gRNA molecule of the present disclosure is modified. In certain embodiments, the fourth to last nucleotide (n−3) present at the 3′ terminus of a gRNA molecule of the present disclosure is modified. In certain embodiments, the fifth to last nucleotide (n−4) present at the 3′ terminus of a gRNA molecule of the present disclosure is modified. In certain embodiments, a gRNA molecule of the present disclosure includes 1, 2, 3, 4, 5, 6, 7, 8, 9 or 10 modifications within the last 5 nucleotides at its 3′ terminus.

In certain embodiments, a gRNA molecule of the present disclosure includes modifications at two consecutive nucleotides, e.g., two consecutive nucleotides that are within 5 nucleotides of the 3′ terminus of the gRNA molecule. For example, but not by way of limitation, the n and n−1 nucleotides of the 3′ terminus of the gRNA molecule are modified. In certain embodiments, the n and n−2 nucleotides of the 3′ terminus of the gRNA molecule are modified. In certain embodiments, the n−1 and n−2 nucleotides of the 3′ terminus of the gRNA molecule are modified. In certain embodiments, the n−2 and n−3 nucleotides of the 3′ terminus of the gRNA molecule are modified. In certain embodiments, the n−3 and n−4 nucleotides of the 3′ terminus of the gRNA molecule are modified. In certain embodiments, the n−4 and n−5 nucleotides of the 3′ terminus of the gRNA molecule are modified.

In certain embodiments, a gRNA molecule of the present disclosure includes modifications at three consecutive nucleotides, e.g., three consecutive nucleotides that are within the last 5 nucleotides of the 3′ terminus of the gRNA molecule. For example, but not by way of limitation, the n, n−1 and n−2 nucleotides of the gRNA molecule are modified. In certain embodiments, the n−1, n−2 and n−3 nucleotides of the 3′ terminus of the gRNA molecule are modified. In certain embodiments, the n−2, n−3 and n−4 nucleotides of the 3′ terminus of the gRNA molecule are modified.

In certain embodiments, a gRNA molecule of the present disclosure includes modifications at four consecutive nucleotides, e.g., four consecutive nucleotides that are within 5 nucleotides of the 3′ terminus of the gRNA molecule. For example, but not by way of limitation, the n, n−1, n−2 and n−3 nucleotides of the gRNA molecule are modified. In certain embodiments, the n−1, n−2, n−3 and n−4 nucleotides of the gRNA molecule are modified.

In certain embodiments, a gRNA molecule of the present disclosure includes modifications at five consecutive nucleotides, e.g., five consecutive nucleotides that are within 5 nucleotides of the 3′ terminus of the gRNA molecule. For example, but not by way of limitation, the n, n−1, n−2, n−3 and n−4 nucleotides of the gRNA molecule are modified.

In certain embodiments, a gRNA molecule of the present disclosure includes one or more modifications at its 5′ terminus and one or more modifications at its 3′ terminus. In certain embodiments, a gRNA molecule of the present disclosure includes two or more modifications at its 5′ terminus and two or more modifications at its 3′ terminus. In certain embodiments, a gRNA molecule of the present disclosure includes three or more modifications at its 5′ terminus and three or more modifications at its 3′ terminus. In certain embodiments, a gRNA molecule of the present disclosure includes four or more modifications at its 5′ terminus and four or more modifications at its 3′ terminus. In certain embodiments, a gRNA molecule of the present disclosure includes five or more modifications at its 5′ terminus and five or more modifications at its 3′ terminus.

Non-limiting examples of modifications that can be included in a gRNA molecule of the present disclosure are disclosed herein. In certain embodiments, a gRNA of the present disclosure can include one or more of the following modifications: (i) a phosphate backbone modification and (ii) a sugar modification.

### Phosphate Backbone Modifications

In certain embodiments, the phosphate group of a nucleotide present within a gRNA molecule can be modified. For example, but not by way of limitation, the phosphate group of a nucleotide can be modified by replacing one or more of the oxygens, e.g., bridging or non-bringing oxygens, in a phosphodiester linkage with a different substituent. Non-limiting examples of substituents include sulfur (S), nitrogen (N), hydrogen (H) and carbon (C). In certain embodiments, one or more oxygens in a phosphodiester linkage are substituted with S.

In certain embodiments, a gRNA molecule can be modified with one or more phosphorothioate (PS) linkages. In certain embodiments, a PS linkage or bond refers to a bond where a sulfur is substituted for one non-bridging phosphate oxygen in an internucleotide phosphodiester linkage. In certain embodiments, “*” is used herein to denote a nucleotide that is linked to the adjacent 3′ nucleotide with a PS linkage. In certain embodiments, the phosphorous of an unmodified phosphodiester linkage is achiral and the replacement of one non-bridging phosphate oxygen with sulfur renders the phosphorous chiral.

In certain embodiments, a gRNA molecule can be modified with one or more phosphorodithioate (PS2) linkages. In certain embodiments, a PS2 linkage or bond refers to a bond where both non-bridging oxygens in an internucleotide phosphodiester linkage are replaced by sulfur. In certain embodiments, “**” is used herein to denote a nucleotide that is linked to the adjacent 3′ nucleotide with a PS2 linkage. Similar to a naturally-occurring phosphodiester backbone linkage, a PS2 linkage is achiral at the phosphorus resulting in gRNA molecules that are not diastereomer at the PS2 linkage. In certain embodiments, phosphorodithioate linkages are resistant to nuclease degradation and the presence of one or more phosphorodithioate linkages in a gRNA of the present disclosure can increase the stability of the gRNA, e.g., compared to a non-modified gRNA.

### Sugar Modifications

In certain embodiments, the sugar group of a nucleotide present in a gRNA of the present disclosure can be modified. For example, but not by way of limitation, a nucleotide of a gRNA of the present disclosure can include one or more modifications to its sugar group, e.g., ribose.

In certain embodiments, a sugar group can be modified at the 2′ hydroxyl group (OH). In certain embodiments, the 2′ hydroxyl group can be replaced with a different substituent. Non-limiting examples of substituents include hydrogen (H), a halogen, an alkyl or an alkoxy (OR, where R can be an alkyl, a cycloalkyl or an alkoxy).

In certain embodiments, the 2′ hydroxyl group is substituted with an alkoxy group. In certain embodiments, the 2′ hydroxyl group is substituted with a methoxy group. In certain embodiments, “m” is used herein to denote a nucleotide that is modified with a 2′-O-methyl (i.e., a 2′-O-methyl modified nucleotide).

In certain embodiments, the hydrogen (H) of the 2′ hydroxyl group is substituted with a methoxyethyl group. In certain embodiments, “M” is used herein to denote a nucleotide that is modified with a 2′-O-(2-methoxyethyl) (i.e., 2′-O-(2-methoxyethyl) modified nucleotide).

In certain embodiments, the 2′ hydroxyl group can be substituted with a halogen. Non-limiting examples of halogens include fluorine (F), chlorine (Cl), bromide (Br) and iodine (I). In certain embodiments, the 2′ hydroxyl group is replaced with a fluorine. In certain embodiments, “f” is used herein to denote a nucleotide that is modified with a 2′-fluoro (i.e., a 2′-fluoro modified nucleotide).

In certain embodiments, the 2′ hydroxyl group can be replaced with a hydrogen (H) to generate a deoxyribose sugar. For example, but not by way of limitation, a nucleotide present in a gRNA of the present disclosure can have a deoxyribose sugar. In certain embodiments, “d” is used herein to denote a nucleotide that has a deoxyribose sugar.

In certain embodiments, modification of the 2′ hydroxyl group can include “locked nucleic acids” (LNA) in which the 2′ hydroxyl group is connected to the 4′ carbon of the same ribose sugar. In certain embodiments, the 2′ hydroxyl group is connected to the 4′ carbon by a bridge, e.g., an alkylene (e.g., methylene), ether or amino bridge. In certain embodiments, “LNA” is used herein to denote a nucleotide that is an LNA.

Exemplary Modified gRNAs

In certain embodiments, a gRNA of the present disclosure can have at least one phosphate backbone modification, e.g., a phosphorodithioate linkage. In certain embodiments, a gRNA can have at least two phosphate backbone modifications, e.g., phosphorodithioate linkages. In certain embodiments, a gRNA can have at least three phosphate backbone modifications, e.g., phosphorodithioate linkages. In certain embodiments, a gRNA can have at least four phosphate backbone modifications, e.g., phosphorodithioate linkages. In certain embodiments, a gRNA can have at least five phosphate backbone modifications, e.g., phosphorodithioate linkages. In certain embodiments, a gRNA can have at least six phosphate backbone modifications, e.g., phosphorodithioate linkages. In certain embodiments, a gRNA can have at least seven phosphate backbone modifications, e.g., phosphorodithioate linkages. In certain embodiments, a gRNA can have at least eight phosphate backbone modifications, e.g., phosphorodithioate linkages. In certain embodiments, a gRNA can have at least nine phosphate backbone modifications, e.g., phosphorodithioate linkages. In certain embodiments, a gRNA can have at least ten phosphate backbone modifications, e.g., phosphorodithioate linkages. In certain embodiments, a gRNA can have from about 1 to about 10 phosphate backbone modifications, e.g., from about 1 to about 9, from about 1 to about 8, from about 1 to about 7, from about 1 to about 6, from about 1 to about 5, from about 1 to about 4, from about 1 to about 3, from about 1 to about 2, from about 2 to about 10, from about 3 to about 10, from about 4 to about 10, from about 5 to about 10, from about 6 to about 10, from about 7 to about 10, from about 8 to about 10, from about 9 to about 10, from about 2 to about 8, from about 2 to about 6, from about 3 to about 8, from about 3 to about 6 or from 3 to 5 phosphate backbone modifications, e.g., phosphorodithioate linkages. In certain embodiments, a gRNA can have from about 2 to about 10 phosphate backbone modifications, e.g., phosphorodithioate linkages. In certain embodiments, a gRNA can have from about 2 to about 9 phosphate backbone modifications, e.g., phosphorodithioate linkages. In certain embodiments, a gRNA can have from about 3 to about 8 phosphate backbone modifications, e.g., phosphorodithioate linkages. In certain embodiments, a gRNA can have from about 3 to about 5 phosphate backbone modifications, e.g., phosphorodithioate linkages.

In certain embodiments, a gRNA of the present disclosure can include at least one phosphorodithioate linkage and at least one phosphorothioate linkage, e.g., as shown in Table 10. For example, but not by way of limitation, a gRNA of the present disclosure can include at least two phosphorodithioate linkages and at least one phosphorothioate linkage. In certain embodiments, a gRNA of the present disclosure can include at least three phosphorodithioate linkages and at least one phosphorothioate linkage. In certain embodiments, a gRNA of the present disclosure can include at least four phosphorodithioate linkages and at least one phosphorothioate linkage. In certain embodiments, a gRNA of the present disclosure can include at least two phosphorodithioate linkages and at least two phosphorothioate linkages. In certain embodiments, a gRNA of the present disclosure can include at least two phosphorodithioate linkages and at least three phosphorothioate linkages.

In certain embodiments, a gRNA can have at least one sugar modification. In certain embodiments, a gRNA can have at least two sugar modifications. In certain embodiments, a gRNA can have at least three sugar modifications. In certain embodiments, a gRNA can have at least four sugar modifications. In certain embodiments, a gRNA can have at least five sugar modifications. In certain embodiments, a gRNA can have at least six sugar modifications. In certain embodiments, a gRNA can have at least seven sugar modifications. In certain embodiments, a gRNA can have at least eight sugar modifications. In certain embodiments, a gRNA can have at least nine sugar modifications. In certain embodiments, a gRNA can have at least ten sugar modifications. In certain embodiments, a gRNA can have from about 1 to about 10 sugar modifications, e.g., from about 1 to about 9, from about 1 to about 8, from about 1 to about 7, from about 1 to about 6, from about 1 to about 5, from about 1 to about 4, from about 1 to about 3, from about 1 to about 2, from about 2 to about 10, from about 3 to about 10, from about 4 to about 10, from about 5 to about 10, from about 6 to about 10, from about 7 to about 10, from about 8 to about 10, from about 9 to about 10, from about 2 to about 8, from about 2 to about 6, from about 3 to about 8 or from about 3 to about 6 sugar modifications. In certain embodiments, a gRNA can have from about 2 to about 10 sugar modifications. In certain embodiments, a gRNA can have from about 3 to about 8 sugar modifications. In certain embodiments, a gRNA molecule of the present disclosure can have from about 3 to about 6 sugar modifications.

In certain embodiments, a gRNA can have at least one phosphate backbone modification (e.g., at least two, at least three, at least four, at least five, at least six, at least seven, at least eight, at least nine or at least ten phosphate backbone modifications) and at least one sugar modification (e.g., at least two, at least three, at least four, at least five, at least six, at least seven, at least eight, at least nine or at least ten sugar modifications).

In certain embodiments, a gRNA can have at least two phosphate backbone modifications and at least two sugar modifications. In certain embodiments, a gRNA can have at least three phosphate backbone modifications and at least three sugar modifications. In certain embodiments, a gRNA can have at least four phosphate backbone modifications and at least four sugar modifications. In certain embodiments, a gRNA can have at least five phosphate backbone modifications and at least five sugar modifications. In certain embodiments, a gRNA can have at least five phosphate backbone modifications and at least six sugar modifications. In certain embodiments, a gRNA can have at least six phosphate backbone modifications and at least six sugar modifications. In certain embodiments, a gRNA can have at least seven phosphate backbone modifications and at least seven sugar modifications. In certain embodiments, a gRNA can have at least eight phosphate backbone modifications and at least eight sugar modifications. In certain embodiments, a gRNA can have at least eight phosphate backbone modifications and at least nine sugar modifications. In certain embodiments, a gRNA can have at least nine phosphate backbone modifications and at least nine sugar modifications. In certain embodiments, a gRNA can have at least ten phosphate backbone modifications and at least ten sugar modifications.

In certain embodiments, a gRNA molecule of the present disclosure includes at least one phosphorodithioate linkage at the 5′ terminus of the gRNA molecule. In certain embodiments, a gRNA molecule of the present disclosure includes a phosphorodithioate linkage between the first and second nucleotides at the 5′ terminus of the gRNA molecule. In certain embodiments, a gRNA molecule of the present disclosure includes a phosphorodithioate linkage between the second and third nucleotides at the 5′ terminus of the gRNA molecule. In certain embodiments, a gRNA molecule of the present disclosure includes a phosphorodithioate linkage between the third and fourth nucleotides at the 5′ terminus of the gRNA molecule. In certain embodiments, a gRNA molecule of the present disclosure includes a phosphorodithioate linkage between the fourth and fifth nucleotides at the 5′ terminus of the gRNA molecule.

In certain embodiments, a gRNA molecule of the present disclosure includes at least one phosphorodithioate linkage at the 3′ terminus of the gRNA molecule. In certain embodiments, a gRNA molecule of the present disclosure includes a phosphorodithioate linkage between the 3′ terminal (“n”) nucleotide and the n−1 nucleotide of the gRNA molecule. In certain embodiments, a gRNA molecule of the present disclosure includes a phosphorodithioate linkage between the n−1 and n−2 nucleotides at the 3′ terminus of the gRNA molecule. In certain embodiments, a gRNA molecule of the present disclosure includes a phosphorodithioate linkage between the n−2 and n−3 nucleotides at the 3′ terminus of the gRNA molecule. In certain embodiments, a gRNA molecule of the present disclosure includes a phosphorodithioate linkage between the n−3 and n−4 nucleotides at the 3′ terminus of the gRNA molecule.

In certain embodiments, a gRNA molecule of the present disclosure includes a phosphorodithioate linkage between the first and second nucleotides at the 5′ terminus of the gRNA molecule and a phosphorodithioate linkage between the n and n−1 nucleotides of the gRNA molecule.

In certain embodiments, a gRNA molecule of the present disclosure includes phosphorodithioate linkages between the first and second nucleotides and the second and third nucleotides at the 5′ terminus of the gRNA molecule.

In certain embodiments, a gRNA molecule of the present disclosure includes phosphorodithioate linkages between the first and second nucleotides and the third and fourth nucleotides at the 5′ terminus of the gRNA molecule.

In certain embodiments, a gRNA molecule of the present disclosure includes phosphorodithioate linkages between the second and third nucleotides and the third and fourth nucleotides at the 5′ terminus of the gRNA molecule.

In certain embodiments, a gRNA molecule of the present disclosure includes phosphorodithioate linkages between the first and second nucleotides, the second and third nucleotides and the third and fourth nucleotides at the 5′ terminus of the gRNA molecule.

In certain embodiments, a gRNA molecule of the present disclosure includes phosphorodithioate linkages between the first and second nucleotides, the second and third nucleotides, the third and fourth nucleotides and the fourth and fifth nucleotides at the 5′ terminus of the gRNA molecule.

In certain embodiments, a gRNA molecule of the present disclosure includes a phosphorothioate linkage between the first and second nucleotides.

In certain embodiments, a gRNA molecule of the present disclosure includes a phosphorothioate linkage between the third and fourth nucleotides.

In certain embodiments, a gRNA molecule of the present disclosure includes a phosphorothioate linkage between the first and second nucleotides, the second and third nucleotides and the third and fourth nucleotides.

In certain embodiments, a gRNA molecule of the present disclosure includes phosphorodithioate linkages between the n and n−1 nucleotides and the n−1 and n−2 nucleotides.

In certain embodiments, a gRNA molecule of the present disclosure includes phosphorodithioate linkages between the n and n−1 nucleotides and the n−2 and n−3 nucleotides of the gRNA molecule.

In certain embodiments, a gRNA molecule of the present disclosure includes phosphorodithioate linkages between the n−1 and n−2 nucleotides and the n−2 and n−3 nucleotides of the gRNA molecule.

In certain embodiments, a gRNA molecule of the present disclosure includes phosphorodithioate linkages between the n and n−1, n−1 and n−2 nucleotides, the n−2 and n−3 and the n−3 and n−4 nucleotides of the gRNA molecule.

In certain embodiments, a gRNA molecule of the present disclosure includes phosphorodithioate linkages between the n−1 and n−2 nucleotides, the n−2 and n−3, the n−3 and n−4 and the n−4 and n−5 nucleotides of the gRNA molecule.

In certain embodiments, a gRNA molecule of the present disclosure includes a phosphorothioate linkage between the n and n−1 nucleotides.

In certain embodiments, a gRNA molecule of the present disclosure includes a phosphorothioate linkage between the n−1 and n−2 nucleotides.

In certain embodiments, a gRNA molecule of the present disclosure includes a phosphorothioate linkage between the n−1 and n−2 nucleotides and a phosphorodithioate linkage between the n and n−1 nucleotides. In certain embodiments, the gRNA further includes phosphorothioate linkages between the first and second nucleotides, the second and third nucleotides and the third and fourth nucleotides.

In certain embodiments, a gRNA molecule of the present disclosure includes a phosphorothioate linkage between the third and fourth nucleotides and phosphorodithioate linkages between the first and second nucleotides and the second and third nucleotides. In certain embodiments, the gRNA further includes phosphorodithioate linkages between n and n−1 and between n−1 and n−2 nucleotides.

In certain embodiments, a gRNA molecule of the present disclosure includes phosphorothioate linkages between the first and second nucleotides, the second and third nucleotides and the third and fourth nucleotides. In certain embodiments, the gRNA further includes phosphorodithioate linkages between n and n−1 and between n−1 and n−2 nucleotides.

In certain embodiments, a gRNA molecule of the present disclosure includes one or more of a 2′-fluoro modified nucleotide, a 2′-O-methyl modified nucleotide, a 2′-O-(2-methoxyethyl) modified nucleotide, a deoxyribose nucleotide and a locked nucleic acid (LNA) at its 5′ terminus, e.g., within the first 5 nucleotides at the 5′ terminus. For example, but not by way of limitation, the first nucleotide at the 5′ terminus of the gRNA molecule is a 2′-fluoro modified nucleotide, a 2′-O-methyl modified nucleotide, a 2′-O-(2-methoxyethyl) modified nucleotide, a deoxyribose nucleotide or a locked nucleic acid (LNA). In certain embodiments, the first nucleotide at the 5′ terminus of the gRNA molecule is a 2′-fluoro modified nucleotide. In certain embodiments, the first nucleotide at the 5′ terminus of the guide RNA molecule is a 2′-O-methyl modified nucleotide. In certain embodiments, the first nucleotide at the 5′ terminus of the gRNA molecule is a 2′-O-(2-methoxyethyl) modified nucleotide. In certain embodiments, the first nucleotide at the 5′ terminus of the gRNA molecule is an LNA. In certain embodiments, the first nucleotide at the 5′ terminus of the gRNA molecule has a deoxyribose sugar.

In certain embodiments, a gRNA of the present disclosure includes two or more 2′-fluoro modified nucleotides, 2′-O-methyl modified nucleotides, 2′-O-(2-methoxyethyl) modified nucleotides, deoxyribose nucleotides and/or LNAs at its 5′ terminus, e.g., within the first 5 nucleotides at the 5′ terminus. For example, but not by way of limitation, the first and second nucleotides at the 5′ terminus of the gRNA molecule are 2′-fluoro modified nucleotides, 2′-O-methyl modified nucleotides, 2′-O-(2-methoxyethyl) modified nucleotides, deoxyribose nucleotides, LNAs or a combination thereof. In certain embodiments, the first and second nucleotides at the 5′ terminus of the gRNA molecule are 2′-fluoro modified nucleotides. In certain embodiments, the first and second nucleotides at the 5′ terminus of the guide RNA molecule are 2′-O-methyl modified nucleotides. In certain embodiments, the first and second nucleotides at the terminus of the gRNA molecule are 2′-O-(2-methoxyethyl) modified nucleotides. In certain embodiments, the first and second nucleotides at the 5′ terminus of the gRNA molecule are LNAs. In certain embodiments, the first and second nucleotides at the 5′ terminus of the gRNA molecule have a deoxyribose sugar.

In certain embodiments, a gRNA of the present disclosure includes three or more 2′-fluoro modified nucleotides, 2′-O-methyl modified nucleotides, 2′-O-(2-methoxyethyl) modified nucleotides, deoxyribose nucleotides and/or LNAs at its 5′ terminus, e.g., within the first 5 nucleotides at the 5′ terminus. For example, but not by way of limitation, the first, second and third nucleotides at the 5′ terminus of the gRNA molecule are 2′-fluoro modified nucleotides, 2′-O-methyl modified nucleotides, 2′-O-(2-methoxyethyl) modified nucleotides, deoxyribose nucleotides, LNAs or a combination thereof. In certain embodiments, the first, second and third nucleotides at the 5′ terminus of the gRNA molecule are 2′-fluoro modified nucleotides. In certain embodiments, the first, second and third nucleotides at the 5′ terminus of the gRNA molecule are 2′-O-methyl modified nucleotides. In certain embodiments, the first, second and third nucleotides at the terminus of the gRNA molecule are 2′-O-(2-methoxyethyl) modified nucleotides. In certain embodiments, the first, second and third nucleotides at the 5′ terminus of the gRNA molecule are LNAs. In certain embodiments, the first, second and third nucleotides at the 5′ terminus of the gRNA molecule have a deoxyribose sugar.

In certain embodiments, a gRNA of the present disclosure includes four or more 2′-fluoro modified nucleotides, 2′-O-methyl modified nucleotides, 2′-O-(2-methoxyethyl) modified nucleotides, deoxyribose nucleotides and/or LNAs at its 5′ terminus, e.g., within the first 5 nucleotides at the 5′ terminus. For example, but not by way of limitation, the first, second, third and fourth nucleotides at the 5′ terminus of the gRNA molecule are 2′-fluoro modified nucleotides, 2′-O-methyl modified nucleotides, 2′-O-(2-methoxyethyl) modified nucleotides, deoxyribose nucleotides, LNAs or a combination thereof. In certain embodiments, the first, second, third and fourth nucleotides at the 5′ terminus of the gRNA molecule are 2′-fluoro modified nucleotides. In certain embodiments, the first, second, third and fourth nucleotides at the 5′ terminus of the gRNA molecule are 2′-O-methyl modified nucleotides. In certain embodiments, the first, second, third and fourth nucleotides at the terminus of the gRNA molecule are 2′-O-(2-methoxyethyl) modified nucleotides. In certain embodiments, the first, second, third and fourth at the 5′ terminus of the gRNA molecule are LNAs. In certain embodiments, the first, second, third and fourth nucleotides at the 5′ terminus of the gRNA molecule have a deoxyribose sugar.

In certain embodiments, a gRNA of the present disclosure includes five or more 2′-fluoro modified nucleotides, 2′-O-methyl modified nucleotides, 2′-O-(2-methoxyethyl) modified nucleotides, deoxyribose nucleotides and/or LNAs at its 5′ terminus, e.g., within the first 5 nucleotides at the 5′ terminus. For example, but not by way of limitation, the first, second, third, fourth and fifth nucleotides at the 5′ terminus of the gRNA molecule are 2′-fluoro modified nucleotides, 2′-O-methyl modified nucleotides, 2′-O-(2-methoxyethyl) modified nucleotides, deoxyribose nucleotides, LNAs or a combination thereof. In certain embodiments, the first, second, third, fourth and fifth nucleotides at the 5′ terminus of the gRNA molecule are 2′-fluoro modified nucleotides. In certain embodiments, the first, second, third, fourth and fifth nucleotides at the 5′ terminus of the gRNA molecule are 2′-O-methyl modified nucleotides. In certain embodiments, first, second, third, fourth and fifth nucleotides at the terminus of the gRNA molecule are 2′-O-(2-methoxyethyl) modified nucleotides. In certain embodiments, first, second, third, fourth and fifth nucleotides at the 5′ terminus of the gRNA molecule are LNAs. In certain embodiments, first, second, third, fourth and fifth nucleotides at the 5′ terminus of the gRNA molecule have a deoxyribose sugar.

In certain embodiments, a gRNA of the present disclosure includes one or more of a 2′-fluoro modified nucleotide, a 2′-O-methyl modified nucleotide, a 2′-O-(2-methoxyethyl) modified nucleotide, a deoxyribose nucleotide and/or a locked nucleic acid (LNA) at its 3′ terminus, e.g., within the last 5 nucleotides at the 3′ terminus. For example, but not by way of limitation, the 3′ terminal (“n”) nucleotide of the gRNA molecule is a 2′-fluoro modified nucleotide, a 2′-O-methyl modified nucleotide, a 2′-O-(2-methoxyethyl) modified nucleotide, a deoxyribose nucleotide or a locked nucleic acid (LNA). In certain embodiments, the n nucleotide of the gRNA molecule is a 2′-fluoro modified nucleotide. In certain embodiments, the n nucleotide of the gRNA molecule is a 2′-O-methyl modified nucleotide. In certain embodiments, the n nucleotide of the gRNA molecule is a 2′-O-(2-methoxyethyl) modified nucleotide. In certain embodiments, the n nucleotide of the gRNA molecule is an LNA. In certain embodiments, the n nucleotide of the gRNA molecule has a deoxyribose sugar.

In certain embodiments, a gRNA of the present disclosure includes two or more 2′-fluoro modified nucleotides, 2′-O-methyl modified nucleotides, 2′-O-(2-methoxyethyl) modified nucleotides, deoxyribose nucleotides and/or LNAs at its 3′ terminus, e.g., within the last 5 nucleotides at the 3′ terminus. For example, but not by way of limitation, the n and n−1 nucleotides of the gRNA molecule are 2′-fluoro modified nucleotides, 2′-O-methyl modified nucleotides, 2′-O-(2-methoxyethyl) modified nucleotides, deoxyribose nucleotides, LNAs or a combination thereof. In certain embodiments, the n and n−1 nucleotides of the gRNA molecule are 2′-fluoro modified nucleotides. In certain embodiments, the n and n−1 nucleotides of the guide RNA molecule are 2′-O-methyl modified nucleotides. In certain embodiments, the n and n−1 nucleotides at the terminus of the gRNA molecule are 2′-O-(2-methoxyethyl) modified nucleotides. In certain embodiments, the n and n−1 nucleotides of the gRNA molecule are LNAs. In certain embodiments, the n and n−1 nucleotides of the gRNA molecule have a deoxyribose sugar.

In certain embodiments, a gRNA of the present disclosure includes three or more 2′-fluoro modified nucleotides, 2′-O-methyl modified nucleotides, 2′-O-(2-methoxyethyl) modified nucleotides, deoxyribose nucleotides and/or LNAs at its 3′ terminus, e.g., within the last 5 nucleotides at the 3′ terminus. For example, but not by way of limitation, the n, n−1 and n−2 nucleotides of the gRNA molecule are 2′-fluoro modified nucleotides, 2′-O-methyl modified nucleotides, 2′-O-(2-methoxyethyl) modified nucleotides, deoxyribose nucleotides, LNAs or a combination thereof. In certain embodiments, the n, n−1 and n−2 nucleotides of the gRNA molecule are 2′-fluoro modified nucleotides. In certain embodiments, the n, n−1 and n−2 nucleotides of the guide RNA molecule are 2′-O-methyl modified nucleotides. In certain embodiments, the n, n−1 and n−2 nucleotides at the terminus of the gRNA molecule are 2′-O-(2-methoxyethyl) modified nucleotides. In certain embodiments, the n, n−1 and n−2 nucleotides of the gRNA molecule are LNAs. In certain embodiments, the n, n−1 and n−2 nucleotides of the gRNA molecule have a deoxyribose sugar.

In certain embodiments, a gRNA of the present disclosure includes four or more 2′-fluoro modified nucleotides, 2′-O-methyl modified nucleotides, 2′-O-(2-methoxyethyl) modified nucleotides, deoxyribose nucleotides and/or LNAs at its 3′ terminus, e.g., within the last 5 nucleotides at the 3′ terminus. For example, but not by way of limitation, the n, n−1, n−2 and n−3 nucleotides of the gRNA molecule are 2′-fluoro modified nucleotides, 2′-O-methyl modified nucleotides, 2′-O-(2-methoxyethyl) modified nucleotides, deoxyribose nucleotides, LNAs or a combination thereof. In certain embodiments, the n−1, n−2, n−3 and n−4 nucleotides of the gRNA molecule are 2′-fluoro modified nucleotides, 2′-O-methyl modified nucleotides, 2′-O-(2-methoxyethyl) modified nucleotides, LNAs or a combination thereof. In certain embodiments, the n, n−1, n−2 and n−3 nucleotides of the gRNA molecule are 2′-fluoro modified nucleotides. In certain embodiments, the n, n−1, n−2 and n−3 nucleotides of the guide RNA molecule are 2′-O-methyl modified nucleotides. In certain embodiments, the n, n−1, n−2 and n−3 nucleotides at the terminus of the gRNA molecule are 2′-O-(2-methoxyethyl) modified nucleotides. In certain embodiments, the n, n−1, n−2 and n−3 nucleotides of the gRNA molecule are LNAs. In certain embodiments, the n, n−1, n−2 and n−3 nucleotides of the gRNA molecule have a deoxyribose sugar. In certain embodiments, the n−1, n−2, n−3 and n−4 nucleotides of the gRNA molecule are 2′-O-methyl modified nucleotides.

In certain embodiments, a gRNA of the present disclosure includes five or more 2′-fluoro modified nucleotides, 2′-O-methyl modified nucleotides, 2′-O-(2-methoxyethyl) modified nucleotides, deoxyribose nucleotides and/or LNAs at its 3′ terminus, e.g., within the last 5 nucleotides at the 3′ terminus. For example, but not by way of limitation, the n, n−1, n−2, n−3 and n−4 nucleotides of the gRNA molecule are 2′-fluoro modified nucleotides, 2′-O-methyl modified nucleotides, 2′-O-(2-methoxyethyl) modified nucleotides, deoxyribose nucleotides, LNAs or a combination thereof. In certain embodiments, the n, n−1, n−2, n−3 and n−4 nucleotides of the gRNA molecule are 2′-fluoro modified nucleotides. In certain embodiments, the n, n−1, n−2, n−3 and n−4 nucleotides of the guide RNA molecule are 2′-O-methyl modified nucleotides. In certain embodiments, the n, n−1, n−2, n−3 and n−4 nucleotides at the terminus of the gRNA molecule are 2′-O-(2-methoxyethyl) modified nucleotides. In certain embodiments, the n, n−1, n−2, n−3 and n−4 nucleotides of the gRNA molecule are LNAs. In certain embodiments, the n, n−1, n−2, n−3 and n−4 nucleotides of the gRNA molecule have a deoxyribose sugar.

In certain embodiments, a gRNA of the present disclosure includes (i) one or more 2′-fluoro modified nucleotides, 2′-O-methyl modified nucleotides, 2′-O-(2-methoxyethyl) modified nucleotides, deoxyribose nucleotide and/or LNAs at its 5′ terminus and (ii) one or more phosphorodithioate linkages at the 5′ terminus of the gRNA molecule. In certain embodiments, the first nucleotide at 5′ terminus of the gRNA molecule is a 2′-fluoro modified nucleotide, a 2′-O-methyl modified nucleotide, a 2′-O-(2-methoxyethyl) modified nucleotide, deoxyribose nucleotide or a locked nucleic acid (LNA) and the gRNA includes a phosphorodithioate linkage between the first and second nucleotides at the 5′ terminus of the gRNA molecule. In certain embodiments, the first nucleotide at 5′ terminus of the gRNA molecule is a 2′-O-methyl modified nucleotide and the gRNA includes a phosphorodithioate linkage between the first and second nucleotides at the 5′ terminus of the gRNA molecule.

In certain embodiments, a gRNA of the present disclosure includes (i) two or more 2′-fluoro modified nucleotides, 2′-O-methyl modified nucleotides, 2′-O-(2-methoxyethyl) modified nucleotides, deoxyribose nucleotides and/or LNAs at its 5′ terminus and (ii) two or more phosphorodithioate linkages at the 5′ terminus of the gRNA molecule. In certain embodiments, the first and second nucleotides at the 5′ terminus of the gRNA molecule are 2′-fluoro modified nucleotides, 2′-O-methyl modified nucleotides, 2′-O-(2-methoxyethyl) modified nucleotides, deoxyribose nucleotides or LNAs and the gRNA molecule includes a phosphorodithioate linkage between the first and second nucleotides and the second and third nucleotides at the 5′ terminus of the gRNA molecule. In certain embodiments, the first and second nucleotides at the 5′ terminus of the gRNA molecule are 2′-O-methyl modified nucleotides and the gRNA molecule includes phosphorodithioate linkages between the first and second nucleotides and the second and third nucleotides at the 5′ terminus of the gRNA molecule.

In certain embodiments, a gRNA of the present disclosure includes (i) three or more 2′-fluoro modified nucleotides, 2′-O-methyl modified nucleotides, 2′-O-(2-methoxyethyl) modified nucleotides, deoxyribose nucleotides and/or LNAs at its 5′ terminus and (ii) three or more phosphorodithioate linkages at the 5′ terminus of the gRNA molecule. In certain embodiments, the first, second and third nucleotides at the 5′ terminus of the gRNA molecule are 2′-fluoro modified nucleotides, 2′-O-methyl modified nucleotides, 2′-O-(2-methoxyethyl) modified nucleotides, deoxyribose nucleotides or LNAs and the gRNA includes phosphorodithioate linkages between the first and second nucleotides, the second and third nucleotides and the third and fourth nucleotides at the 5′ terminus of the gRNA molecule. In certain embodiments, the first, second and third nucleotides at the 5′ terminus of the gRNA molecule are 2′-O-methyl modified nucleotides and the gRNA includes phosphorodithioate linkages between the first and second nucleotides, the second and third nucleotides and the third and fourth nucleotides at the 5′ terminus of the gRNA molecule.

In certain embodiments, a gRNA of the present disclosure includes (i) four or more 2′-fluoro modified nucleotides, 2′-O-methyl modified nucleotides, 2′-O-(2-methoxyethyl) modified nucleotides, deoxyribose nucleotides and/or LNAs at its 5′ terminus and (ii) four or more phosphorodithioate linkages at the 5′ terminus of the gRNA molecule. In certain embodiments, the first, second, third and fourth nucleotides at the 5′ terminus of the gRNA molecule are 2′-fluoro modified nucleotides, 2′-O-methyl modified nucleotides, 2′-O-(2-methoxyethyl) modified nucleotides, deoxyribose nucleotides or LNAs and the gRNA includes phosphorodithioate linkages between the first and second nucleotides, the second and third nucleotides, the third and fourth nucleotides and fourth and fifth nucleotides at the 5′ terminus of the gRNA molecule. In certain embodiments, the first, second, third and fourth nucleotides at the 5′ terminus of the gRNA molecule are 2′-O-methyl modified nucleotides and the gRNA includes phosphorodithioate linkages between the first and second nucleotides, the second and third nucleotides, the third and fourth nucleotides and fourth and fifth nucleotides at the 5′ terminus of the gRNA molecule.

In certain embodiments, a gRNA of the present disclosure includes (i) one or more 2′-fluoro modified nucleotides, 2′-O-methyl modified nucleotides, 2′-O-(2-methoxyethyl) modified nucleotides, deoxyribose nucleotides and/or LNAs at its 3′ terminus and (ii) one or more phosphorodithioate linkages at the 3′ terminus of the gRNA molecule. In certain embodiments, the n nucleotide of the gRNA molecule is a 2′-fluoro modified nucleotide, a 2′-O-methyl modified nucleotide, a 2′-O-(2-methoxyethyl) modified nucleotide, deoxyribose nucleotides or a locked nucleic acid (LNA) and the gRNA includes a phosphorodithioate linkage between the n and n−1 nucleotides of the gRNA molecule. In certain embodiments, the n nucleotide of the gRNA molecule is a 2′-O-methyl modified nucleotide and the gRNA includes a phosphorodithioate linkage between the n and n−1 nucleotides of the gRNA molecule.

In certain embodiments, a gRNA of the present disclosure includes (i) two or more 2′-fluoro modified nucleotides, 2′-O-methyl modified nucleotides, 2′-O-(2-methoxyethyl) modified nucleotides, deoxyribose nucleotides and/or LNAs at its 3′ terminus and (ii) two or more phosphorodithioate linkages at the 3′ terminus of the gRNA molecule. In certain embodiments, the n and n−1 or n and n−2 nucleotides of the gRNA molecule are 2′-fluoro modified nucleotides, 2′-O-methyl modified nucleotides, 2′-O-(2-methoxyethyl) modified nucleotides or LNAs and the gRNA molecule includes a phosphorodithioate linkage between the n and n−1 nucleotides and the n−1 and n−2 nucleotides or between the n and n−1 nucleotides and the n−2 and n−3 nucleotides of the gRNA molecule. In certain embodiments, the n and n−1 nucleotides or the n and n−2 nucleotides of the gRNA molecule are 2′-O-methyl modified nucleotides and the gRNA molecule includes a phosphorodithioate linkage between the n and n−1 nucleotides and the n−1 and n−2 nucleotides or between the n and n−1 nucleotides and the n−2 and n−3 nucleotides of the gRNA molecule.

In certain embodiments, a gRNA of the present disclosure includes (i) three or more 2′-fluoro modified nucleotides, 2′-O-methyl modified nucleotides, 2′-O-(2-methoxyethyl) modified nucleotides, deoxyribose nucleotides and/or LNAs at its 3′ terminus and (ii) two or more phosphorodithioate linkages at the 3′ terminus of the gRNA molecule. In certain embodiments, the n, n−1 and n−2 nucleotides the gRNA molecule are 2′-fluoro modified nucleotides, 2′-O-methyl modified nucleotides, 2′-O-(2-methoxyethyl) modified nucleotides, deoxyribose nucleotides or LNAs and the gRNA molecule includes a phosphorodithioate linkage between the n and n−1 nucleotides and the n−1 and n−2 nucleotides or between the n and n−1 nucleotides and the n−2 and n−3 nucleotides of the gRNA molecule. In certain embodiments, the n, n−1 and n−2 nucleotides the gRNA molecule are 2′-O-methyl modified nucleotides and the gRNA molecule includes a phosphorodithioate linkage between the n and n−1 nucleotides and the n−1 and n−2 nucleotides or between the n and n−1 nucleotides and the n−2 and n−3 nucleotides of the gRNA molecule. In certain embodiments, the n, n−1 and n−2 nucleotides the gRNA molecule are 2′-O-methyl modified nucleotides and the gRNA molecule includes a phosphorodithioate linkage between the n and n−1 nucleotides and the n−1 and n−2 nucleotides or between the n and n−1 nucleotides of the gRNA molecule.

In certain embodiments, a gRNA of the present disclosure includes (i) four or more 2′-fluoro modified nucleotides, 2′-O-methyl modified nucleotides, 2′-O-(2-methoxyethyl) modified nucleotides, deoxyribose nucleotides and/or LNAs at its 3′ terminus and (ii) four or more phosphorodithioate linkages at the 3′ terminus of the gRNA molecule. In certain embodiments, the n, n−1, n−2 and n−3 nucleotides the gRNA molecule are 2′-fluoro modified nucleotides, 2′-O-methyl modified nucleotides, 2′-O-(2-methoxyethyl) modified nucleotides, deoxyribose nucleotides or LNAs and the gRNA molecule includes a phosphorodithioate linkage between the n and n−1 nucleotides, between the n−1 and n−2 nucleotides, between the n−2 and n−3 nucleotides and between n−3 and n−4 nucleotides of the gRNA molecule. In certain embodiments, the n, n−1, n−2 and n−3 nucleotides the gRNA molecule are 2′-O-methyl modified nucleotides and the gRNA molecule includes a phosphorodithioate linkage between the n and n−1 nucleotides, between the n−1 and n−2 nucleotides, between the n−2 and n−3 nucleotides and between n−3 and n−4 nucleotides of the gRNA molecule.

In certain embodiments, a gRNA of the present disclosure includes (i) five or more 2′-fluoro modified nucleotides, 2′-O-methyl modified nucleotides, 2′-O-(2-methoxyethyl) modified nucleotides, deoxyribose nucleotides and/or LNAs at its 3′ terminus and (ii) four or more phosphorodithioate linkages at the 3′ terminus of the gRNA molecule. In certain embodiments, the n, n−1, n−2, n−3 and n−4 nucleotides the gRNA molecule are 2′-fluoro modified nucleotides, 2′-O-methyl modified nucleotides, 2′-O-(2-methoxyethyl) modified nucleotides, deoxyribose nucleotides or LNAs and the gRNA molecule includes a phosphorodithioate linkage between the n and n−1 nucleotides, between the n−1 and n−2 nucleotides, between the n−2 and n−3 nucleotides and between n−3 and n−4 nucleotides of the gRNA molecule. In certain embodiments, the n, n−1, n−2, n−3 and n−4 nucleotides the gRNA molecule are 2′-O-methyl modified nucleotides and the gRNA molecule includes a phosphorodithioate linkage between the n and n−1 nucleotides, between the n−1 and n−2 nucleotides, between the n−2 and n−3 nucleotides and between n−3 and n−4 nucleotides of the gRNA molecule.

In certain embodiments, a gRNA of the present disclosure includes (i) one or more 2′-fluoro modified nucleotides, 2′-O-methyl modified nucleotides, 2′-O-(2-methoxyethyl) modified nucleotides, deoxyribose nucleotides and/or LNAs at the 5′ terminus, (ii) one or more phosphorodithioate linkages at the 5′ terminus, (iii) one or more 2′-fluoro modified nucleotides, 2′-O-methyl modified nucleotides, 2′-O-(2-methoxyethyl) modified nucleotides, deoxyribose nucleotides and LNAs at the 3′ terminus and (iv) one or more phosphorodithioate linkages at the 3′ terminus. In certain embodiments, a gRNA of the present disclosure includes (i) one or more 2′-O-methyl modified nucleotides at the 5′ terminus, (ii) one or more phosphorodithioate linkages at the 5′ terminus, (iii) one or more 2′-O-methyl modified nucleotides at the 3′ terminus and (iv) one or more phosphorodithioate linkages at the 3′ terminus.

In certain embodiments, a gRNA of the present disclosure includes (i) two or more 2′-fluoro modified nucleotides, 2′-O-methyl modified nucleotides, 2′-O-(2-methoxyethyl) modified nucleotides, deoxyribose nucleotides and/or LNAs at the 5′ terminus, (ii) two or more phosphorodithioate linkages at the 5′ terminus, (iii) two or more 2′-fluoro modified nucleotides, 2′-O-methyl modified nucleotides, 2′-O-(2-methoxyethyl) modified nucleotides, deoxyribose nucleotides and LNAs at the 3′ terminus and (iv) two or more phosphorodithioate linkages at the 3′ terminus. In certain embodiments, a gRNA of the present disclosure includes (i) two or more 2′-O-methyl modified nucleotides at the 5′ terminus, (ii) two or more phosphorodithioate linkages at the 5′ terminus, (iii) two or more 2′-O-methyl modified nucleotides at the 3′ terminus and (iv) two or more phosphorodithioate linkages at the 3′ terminus.

In certain embodiments, a gRNA of the present disclosure includes (i) three or more 2′-fluoro modified nucleotides, 2′-O-methyl modified nucleotides, 2′-O-(2-methoxyethyl) modified nucleotides, deoxyribose nucleotides and/or LNAs at the 5′ terminus, (ii) two or more phosphorodithioate linkages at the 5′ terminus, (iii) three or more 2′-fluoro modified nucleotides, 2′-O-methyl modified nucleotides, 2′-O-(2-methoxyethyl) modified nucleotides, deoxyribose nucleotides and LNAs at the 3′ terminus and (iv) two or more phosphorodithioate linkages at the 3′ terminus. In certain embodiments, a gRNA of the present disclosure includes (i) three or more 2′-O-methyl modified nucleotides at the 5′ terminus, (ii) two or more phosphorodithioate linkages at the 5′ terminus, (iii) three or more 2′-O-methyl modified nucleotides at the 3′ terminus and (iv) two or more phosphorodithioate linkages at the 3′ terminus.

In certain embodiments, a gRNA of the present disclosure includes (i) three or more 2′-fluoro modified nucleotides, 2′-O-methyl modified nucleotides, 2′-O-(2-methoxyethyl) modified nucleotides, deoxyribose nucleotides and/or LNAs at the 5′ terminus, (ii) three or more phosphorodithioate linkages at the 5′ terminus, (iii) three or more 2′-fluoro modified nucleotides, 2′-O-methyl modified nucleotides, 2′-O-(2-methoxyethyl) modified nucleotides, deoxyribose nucleotides and LNAs at the 3′ terminus and (iv) one or more phosphorodithioate linkages at the 3′ terminus. In certain embodiments, a gRNA of the present disclosure includes (i) three or more 2′-O-methyl modified nucleotides at the 5′ terminus, (ii) three or more phosphorodithioate linkages at the 5′ terminus, (iii) three or more 2′-O-methyl modified nucleotides at the 3′ terminus and (iv) one or more phosphorodithioate linkages at the 3′ terminus.

In certain embodiments, a gRNA of the present disclosure includes (i) three or more 2′-fluoro modified nucleotides, 2′-O-methyl modified nucleotides, 2′-O-(2-methoxyethyl) modified nucleotides, deoxyribose nucleotides and/or LNAs at the 5′ terminus, (ii) three or more phosphorodithioate linkages at the 5′ terminus, (iii) three or more 2′-fluoro modified nucleotides, 2′-O-methyl modified nucleotides, 2′-O-(2-methoxyethyl) modified nucleotides, deoxyribose nucleotides and LNAs at the 3′ terminus and (iv) two or more phosphorodithioate linkages at the 3′ terminus. In certain embodiments, a gRNA of the present disclosure includes (i) three or more 2′-O-methyl modified nucleotides at the 5′ terminus, (ii) three or more phosphorodithioate linkages at the 5′ terminus, (iii) three or more 2′-O-methyl modified nucleotides at the 3′ terminus and (iv) two or more phosphorodithioate linkages at the 3′ terminus.

In certain embodiments, a gRNA of the present disclosure includes (i) four or more 2′-fluoro modified nucleotides, 2′-O-methyl modified nucleotides, 2′-O-(2-methoxyethyl) modified nucleotides, deoxyribose nucleotides and/or LNAs at the 5′ terminus, (ii) four or more phosphorodithioate linkages at the 5′ terminus, (iii) four or more 2′-fluoro modified nucleotides, 2′-O-methyl modified nucleotides, 2′-O-(2-methoxyethyl) modified nucleotides, deoxyribose nucleotides and LNAs at the 3′ terminus and (iv) four or more phosphorodithioate linkages at the 3′ terminus. In certain embodiments, a gRNA of the present disclosure includes (i) four or more 2′-O-methyl modified nucleotides at the 5′ terminus, (ii) four or more phosphorodithioate linkages at the 5′ terminus, (iii) four or more 2′-O-methyl modified nucleotides at the 3′ terminus and (iv) four or more phosphorodithioate linkages at the 3′ terminus.

In certain embodiments, a gRNA of the present disclosure includes (i) four or more 2′-fluoro modified nucleotides, 2′-O-methyl modified nucleotides, 2′-O-(2-methoxyethyl) modified nucleotides, deoxyribose nucleotides and LNAs at the 5′ terminus, (ii) four or more phosphorodithioate linkages at the 5′ terminus, (iii) five or more 2′-fluoro modified nucleotides, 2′-O-methyl modified nucleotides, 2′-O-(2-methoxyethyl) modified nucleotides, deoxyribose nucleotides and LNAs at the 3′ terminus and (iv) four or more phosphorodithioate linkages at the 3′ terminus. In certain embodiments, a gRNA of the present disclosure includes (i) four or more 2′-O-methyl modified nucleotides at the 5′ terminus, (ii) four or more phosphorodithioate linkages at the 5′ terminus, (iii) five or more 2′-O-methyl modified nucleotides at the 3′ terminus and (iv) four or more phosphorodithioate linkages at the 3′ terminus.

In certain embodiments, a gRNA of the present disclosure includes (i) a 2′-O-methyl modified nucleotide at the 5′ terminus (e.g., first nucleotide), (ii) one phosphorodithioate linkage at the 5′ terminus (e.g., between the first and second nucleotides), (iii) one 2′-O-methyl modified nucleotide at the 3′ terminus (e.g., n nucleotide) and (iv) one phosphorodithioate linkages at the 3′ terminus (e.g., between the n and n−1 nucleotides).

In certain embodiments, a gRNA of the present disclosure includes (i) two 2′-O-methyl modified nucleotides at the 5′ terminus (e.g., first and second nucleotides), (ii) two phosphorodithioate linkages at the 5′ terminus (e.g., between the first and second nucleotides and between the second and third nucleotides), (iii) two 2′-O-methyl modified nucleotides (e.g., the n and n−1 nucleotides) at the 3′ terminus and (iv) two phosphorodithioate linkages at the 3′ terminus (e.g., between the n and n−1 nucleotides and between the n−1 and n−2 nucleotides).

In certain embodiments, a gRNA of the present disclosure includes (i) three 2′-O-methyl modified nucleotides (e.g., first, second and third nucleotides) at the 5′ terminus, (ii) three phosphorodithioate linkages at the 5′ terminus (e.g., between the first and second nucleotides, between the second and third nucleotides and between the third and fourth nucleotides), (iii) three 2′-O-methyl modified nucleotides (e.g., the n, n−1 and n−2 nucleotides) at the 3′ terminus and (iv) two phosphorodithioate linkages at the 3′ terminus (e.g., between the n and n−1 nucleotides and between the n−1 and n−2 nucleotides).

In certain embodiments, a gRNA of the present disclosure includes (i) three 2′-O-methyl modified nucleotides (e.g., first, second and third nucleotides) at the 5′ terminus, (ii) three phosphorothioate linkages at the 5′ terminus (e.g., between the first and second nucleotides, between the second and third nucleotides and between the third and fourth nucleotides), (iii) three 2′-O-methyl modified nucleotides (e.g., the n, n−1 and n−2 nucleotides) at the 3′ terminus and (iv) two phosphorodithioate linkages at the 3′ terminus (e.g., between the n and n−1 nucleotides and between the n−1 and n−2 nucleotides).

In certain embodiments, a gRNA of the present disclosure includes (i) three 2′-O-methyl modified nucleotides (e.g., first, second and third nucleotides) at the 5′ terminus, (ii) two phosphorodithioate linkages at the 5′ terminus (e.g., between the first and second nucleotides and between the second and third nucleotides or between second and third nucleotides and the third and fourth nucleotides), (iii) three 2′-O-methyl modified nucleotides (e.g., the n, n−1 and n−2 nucleotides) at the 3′ terminus and (iv) two phosphorodithioate linkages at the 3′ terminus (e.g., between the n and n−1 nucleotides and between the n−1 and n−2 nucleotides or between the n−1 and n−2 nucleotides and between the n−2 and n−3 nucleotides).

In certain embodiments, a gRNA of the present disclosure includes (i) three 2′-O-methyl modified nucleotides (e.g., first, second and third nucleotides) at the 5′ terminus, (ii) two phosphorodithioate linkages at the 5′ terminus (e.g., between the first and second nucleotides and between the second and third nucleotides or between second and third nucleotides and the third and fourth nucleotides), (iii) one phosphorothioate linkage at the 5′ terminus (e.g., between the first and second nucleotides, between the second and third nucleotides or between the third and fourth nucleotides), (iv) three 2′-O-methyl modified nucleotides (e.g., the n, n−1 and n−2 nucleotides) at the 3′ terminus and (v) two phosphorodithioate linkages at the 3′ terminus (e.g., between the n and n−1 nucleotides and between the n−1 and n−2 nucleotides or between the n−1 and n−2 nucleotides and between the n−2 and n−3 nucleotides).

In certain embodiments, a gRNA of the present disclosure includes (i) three 2′-O-methyl modified nucleotides (e.g., first, second and third nucleotides) at the 5′ terminus, (ii) three phosphorodithioate linkages at the 5′ terminus (e.g., between the first and second nucleotides, between the second and third nucleotides and between the third and fourth nucleotides), (iii) three 2′-O-methyl modified nucleotides (e.g., the n, n−1 and n−2 nucleotides) at the 3′ terminus and (iv) one phosphorodithioate linkages at the 3′ terminus (e.g., between the n and n−1 nucleotides, between the n−1 and n−2 nucleotides or between the n−2 and n−3 nucleotides).

In certain embodiments, a gRNA of the present disclosure includes (i) three 2′-O-methyl modified nucleotides (e.g., first, second and third nucleotides) at the 5′ terminus, (ii) three phosphorodithioate linkages at the 5′ terminus (e.g., between the first and second nucleotides, between the second and third nucleotides and between the third and fourth nucleotides), (iii) three 2′-O-methyl modified nucleotides (e.g., the n, n−1 and n−2 nucleotides) at the 3′ terminus, (iv) one phosphorodithioate linkage at the 3′ terminus (e.g., between the n and n−1 nucleotides, between the n−1 and n−2 nucleotides or between the n−2 and n−3 nucleotides) and (v) one phosphorothioate linkage at the 3′ terminus (e.g., between the n and n−1 nucleotides, between the n−1 and n−2 nucleotides or between the n−2 and n−3 nucleotides).

In certain embodiments, a gRNA of the present disclosure includes (i) four 2′-O-methyl modified nucleotides (e.g., first, second, third and fourth nucleotides) at the 5′ terminus, (ii) four phosphorodithioate linkages at the 5′ terminus (e.g., between the first and second nucleotides, between the second and third nucleotides, between the third and fourth nucleotides and between the fourth and fifth nucleotides), (iii) four 2′-O-methyl modified nucleotides (e.g., the n, n−1, n−2 and n−3 nucleotides) at the 3′ terminus and (iv) four phosphorodithioate linkages at the 3′ terminus (e.g., between the n and n−1 nucleotides, between the n−1 and n−2 nucleotides, between the n−2 and n−3 nucleotides and between the n−3 and n−4 nucleotides).

In certain embodiments, a gRNA of the present disclosure includes (i) four 2′-O-methyl modified nucleotides (e.g., first, second, third and fourth nucleotides) at the 5′ terminus, (ii) four phosphorodithioate linkages at the 5′ terminus (e.g., between the first and second nucleotides, between the second and third nucleotides, between the third and fourth nucleotides and between the fourth and fifth nucleotides), (iii) five 2′-O-methyl modified nucleotides (e.g., the n, n−1, n−2, n−3 and n−4 nucleotides) at the 3′ terminus and (iv) four phosphorodithioate linkages at the 3′ terminus (e.g., between the n and n−1 nucleotides, between the n−1 and n−2 nucleotides, between the n−2 and n−3 nucleotides and between the n−3 and n−4 nucleotides).

In certain embodiments, a gRNA of the present disclosure includes (i) four 2′-O-methyl modified nucleotides (e.g., first, second, third and fourth nucleotides) at the 5′ terminus, (ii) four phosphorodithioate linkages at the 5′ terminus (e.g., between the first and second nucleotides, between the second and third nucleotides, between the third and fourth nucleotides and between the fourth and fifth nucleotides), (iii) five 2′-O-methyl modified nucleotides (e.g., the n, n−1, n−2, n−3 and n−4 nucleotides) at the 3′ terminus and (iv) four phosphorodithioate linkages at the 3′ terminus (e.g., between the n−1 and n−2 nucleotides, between the n−2 and n−3 nucleotides, between the n−3 and n−4 nucleotides and between the n−4 and n−5 nucleotides).

In certain embodiments, a modified gRNA of the present disclosure can be modified according to a modification scheme disclosed in Tables 1 and 10. For example, but not by way of limitation, a modified gRNA can be modified according to modification scheme “A” of Table 1. In certain embodiments, a modified gRNA can be modified according to modification scheme “A” of Table 1. In certain embodiments, a modified gRNA can be modified according to modification scheme “B” of Table 1. In certain embodiments, a modified gRNA can be modified according to modification scheme “C” of Table 1. In certain embodiments, a modified gRNA can be modified according to modification scheme “D” of Table 1. In certain embodiments, a modified gRNA can be modified according to modification scheme “E” of Table 1. In certain embodiments, a modified gRNA can be modified according to modification scheme “F” of Table 1. In certain embodiments, a modified gRNA can be modified according to modification scheme “G” of Table 1. In certain embodiments, a modified gRNA can be modified according to modification scheme “H” of Table 1. In certain embodiments, a modified gRNA can be modified according to modification scheme “I” of Table 1. In certain embodiments, a modified gRNA can be modified according to modification scheme “J” of Table 1. In certain embodiments, a modified gRNA can be modified according to modification scheme “K” of Table 1. In certain embodiments, a modified gRNA can be modified according to modification scheme “L” of Table 1. In certain embodiments, a modified gRNA can be modified according to modification scheme “M” of Table 10. In certain embodiments, a modified gRNA can be modified according to modification scheme “N” of Table 10. In certain embodiments, a modified gRNA can be modified according to modification scheme “O” of Table 10. In certain embodiments, a modified gRNA can be modified according to modification scheme “P” of Table 10. In certain embodiments, a modified gRNA can be modified according to modification scheme “Q” of Table 10. In certain embodiments, a modified gRNA can be modified according to modification scheme “R” of Table 10. In certain embodiments, a modified gRNA can be modified according to modification scheme “S” of Table 10. In certain embodiments, a modified gRNA can be modified according to modification scheme “T” of Table 10. In certain embodiments, a modified gRNA can be modified according to modification scheme “U” of Table 10. In certain embodiments, a modified gRNA can be modified according to modification scheme “V” of Table 10.

In certain embodiments, a modified gRNA of the present disclosure can have an editing efficiency of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100%.

In certain embodiments, a modified gRNA of the present disclosure can have an editing efficiency of about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater or about 95% or greater. In certain embodiments, a modified gRNA of the present disclosure has an editing efficiency of about 60% or greater. In certain embodiments, a modified gRNA of the present disclosure has an editing efficiency of about 65% or greater. In certain embodiments, a modified gRNA of the present disclosure has an editing efficiency of about 70% or greater. In certain embodiments, a modified gRNA of the present disclosure has an editing efficiency of about 75% or greater. In certain embodiments, a modified gRNA of the present disclosure has an editing efficiency of about 80% or greater. In certain embodiments, a modified gRNA of the present disclosure has an editing efficiency of about 85% or greater. In certain embodiments, a modified gRNA of the present disclosure has an editing efficiency of about 90% or greater. In certain embodiments, a modified gRNA of the present disclosure has an editing efficiency of about 95% or greater.

In certain embodiments, a modified gRNA of the present disclosure can have an editing efficiency from about 5% to about 100%. For example, but not by way of limitation, a modified gRNA of the present disclosure can have an editing efficiency from about 5% to about 100%, about 10% to about 100%, about 20% to about 100%, about 30% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100% or about 95% to about 100%. In certain embodiments, a modified gRNA of the present disclosure can have an editing efficiency from about 50% to about 100%. In certain embodiments, a modified gRNA of the present disclosure can have an editing efficiency from about 60% to about 100%. In certain embodiments, a modified gRNA of the present disclosure can have an editing efficiency from about 70% to about 100%. In certain embodiments, a modified gRNA of the present disclosure can have an editing efficiency from about 80% to about 100%. In certain embodiments, a modified gRNA of the present disclosure can have an editing efficiency from about 90% to about 100%.

In certain embodiments, a modified gRNA of the present disclosure can retain the editing efficiency of a reference gRNA or have improved editing efficiency compared to a reference gRNA. For example, but not by way of limitation, a modified gRNA of the present disclosure can retain about 50% or more, about 55% or more, about 60% or more, about 65% or more, about 70% or more, about 75% or more, about 80% or more, about 85% or more, about 90% or more or about 95% or more of the editing efficiency of a reference gRNA. In certain embodiments, a modified gRNA of the present disclosure can retain about 50% or more of the editing efficiency of a reference gRNA. In certain embodiments, a modified gRNA of the present disclosure can retain about 60% or more of the editing efficiency of a reference gRNA. In certain embodiments, a modified gRNA of the present disclosure can retain about 70% or more of the editing efficiency of a reference gRNA. In certain embodiments, a modified gRNA of the present disclosure can retain about 80% or more of the editing efficiency of a reference gRNA. In certain embodiments, a modified gRNA of the present disclosure can retain about 90% or more of the editing efficiency of a reference gRNA. In certain embodiments, a modified gRNA of the present disclosure can retain from about 50% to about 100% of the editing efficiency of a reference gRNA, e.g., from about 55% to about 100%, about 60% to about 100%, about 65% to about 100%, about 70% to about 100%, about 75% to about 100%, about 80% to about 100%, about 85% to about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100% or about 99% to about 100%. In certain embodiments, a modified gRNA of the present disclosure can retain from about 60% to about 100% of the editing efficiency of a reference gRNA. In certain embodiments, a modified gRNA of the present disclosure can retain from about 70% to about 100% of the editing efficiency of a reference gRNA. In certain embodiments, a modified gRNA of the present disclosure can retain from about 80% to about 100% of the editing efficiency of a reference gRNA. In certain embodiments, a modified gRNA of the present disclosure can retain from about 90% to about 100% of the editing efficiency of a reference gRNA. In certain embodiments, a modified gRNA of the present disclosure can exhibit an increase in editing efficiency compared to a reference gRNA, e.g., an increase in editing efficiency of about 1% or more, about 2% or more, about 3% or more, about 4% or more, about 5% or more, about 6% or more, about 7% or more, about 8% or more, about 9% or more or about 10% or more. In certain embodiments, a modified gRNA of the present disclosure can exhibit an increase in editing efficiency from about 1% to about 10%. In certain embodiments, the reference gRNA can be a gRNA that has the same nucleotide sequence as the modified gRNA (e.g., without the same modifications as the modified gRNA). In certain embodiments, the reference gRNA can be a gRNA that has a targeting domain with the same sequence as the modified gRNA (e.g., without the same modifications as the modified gRNA). In certain embodiments, the reference gRNA can be a gRNA that has the same number of nucleotides as the modified gRNA (e.g., without the same modifications as the modified gRNA).

### III. RNA-GUIDED NUCLEASES

RNA-guided nucleases of a variety of types and species can be used in the present disclosure. The RNA-guided nucleases are for use in combination with one or more modified gRNAs disclosed herein, e.g., in Section II.

In certain embodiments, the RNA-guided nuclease is a Cas protein. Non-limiting examples of Cas proteins are disclosed in Makarova and Koonin, Methods Mol. Biol. 1311:47-75 (2015), the contents of which are incorporated herein by reference in their entirety. In certain embodiments, the Cas proteins include Cas1, Cas2, Cas3, Cas3-HD, Cas4, Cas5, Cas6, Cas7, Cas8, Cas9, Cas10, Cas12a (Cpf1), Cas13, Cmr1, Cmr3, Cmr4, Cmr5, Cmr6, Csy1, Csy2, Csy3, Cse1, Cse2, Csc1, Csc2, Csa5, CsaX, Csm2, Csm3, Csm4, Csm5, Csm6, Csn2, Csb1, Csb2, Csb3, Csx1, Csx3, Csx10, Csx14, Csx15, Csx16, Csx17, Csf1, Csf2, Csf3, Csf4, C2c1, C2c2 and C2c3. In certain embodiments, the Cas protein is selected from the group consisting of a Cas9, a Cas12a, a Cas13 and a combination thereof. In certain embodiments, the Cas protein is a Cas9 protein.

In certain embodiments, the Cas protein is an engineered Cas protein that differs from a reference Cas protein, e.g., a wild-type Cas protein. In certain embodiments, the reference Cas protein is a naturally occurring Cas protein. In certain embodiments, the Cas protein includes one or more amino acid variations compared to a reference Cas protein, e.g., a wild-type Cas protein. In certain embodiments, an engineered Cas protein retains or substantially retains the nuclease (e.g., endonuclease) activity of the reference Cas protein. In certain embodiments, the engineered Cas protein retains at least about 70%, about 80%, about 90%, about 95% or about 99% nuclease activity of the reference Cas protein. In certain embodiments, an engineered Cas protein has no or no substantial cleavage activity. In certain embodiments, a Cas protein can lack cleavage activity or have substantially less, e.g., less than 20%, about 10%, about 5% or about 1% of the cleavage activity of a reference Cas protein.

In certain embodiments, the engineered Cas protein includes one or more deletions that reduces the size of the Cas protein while at least partially retaining the nuclease activity of the Cas protein. In certain embodiments, the reduced size of the engineered Cas protein can allow flexibility with respect to the methods for delivering such engineered Cas proteins.

In certain embodiments, a Cas protein interacts with a gRNA molecule of the present disclosure and, in concert with the gRNA molecule, localizes to a target nucleic acid that includes a target sequence (e.g., a sequence that is complementary to a sequence of the gRNA molecule) and a PAM sequence. In certain embodiments, the ability of a Cas protein to interact with and cleave a target nucleic acid is PAM sequence dependent. In certain embodiments, cleavage of the target nucleic acid occurs upstream from the PAM sequence. In certain embodiments, cleavage of the target nucleic acid occurs downstream from the PAM sequence. Cas molecules from different species, e.g., bacterial species, can recognize different PAM sequences.

In certain embodiments, a Cas protein for use in the present disclosure can be derived from any one of the following species: Streptococcus pyogenes, Streptococcus pneumoniae, Streptococcus thermophilus, Streptococcus agalactiae, Streptococcus parasanguinis, Streptococcus oralis, Streptococcus salivarius, Streptococcus macacae, Streptococcus dysgalactiae, Streptococcus anginosus, Streptococcus constellatus, Streptococcus pseudoporcinus, Streptococcus mutans, Listeria innocua, Spiroplasma apis, Spiroplasma syrphidicola, Porphyromonas catoniae, Prevotella intermedia, Treponema socranskii, Finegoldia magna, Pasteurella bettyae, Olivibacter sitiensis, Epilithonimonas tenax, Mesonia mobilis, Lactobacil lus plantarum, Coriobacteriaceae bacterium, Olsenella profusa, Haemophilus sputorum, Haemophilus pittmaniae, Pasteurella bettyae, Olivibacter sitiensis, Epilithonimonas tenax, M esonia mobilis, Lactobacillus plantarum, Bacillus cereus, Aquimarina muellen, Chryseobacte rium palustre, Bacteroides graminisolvens, Neisseria meningitidis, Francisella novicida, Flav obacteriumfrigidarium, Flavobacterium soli and/or Treponema denticola.

In certain embodiments, a Cas protein for use in the present disclosure directs cleavage of one or both strands at the location of a target nucleic acid. For example, but not by way of limitation, a Cas protein for use in the present disclosure directs cleavage of one or both strands within the target nucleic acid. Alternatively, the Cas protein directs cleavage of one or both strands within about 500 base pairs (e.g., within about 400, about 300, about 200, about 100, about 80, about 60, about 40, about 20, about 10 or about 5 base pairs) from the target nucleic acid.

In certain embodiments, a Cas protein, e.g., a Cas9, that includes functional RuvC and HNH nuclease domains can cleave both strands of a target nucleic acid sequence. In certain embodiments, the Cas protein, e.g., Cas9, comprises one functional endonuclease domain that allows the Cas protein to cleave only one strand (i.e., nick) of a target nucleic acid sequence. For example, but not by way of limitation, a Cas9 nickase can include (i) a non-functional RuvC domain (e.g., a mutant RuvC domain) and (ii) a functional HNH domain (e.g., a wild type HNH domain). In certain embodiments, a Cas9 nickase can comprise (i) a functional RuvC domain (e.g., wild type RuvC domain) and (ii) a non-functional HNH domain (e.g., a mutant HNH domain). In certain embodiments, a Cas9 nickase comprises a functional HNH-like and comprise a mutation at D10, e.g., D10A. In certain embodiments, a Cas9 nickase comprises a functional RuvC domain and comprises a mutation at H840, e.g., H840A. In certain embodiments, a Cas9 nickase comprises a functional RuvC domain and comprises a mutation at N863, e.g., N863A.

In certain embodiments, the nucleotide sequence encoding a Cas protein is codon optimized. For example, but not by way of limitation, the nucleotide sequence encoding a Cas protein can be codon optimized, e.g., where at least one non-common codon or less-common codon has been replaced by a common codon, for optimized expression in a particular cell type, e.g., a mammalian cell.

In certain embodiments, the Cas protein is a fusion protein that includes one or more heterologous protein domains. In certain embodiments, a Cas fusion protein can include any additional protein domains, e.g., epitope tags, reporter sequences and protein domains having one or more of the following activities: methylase activity, demethylase activity, transcription activation activity, transcription repression activity, transcription release factor activity, histone modification activity, RNA cleavage activity and nucleic acid binding activity.

In certain embodiments, the Cas protein includes one or more nuclear localization sequences to promote accumulation of the Cas protein in a detectable amount in the nucleus of a cell. Nuclear localization sequences are known in the art. For example, but not by way of limitation, a Cas protein can include a nuclear localization sequence (e.g., from SV40) at its N-terminus and/or C-terminus.

### IV. COMPOSITIONS

The present disclosure further provides compositions that include one or more modified gRNAs disclosed herein. In certain embodiments, a composition of the present disclosure can include two or more, three or more, four or more, five or more, six or more, seven or more, eight or more, nine or more or ten or more modified gRNAs disclosed herein.

In certain embodiments, a composition of the present disclosure includes a gRNA comprising one or more modifications disclosed herein, e.g., as described in Section II. In certain embodiments, the composition can include a nucleic acid that encodes a gRNA molecule disclosed herein. Alternatively or additionally, the composition includes the gRNA molecule as a transcribed or synthesized RNA molecule.

In certain embodiments, a composition of the present disclosure can further include an RNA-guided nuclease. In certain embodiments, the composition includes a nucleic acid that encodes an RNA-guided nuclease. Alternatively or additionally, the composition includes the RNA-guided nuclease as a protein. In certain embodiments, the RNA-guided nuclease is a Cas protein, e.g., a Cas9 protein.

The present disclosure further provides nucleic acid compositions that include one or more modified gRNAs disclosed herein. In certain embodiments, a nucleic acid composition includes a polynucleotide encoding a gRNA molecule described herein. In certain embodiments, the nucleic acid composition can further include a polynucleotide that encodes an RNA-guided nuclease, e.g., a Cas protein. In certain embodiments, a nucleic acid composition of the present disclosure can include a nucleic acid that encodes the gRNA molecule and the RNA-guided nuclease, e.g., a nucleic acid that includes a polynucleotide encoding a gRNA molecule (e.g., coupled to a first promoter) and a polynucleotide that encodes an RNA-guided nuclease (e.g., coupled to a second promoter). In certain embodiments, a nucleic acid composition of the present disclosure can include a first nucleic acid that encodes the gRNA molecule and a second nucleic acid that encodes the RNA-guided nuclease.

In certain embodiments, nucleic acid compositions encoding one or more gRNA molecules and/or one or more RNA-guided nucleases can be administered to subjects or delivered into cells by a method known in the art or as described herein. For example, but not by way of limitation, a nucleic acid encoding a gRNA molecule and/or an RNA-guided nuclease can be delivered to a subject or delivered into a cell by non-vector based methods (e.g., by using DNA complexes or naked DNA), by vector-based methods or a combination thereof. In certain embodiments, the vector can be a viral vector. In certain embodiments, the virus is an RNA virus or a DNA virus. Exemplary viral vectors/viruses include, but are not limited to, retroviruses, lentiviruses, adenovirus, adeno-associated virus (AAV), vaccinia viruses, poxviruses and herpes simplex viruses. In certain embodiments, a vector of the present disclosure includes a polynucleotide sequence that encodes a gRNA molecule and/or an RNA-guided nuclease. In certain embodiments, the vector further includes a sequence encoding a nuclear localization fused to the RNA-guided nuclease, e.g., at the N-terminus and/or C-terminus of the RNA-guided nuclease.

The present disclosure further provides compositions comprising Ribonucleoprotein (RNP) complexes. In certain embodiments, an RNP complex includes a gRNA molecule, e.g., as a transcribed or synthesized RNA, and an RNA-guided nuclease. In certain embodiments, the gRNA molecule forms a RNP complex with the RNA-guided nuclease under suitable condition prior to delivery to the target cells.

The present disclosure further provides cells that include one or more compositions of the present disclosure. For example, but not by way of limitation, a cell can include a nucleic acid composition of the present disclosure. In certain embodiments, a cell can include a RNP complex of the present disclosure. A variety of cells can be modified using the disclosed compositions. For example, but not by way of limitation, the cell can be an immune cell, e.g., a T cell. In certain embodiments, the T cell can be a CD8⁺ T cell and/or a CD4⁺ T cell. In certain embodiments, the cell can be a stem cell. In certain embodiments, the cell can be an induced pluripotent stem (iPS) cell. In certain embodiments, the cell can be a cell derived from a stem cell or an iPS, e.g., an immune cell derived from a stem cell or an iPS.

In certain embodiments, compositions of the present disclosure, e.g., nucleic acid compositions of the present disclosure, can be delivered into target cells by methods known in the art or as described herein. For example, but not by way of limitation, a composition of the present disclosure can be delivered into a cell by microinjection, electroporation, transient cell compression or squeezing, lipid-mediated transfection, peptide-mediated delivery or a combination thereof. In certain embodiments, a composition, e.g., comprising an RNP complex, is delivered to the target cells by electroporation.

### V. METHODS OF USE

The present disclosure further provides methods for using the gRNAs disclosed herein. In certain embodiments, gRNA molecules provided herein can be used for modifying a target nucleic acid in a cell. For example, but not by way of limitation, a gRNA molecule of the present disclosure can be used to modify a target nucleic acid in a cell, e.g., ex vivo or in vivo.

In certain embodiments, a gRNA molecule of the present disclosure can be used in a method to reduce the expression of the target nucleic acid in a cell. In certain embodiments, a gRNA molecule of the present disclosure can be used in a method to increase the expression of the target nucleic acid in a cell. In certain embodiments, a gRNA molecule of the present disclosure can be used in a method to introduce an insertion or deletion of one more nucleotides in close proximity to the target nucleic acid. In certain embodiments, a gRNA molecule of the present disclosure can be used in a method to introduce a deletion in close proximity to the target nucleic acid. In certain embodiments, a gRNA molecule of the present disclosure can be used in a method to introduce an insertion in close proximity to the target nucleic acid. In certain embodiments, a gRNA molecule of the present disclosure can be used in a method to introduce one or more breaks (e.g., double strand breaks or single strand breaks) in close proximity to the target nucleic acid.

In certain embodiments, a method for modifying a target nucleic acid in a cell includes contacting the cell with a gRNA molecule that includes a spacer (e.g., targeting domain) specific for a sequence in the target nucleic acid. In certain embodiments, the method includes contacting the cell with a composition disclosed herein, e.g., a composition comprising a modified gRNA molecule and an RNA-guided nuclease.

The present disclosure further provides methods for treating a subject. For example, but not by way of limitation, a composition comprising a gRNA molecule of the present disclosure can be used in a therapeutic method. In certain embodiments, the present disclosure provides a gRNA molecule for use in treating an individual in need thereof. In certain embodiments, the present disclosure provides a gRNA molecule for use in treating an individual having a disease. In certain embodiments, the gRNA molecule comprises a spacer that is complementary to a target nucleic acid, e.g., a gene that is associated with the disease to be treated. In certain embodiments, the method includes modifying a cell of the subject ex vivo by contacting the cell with a composition comprising an effective amount of a gRNA molecule disclosed herein or an RNP complex disclosed herein, e.g., by any of the delivery methods disclosed herein or known in the art. In certain embodiments, the method can further include returning the modified cell to the subject.

In certain embodiments, a gRNA molecule of the present disclosure can be used as a medicament. In certain embodiments, the present disclosure provides for the use of a gRNA molecule disclosed herein in the manufacture or preparation of a medicament. In certain embodiments, the medicament is for treatment of a disease. In certain embodiments, the medicament is for use in a method of treating a disease that includes administering to an individual having the disease an effective amount of the medicament.

In certain embodiments, compositions comprising any of the gRNA molecules provided herein, e.g., can be used in any of the above therapeutic methods. In certain embodiments, a composition comprising any of the gRNA molecules and/or RNA-guided nucleases provided herein can be used in a therapeutic method, e.g., modifying cells ex vivo.

In certain embodiments, one or more compositions disclosed herein, e.g., nucleic acid compositions, can be administered to a subject or contacted with a cell from a subject, e.g., ex vivo. In certain embodiments, the composition can include a gRNA of the present disclosure and an RNA-guided nuclease. Alternatively, the composition includes a gRNA of the present disclosure and does not include an RNA-guided nuclease. In certain embodiments, the composition, e.g., the nucleic acid composition, that includes the gRNA is administered at the same time as a composition that includes an RNA-guided nuclease. In certain embodiments, the composition, e.g., the nucleic acid composition, that includes the gRNA is administered after the composition that includes the RNA-guided nuclease. In certain embodiments, the composition, e.g., the nucleic acid composition, that includes the gRNA is administered prior to the composition that includes the RNA-guided nuclease. In certain embodiments, the nucleic acid composition can be delivered by any of the delivery methods described herein, e.g., a nucleic acid encoding the gRNA can be delivered by a viral vector. In certain embodiments, the composition that includes the RNA-guided nuclease can be delivered to a cell by electroporation.

### VI. GENE EDITING SYSTEMS

The present disclosure also provides gene editing systems comprising one or more compositions disclosed herein and/or materials useful for performing the methods described herein.

In certain embodiments, a gene editing system of the present disclosure can comprise one or more modified gRNA molecules disclosed herein. For example, but not by way of limitation, the gene editing system of the present disclosure can comprise one or more gRNA molecules disclosed in Section II.

In certain embodiments, the gRNA molecule of the gene editing system of the present disclosure comprises: (a) a phosphorodithioate linkage between the first and second nucleotides at the 5′ terminus of the guide RNA molecule; (b) a phosphorodithioate linkage between the 3′ terminal (“n”) nucleotide and the n−1 nucleotide of the guide RNA molecule; or (c) a phosphorodithioate linkage between the first and second nucleotides at the 5′ terminus of the guide RNA molecule and a phosphorodithioate linkage between the n and n−1 nucleotides of the guide RNA molecule. In certain embodiments, phosphorodithioate linkages are present between the first and second nucleotides, the second and third nucleotides, and the third and fourth nucleotides at the 5′ terminus of the gRNA molecule. In certain embodiments, phosphorodithioate linkages are present between the n and n−1 nucleotides and the n−1 and n−2 nucleotides or the n and n−1 nucleotides and the n−2 and n−3 nucleotides of the gRNA molecule.

In certain embodiments, the first, second, and/or third nucleotide at 5′ terminus of the gRNA molecule comprises a modification selected from the group consisting of: a 2′-fluoro modified nucleotide; a 2′-O-methyl modified nucleotide; a 2′-O-(2-methoxyethyl) modified nucleotide; a locked nucleic acid (LNA); a deoxyribose nucleotide; and a combination of two or more of the foregoing modifications. In certain embodiments, the first, second, third and/or fourth nucleotide at 5′ terminus of the gRNA molecule comprises a modification selected from the group consisting of: a 2′-fluoro modified nucleotide; a 2′-O-methyl modified nucleotide; a 2′-O-(2-methoxyethyl) modified nucleotide; a locked nucleic acid (LNA); a deoxyribose nucleotide; and a combination of two or more of the foregoing modifications. In certain embodiments, the first, second, third, fourth and/or fifth nucleotide at 5′ terminus of the gRNA molecule comprises a modification selected from the group consisting of: a 2′-fluoro modified nucleotide; a 2′-O-methyl modified nucleotide; a 2′-O-(2-methoxyethyl) modified nucleotide; a locked nucleic acid (LNA); a deoxyribose nucleotide; and a combination of two or more of the foregoing modifications.

In certain embodiments, the n, the n and n−1 nucleotides, the n and n−2, the n−1 and n−2 or the n, n−1 and n−2 nucleotides of the gRNA molecule comprise(s) a modification selected from the group consisting of: a 2′-fluoro modified nucleotide; a 2′-O-methyl modified nucleotide; a 2′-O-(2-methoxyethyl) modified nucleotide; a locked nucleic acid (LNA); a deoxyribose nucleotide; and a combination of two or more of the foregoing modifications. In certain embodiments, the n, n−1, n−2 and n−3 nucleotides or the n−1, n−2, n−3 and n−4 nucleotides of the gRNA molecule comprise(s) a modification selected from the group consisting of: a 2′-fluoro modified nucleotide; a 2′-O-methyl modified nucleotide; a 2′-O-(2-methoxyethyl) modified nucleotide; a locked nucleic acid (LNA); a deoxyribose nucleotide; and a combination of two or more of the foregoing modifications. In certain embodiments, the n, n−1, n−2, n−3 and n−4 nucleotides of the gRNA molecule comprise(s) a modification selected from the group consisting of: a 2′-fluoro modified nucleotide; a 2′-O-methyl modified nucleotide; a 2′-O-(2-methoxyethyl) modified nucleotide; a locked nucleic acid (LNA); a deoxyribose nucleotide; and a combination of two or more of the foregoing modifications.

In certain embodiments, the gene editing systems of the present disclosure can include one or more modified gRNAs, e.g., as disclosed in Section II, and an RNA-guided nuclease, e.g., an RNA-guided nuclease as disclosed in Section III. In certain embodiments, the RNA-guided nuclease is a Cas protein. In certain embodiments, the Cas protein is selected from the group consisting of a Cas9, a Cas12, a Cas13 and combinations thereof.

In certain embodiments, the gene editing systems of the present disclosure comprise one or more nucleic acids encoding a gRNA as described herein, e.g., as disclosed in Section II. In certain embodiments, the one or more nucleic acids further comprise a polynucleotide encoding an RNA-guided nuclease, e.g., an RNA-guided nuclease as disclosed in Section III. In certain embodiments, the RNA-guided nuclease is a Cas protein. In certain embodiments, the Cas protein is selected from the group consisting of a Cas9, a Cas12, a Cas13 and combinations thereof.

In certain embodiments, the gene editing system comprises one or more vectors comprising a nucleic acid of as described herein, e.g., as disclosed in Section IV.

In certain embodiments, the gene editing system comprises one or more compositions comprising a gRNA disclosed herein, e.g., as disclosed in Section II. In certain embodiments, the composition further comprises an RNA-guided nuclease. In certain embodiments, the composition comprises a nucleic acid described herein, e.g., as disclosed in Section IV.

In certain embodiments, the gene editing system comprises one or more RNP complexes comprising a gRNA of Section II and disclosed herein and an RNA-guided nuclease. In certain embodiments, the RNA-guided nuclease is a Cas protein. In certain embodiments, the Cas protein is selected from the group consisting of a Cas9, a Cas12, a Cas13 and combinations thereof.

### VII. EXEMPLARY EMBODIMENTS

A1. The presently disclosed subject matter provides a guide RNA molecule comprising:
   (a) a phosphorodithioate linkage between the first and second nucleotides at the 5′ terminus of the guide RNA molecule;
   (b) a phosphorodithioate linkage between the 3′ terminal (“n”) nucleotide and the n−1 nucleotide of the guide RNA molecule; or
   (c) a phosphorodithioate linkage between the first and second nucleotides at the 5′ terminus of the guide RNA molecule and a phosphorodithioate linkage between the n and n−1 nucleotides of the guide RNA molecule.
A2. The guide RNA molecule of A1 further comprising phosphorodithioate linkages between the second and third nucleotides and the third and fourth nucleotides at the 5′ terminus of the guide RNA molecule.
A3. The guide RNA molecule of A1 or A2 comprising phosphorodithioate linkages between the first and second nucleotides, the second and third nucleotides, and the third and fourth nucleotides at the 5′ terminus of the guide RNA molecule.
A4. The guide RNA molecule of any one of A1-A3 comprising phosphorodithioate linkages between the first and second nucleotides, the second and third nucleotides, the third and fourth nucleotides, and the fourth and fifth nucleotides at the 5′ terminus of the guide RNA molecule.
A5. The guide RNA molecule of any one of A1-A4 comprising phosphorodithioate linkages between the n and n−1 nucleotides and the n−1 and n−2 nucleotides or the n and n−1 nucleotides and the n−2 and n−3 nucleotides of the guide RNA molecule.
A6. The guide RNA molecule of any one of A1-A5 comprising phosphorodithioate linkages between the n and the n−1 nucleotides, the n−1 and n−2 nucleotides, and the n−2 and n−3 nucleotides of the guide RNA molecule.
A7. The guide RNA molecule of any one of A1-A6 comprising phosphorodithioate linkages between the n and n−1 nucleotides, the n−1 and n−2 nucleotides, the n−2 and n−3 nucleotides, and the n−3 and n−4 nucleotides of the guide RNA molecule.
A8. The guide RNA molecule of any one of A1-A7, wherein the first nucleotide at 5′ terminus of the guide RNA molecule comprises a modification selected from the group consisting of:
   (a) a 2′-fluoro modified nucleotide;
   (b) a 2′-O-methyl modified nucleotide;
   (c) a 2′-O-(2-methoxyethyl) modified nucleotide;
   (d) a locked nucleic acid (LNA);
   (e) a deoxyribose nucleotide; and
   (f) a combination of two or more of (a)-(e).
A9. The guide RNA molecule of A8, wherein the first nucleotide at 5′ terminus of the guide RNA molecule comprises a 2′-fluoro modified nucleotide.
A10. The guide RNA molecule of A8, wherein the first nucleotide at 5′ terminus of the guide RNA molecule comprises a 2′-O-methyl modified nucleotide.
A11. The guide RNA molecule of A8, wherein the first nucleotide at 5′ terminus of the guide RNA molecule comprises a 2′-O-(2-methoxyethyl) modified nucleotide.
A12. The guide RNA molecule of A8, wherein the first nucleotide at 5′ terminus of the guide RNA molecule comprises an LNA.
A13. The guide RNA molecule of A8, wherein the first nucleotide at 5′ terminus of the guide RNA molecule comprises a deoxyribose nucleotide.
A14. The guide RNA molecule of any one of A1-A13, wherein (i) the second nucleotide, (ii) the third nucleotide, (iii) the fourth nucleotide, (iv) the second and third nucleotides, (v) the second and fourth nucleotides, (vi) the third and fourth nucleotides or (v) the second, third and fourth nucleotides at the 5′ terminus of the guide RNA molecule comprises a modification selected from the group consisting of:
   (a) a 2′-fluoro modified nucleotide;
   (b) a 2′-O-methyl modified nucleotide;
   (c) a 2′-O-(2-methoxyethyl) modified nucleotide;
   (d) a locked nucleic acid (LNA);
   (e) a deoxyribose nucleotide; and
   (f) a combination of two or more of (a)-(e).
A15. The guide RNA molecule of any one of A1-A9 and A14, wherein the guide RNA molecule comprises three consecutive 2′-fluoro modified nucleotides at the first three nucleotides at the 5′ terminus of the guide RNA molecule.
A16. The guide RNA molecule of any one of A1-A8, A10, and A14, wherein the guide RNA molecule comprises three consecutive 2′-O-methyl modified nucleotides at the first three nucleotides at the 5′ terminus of the guide RNA molecule.
A17. The guide RNA molecule of any one of A1-A8, A10 and A14, wherein the guide RNA molecule comprises four consecutive 2′-O-methyl modified nucleotides at the first four nucleotides at the 5′ terminus of the guide RNA molecule.
A18. The guide RNA molecule of any one of A1-A8, A11 and A14, wherein the guide RNA molecule comprises three consecutive 2′-O-(2-methoxyethyl) modified nucleotides at the first three nucleotides at the 5′ terminus of the guide RNA molecule.
A19. The guide RNA molecule of any one of A1-A8, A12 and A14, wherein the guide RNA molecule comprises three consecutive LNAs at the first three nucleotides at the 5′ terminus of the guide RNA molecule.
A20. The guide RNA molecule of any one of A1-A8, A13 and A14, wherein the guide RNA molecule comprises three consecutive deoxyribose nucleotides at the first three nucleotides at the 5′ terminus of the guide RNA molecule.
A21. The guide RNA molecule of any one of A1-A20, wherein the 3′ terminal (“n”) nucleotide of the guide RNA molecule comprises a modification selected from the group consisting of:
   (a) a 2′-fluoro modified nucleotide;
   (b) a 2′-O-methyl modified nucleotide;
   (c) a 2′-O-(2-methoxyethyl) modified nucleotide;
   (d) a locked nucleic acid (LNA);
   (e) a deoxyribose nucleotide; and
   (f) a combination of two or more of (a)-(e).
A22. The guide RNA molecule of A21, wherein the n nucleotide of the guide RNA molecule comprises a 2′-fluoro modified nucleotide.
A23. The guide RNA molecule of A21, wherein the n nucleotide of the guide RNA molecule comprises a 2′-O-methyl modified nucleotide.
A24. The guide RNA molecule of A21, wherein the n nucleotide of the guide RNA molecule comprises a 2′-O-(2-methoxyethyl) modified nucleotide.
A25. The guide RNA molecule of A21, wherein the n nucleotide of the guide RNA molecule comprises an LNA.
A26. The guide RNA molecule of A21, wherein the n nucleotide of the guide RNA molecule comprises a deoxyribose nucleotide.
A27. The guide RNA molecule of any one of A1-A26, wherein (i) the n−1 nucleotide, (ii) the n−2 nucleotide, (iii) the n−3 nucleotide, (iv) the n−4 nucleotide, (v) the n and n−1 nucleotides, (vi) the n and n−2 nucleotides, (vii) the n−1 and n−2 nucleotides, (viii) the n, n−1 and n−2 nucleotides, (ix) the n−1, n−2 and n−3 nucleotides, (x) the n, n−1, n−2 and n−3 nucleotides, or (xi) the n, n−1, n−2, n−3 and n−4 nucleotides of the guide RNA molecule each comprise a modification selected from the group consisting of:
   (a) a 2′-fluoro modified nucleotide;
   (b) a 2′-O-methyl modified nucleotide;
   (c) a 2′-O-(2-methoxyethyl) modified nucleotide;
   (d) a locked nucleic acid (LNA);
   (e) a deoxyribose nucleotide; and
   (f) a combination of two or more of (a)-(e).
A28. The guide RNA molecule of any one A1-A22 and A27, wherein the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are 2′-fluoro modified nucleotides.
A29. The guide RNA molecule of any one of A1-A21, A23 and A27, wherein the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are 2′-O-methyl modified nucleotides.
A30. The guide RNA molecule of any one of A1-A21, A23, A27 and A29, wherein the n, n−1, n−2, n−3 and n−4 nucleotides, of the guide RNA molecule each are 2′-O-methyl modified nucleotides.
A31. The guide RNA molecule of any one of A1-A21, A24 and A27, wherein the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are 2′-O-(2-methoxyethyl) modified nucleotides.
A32. The guide RNA molecule of any one of A1-A21, A25 and A27, wherein the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are LNAs.
A33. The guide RNA molecule of any one of A1-A21, A26 and A27, wherein the n and n−1 nucleotides, the n and n−2 nucleotides, the n−1 and n−2 nucleotides, the n, n−1 and n−2 nucleotides, the n−1, n−2, n−3 and n−4 nucleotides or the n, n−1, n−2, n−3 and n−4 nucleotides of the guide RNA molecule each are deoxyribose nucleotides.
B. The presently disclosed subject matter provides a nucleic acid comprising a polynucleotide encoding a guide RNA molecule of any one of A1-A33.
B1. The nucleic acid of B further comprising a polynucleotide encoding an RNA-guided nuclease.
B2. The nucleic acid of B1, wherein the RNA-guided nuclease is a Cas protein.
B3. The nucleic acid of B2, wherein the Cas protein is selected from the group consisting of a Cas9, a Cas12, a Cas13 and a combination thereof.
C. The presently disclosed subject matter provides a vector comprising a nucleic acid of any one of B-B3.
D. The presently disclosed subject matter provides a composition comprising a guide RNA molecule of any one of A1-A33.
D1. The composition of D further comprising an RNA-guided nuclease.
D2. The composition of D or D1 further comprising a nucleic acid encoding the RNA-guided nuclease.
D3. The composition of D1 or D2, wherein the RNA-guided nuclease is a Cas protein.
E. The presently disclosed subject matter provides a composition comprising a nucleic acid of B-B3.
F. The presently disclosed subject matter provides a composition comprising a vector of C.
G. The presently disclosed subject matter provides a ribonucleoprotein (RNP) complex comprising a guide RNA molecule of any one of A1-A33 and an RNA-guided nuclease.
G1. The RNP complex of G, wherein the RNA-guided nuclease is a Cas protein.
G2. The RNP complex of G1, wherein the Cas protein is selected from the group consisting of a Cas9, a Cas12, a Cas13 and a combination thereof.
H. The presently disclosed subject matter provides a cell comprising a composition of any one of D-D3, E, or F.
I. The presently disclosed subject matter provides a cell comprising a RNP complex of any one of G-G2.
J. The presently disclosed subject matter provides a method of modifying a cell comprising contacting the cell with a composition of any one of D-D3, E, or F, or an RNP complex of any one of G-G2.
J1. The method of J, wherein said contacting comprises introducing the composition into the cell by electroporation.
K. The presently disclosed subject matter provides a method of treating a subject in need thereof, comprising:
   (a) modifying a cell of the subject ex vivo by contacting the cell with a composition of any one of D-D3, E, or F, or an RNP complex of any one of G-G2; and
   (b) returning the modified cell to the subject.
L. The presently disclosed subject matter provides a gene editing system comprising:
   (a) one or more guide RNA molecules of any one of A1-A33;
   (b) one or more nucleic acids of any one of B-B3;
   (c) one or more vectors of C;
   (d) one or more composition of D-D3, E, or F; and/or
   (e) one or more RNP complexes of any one of G-G2.

### EXAMPLES

The presently disclosed subject matter will be better understood by reference to the following Example, which is provided as exemplary of the presently disclosed subject matter, and not by way of limitation.

### Example 1: Modifications of gRNA Molecules

This Example provides gRNA molecules that were chemically synthesized to include multiple modifications at the 5′ and 3′ termini of the gRNA molecules. As shown in Table 1, this Example provides modification strategies A-L. Table 1 provides a summary of the modifications for each of these strategies compared to the reference gRNA molecule.

Nucleotide modifications are indicated in Table 1 as follows: *: phosphorothioate (PS) linkage; **: phosphorodithioate (PS2) linkage; m: 2′-OMe; In: locked nucleic acid (LNA); f: 2′-fluoro; M: 2′-O-MOE; and d: deoxyribonucleotide. For example, mA represents 2′-O-methyl adenosine and dA represents an adenosine deoxyribonucleotide. “N” recited in the sequences of Table 1 represents any nucleotide base, e.g., guanine (G), adenine (A), thymine (T) or cytosine (C). The reference gRNAs disclosed in Table 1, which are referred to as Reference gRNA 1, Reference gRNA 2 and Reference gRNA 3, have the same nucleotide sequence as the modified gRNAs of strategies A-L. Reference gRNAs 1 and 3 has the same modification strategy as shown in Table 1. Reference gRNA 2 has 3 phosphorothioate linkages at the 3′ end and terminates with rU compared to Reference gRNAs 1 and 3.

TABLE 1

| | Modification | | |
|---|---|---|---|
| Name of | Strategy at 5′ | Modification Strategy at | |
| gRNA | Terminus | 3′ Terminus | Sequence |
| Reference | 2′-O-methyl | 2′-O-methyl | 5′-mN*mN*mN*rNrNrNrNrNrNrNrNrNrN |
| gRNA 1 | modifications for | modifications for | rNrNrNrNrNrNrNrGrUrUrUrUrArGrArGrCrUr |
| | first 3 consecutive | terminal 3 | ArGrArArArUrArGrCrArArGrUrUrArArArAr |
| | nucleotides | nucleotides | UrArArGrGrCrUrArGrUrCrCrGrUrUrArUrCr |
| | 3 | 2 phosphorothioate | ArArCrUrUrGrArArArArArGrUrGrGrCrArCr |
| | phosphorothioate | linkages | CrGrArGrUrCrGrGrUrGrCrUmU*mU*mU-3′ |
| | linkages | | (SEQ ID NO: 1) |
| Reference | 2′-O-methyl | 2′-O-methyl | 5′-mN*mN*mN*rNrNrNrNrNrNrNrNrNrN |
| gRNA 2 | modifications for | modifications for n-1 | rNrNrNrNrNrNrNrGrUrUrUrUrArGrArGrCrUr |
| | first 3 consecutive | and n-2 nucleotides | ArGrArArArUrArGrCrArArGrUrUrArArArAr |
| | nucleotides | 3 phosphorothioate | UrArArGrGrCrUrArGrUrCrCrGrUrUrArUrCr |
| | 3 | linkages | ArArCrUrUrGrArArArArArGrUrGrGrCrArCr |
| | phosphorothioate | | CrGrArGrUrCrGrGrUrGrCrU*mU*mU*rU-3′ |
| | linkages | | (SEQ ID NO: 2) |
| Reference | 2′-O-methyl | 2′-O-methyl | 5′-mN*mN*mN*rNrNrNrNrNrNrNrNrNrN |
| gRNA 3 | modifications for | modifications for | rNrNrNrNrNrNrNrGrUrUrUrUrArGrArGrCrUr |
| | first 3 consecutive | terminal 3 | ArGrArArArUrArGrCrArArGrUrUrArArArAr |
| | nucleotides | nucleotides | UrArArGrGrCrUrArGrUrCrCrGrUrUrArUrCr |
| | 3 | 2 phosphorothioate | ArArCrUrUrGrArArArArArGrUrGrGrCrArCr |
| | phosphorothioate | linkages | CrGrArGrUrCrGrGrUrGrCrUmU*mU*mU-3′ |
| | linkages | | (SEQ ID NO: 3) |
| A | Replace 2′-O- | Replace 2′-O-methyl | 5′-InN*InN*InN*rNrNrNrNrNrNrNrNrN |
| | methyl modified | modified nucleotides | rNrNrNrNrNrNrNrNrGrUrUrUrUrArGrArGrCr |
| | nucleotides with | with LNAs | UrArGrArArArUrArGrCrArArGrUrUrArArAr |
| | LNAs | | ArUrArArGrGrCrUrArGrUrCrCrGrUrUrArUr |
| | | | CrArArCrUrUrGrArArArArArGrUrGrGrCrAr |
| | | | CrCrGrArGrUrCrGrGrUrGrCrUlnU*InU*InU-3′ |
| | | | (SEQ ID NO: 4) |
| B | Replace | Replace | 5′-mN**mN**mN**rNrNrNrNrNrNrNrNr |
| | phosphorothioate | phosphorothioate | NrNrNrNrNrNrNrNrNrGrUrUrUrUrArGrArGr |
| | linkages with | linkages with | CrUrArGrArArArUrArGrCrArArGrUrUrArAr |
| | phosphorodithioate | phosphorodithioate | ArArUrArArGrGrCrUrArGrUrCrCrGrUrUrAr |
| | linkages | linkages | UrCrArArCrUrUrGrArArArArArGrUrGrGrCr |
| | | | ArCrCrGrArGrUrCrGrGrUrGrCrUmU**mU** |
| | | | mU-3′ (SEQ ID NO: 5) |
| C | Replace 2′-O- | Replace 2′-O-methyl | 5′-MN*MN*MN*rNrNrNrNrNrNrNrNrNr |
| | methyl modified | modified nucleotides | NrNrNrNrNrNrNrNrGrUrUrUrUrArGrArGrCr |
| | nucleotides with | with 2′-O-(2- | UrArGrArArArUrArGrCrArArGrUrUrArArAr |
| | 2′-O-(2- | methoxyethyl)- | ArUrArArGrGrCrUrArGrUrCrCrGrUrUrArUr |
| | methoxyethyl)- | modified nucleotides | CrArArCrUrUrGrArArArArArGrUrGrGrCrAr |
| | modified | | CrCrGrArGrUrCrGrGrUrGrCrUMU*MU*MU-3′ |
| | nucleotides | | (SEQ ID NO: 6) |
| D | Replace 2′-O- | Replace 2′-O-methyl | 5′-fN*fN*fN*rNrNrNrNrNrNrNrNrNrNrNr |
| | methyl modified | modified nucleotides | NrNrNrNrNrNrGrUrUrUrUrArGrArGrCrUrAr |
| | nucleotides with | with 2′-fluoro- | GrArArArUrArGrCrArArGrUrUrArArArArUr |
| | 2′-fluoro-modified | modified nucleotides | ArArGrGrCrUrArGrUrCrCrGrUrUrArUrCrAr |
| | nucleotides | | ArCrUrUrGrArArArArArGrUrGrGrCrArCrCr |
| | | | GrArGrUrCrGrGrUrGrCrUfU*fU*fU-3′ (SEQ |
| | | | ID NO: 7) |
| E | No changes to | No changes to | 5′-mN*mN*mN*fNfNfNfNfNfNfNfNfNfN |
| | Reference gRNA | Reference gRNA 1 | fNfNfNfNfNfNfNfGfUfUfUfUfAfGfAfGfCfUf |
| | 1 | | AfGfAfAfAfUfAfGfCfAfAfGfUfUfAfAfAfAf |
| | | | UfAfAfGfGfCfUfAfGfUfCfCfGfUfUfAfUfCf |
| | | | AfAfCfUfUfGfAfAfAfAfAfGfUfGfGfCfAfCf |
| | | | CfGfAfGfUfCfGfGfUfGfCfUmU*mU*mU-3′ |
| | | | (SEQ ID NO: 8) |
| F | Replace 2′-O- | Replace 2′-O-methyl | 5′-dN**dN**dN**rNrNrNrNrNrNrNrNrNr |
| | methyl modified | modified nucleotides | NrNrNrNrNrNrNrNrGrUrUrUrUrArGrArGrCr |
| | nucleotides with | with deoxyribose | UrArGrArArArUrArGrCrArArGrUrUrArArAr |
| | deoxyribose | nucleotides | ArUrArArGrGrCrUrArGrUrCrCrGrUrUrArUr |
| | nucleotides | Replace | CrArArCrUrUrGrArArArArArGrUrGrGrCrAr |
| | Replace | phosphorothioate | CrCrGrArGrUrCrGrGrUrGrCrUdU**dU**dU-3′ |
| | phosphorothioate | linkages with | (SEQ ID NO: 9) |
| | linkages with | phosphorodithioate | |
| | phosphorodithioate | linkages | |
| | linkages | | |
| G | Remove | Remove | 5′-mNmNmNrNrNrNrNrNrNrNrNrNrNr |
| | phosphorothioate | phosphorothioate | NrNrNrNrNrNrNrGrUrUrUrUrArGrArGrCrUr |
| | linkages | linkages | ArGrArArArUrArGrCrArArGrUrUrArArArAr |
| | | | UrArArGrGrCrUrArGrUrCrCrGrUrUrArUrCr |
| | | | ArArCrUr UrGrArArArArArGrUrGrGrCrArCr |
| | | | CrGrArGrUrCrGrGrUrGrCrUmUmU mU-3′ |
| | | | (SEQ ID NO: 10) |
| H | No changes to | No changes to | 5′-mN*mN*mN*rNrNrNrNrNrNrNrNrNr |
| | Reference gRNA | Reference gRNA 1 | NrNrNrNrNrNrNrNrGrU*rU*rU*rU*rA*rG*r |
| | 1 | | ArGrCrUrArGrArArArUrArGrCrArArG*rU*r |
| | | | U*rArA*rArA*rUrA*rArGrGrCrUrArG*rU*rC |
| | | | rCrG*rU*rU*rA*rUrCrA*rA*rCrUrUrGrArAr |
| | | | ArArArGrUrGrG*rCrArCrCrGrArGrUrCrGrGr |
| | | | UrGrCrUmU*mU*mU-3′ (SEQ ID NO: 11) |
| I | No changes to | No changes to | 5′-mN*mN*mN*dNdNdNdNdNdNdNdN |
| | Reference gRNA | Reference gRNA 1 | dNdNdNdNdNdNdNdNdNrGrUrUrUrUrArGrA |
| | 1 | | rGrCrUrArGrArArArUrArGrCrArArGrUrUrAr |
| | | | ArArArUrArArGrGrCrUrArGrUrCrCrGrUrUr |
| | | | ArUrCrArArCrUrUrGrArArArArArGrUrGrGr |
| | | | CrArCrCrGrArGrUrCrGrGrUrGrCrUmU*mU* |
| | | | mU-3′ (SEQ ID NO: 12) |
| J | Remove 2′-O- | Remove 2′-O-methyl | 5′-rN*rN*rN*rNrNrNrNrNrNrNrNrNrNrN |
| | methyl | modifications | rNrNrNrNrNrNrGrUrUrUrUrArGrArGrCrUrAr |
| | modifications | | GrArAr ArUrArGrCrArArGrUrUrArArArArUr |
| | | | ArArGrGrCrUrArGrUrCrCrGrUrUrArUrCrAr |
| | | | ArCrUrUrGrArArArArArGrUrGrGrCrArCrCr |
| | | | GrArGrUrCrGrGrUrGrCrUrU*rU*rU-3′ (SEQ |
| | | | ID NO: 13) |
| K | Replace 2′-O- | No changes to | 5′-dN**dN**dN**rNrNrNrNrNrNrNrNrN |
| | methyl modified | Reference gRNA 1 | rNrNrNrNrNrNrNrNrGrUrUrUrUrArGrArGrCr |
| | nucleotides with | | Ur ArGr ArAr ArUrArGrCrArArGrUrUrArArAr |
| | deoxyribose | | ArUrArArGrGrCrUrArGrUrCrCrGrUrUrArUr |
| | nucleotides | | CrArArCrUrUrGrArArArArArGrUrGrGrCrAr |
| | 3 | | CrCrGrArGrUrCrGrGrUrGrCrUmU*mU*mU-3′ |
| | phosphorodithioate | | (SEQ ID NO: 14) |
| | linkages | | |
| L | Remove all | No changes to | 5′-rNrNrNrNrNrNrNrNrNrNrNrNrNrNrN |
| | modifications | Reference gRNA 1 | rNrNrNrNrNrGrUrUrUrUrArGrArGrCrUrArGr |
| | | | ArArArUrArGrCrArArGrUrUrArArArArUrAr |
| | | | ArGrGrCrUrArGrUrCrCrGrUrUrArUrCrArArC |
| | | | rUrUrGrArArArArArGrUrGrGrCrArCrCrGrAr |
| | | | GrUrCrGrGrUrGrCrUmU*mU*mU-3′ (SEQ ID |
| | | | NO: 15) |

The identity of the synthesized modified gRNAs was confirmed by mass spectrometry with less than 200 ppm mass error between experimental and theoretical masses. As shown in Tables 2 and 3, the experimental masses were consistent with the calculated ones confirming correct length and nucleobase composition.

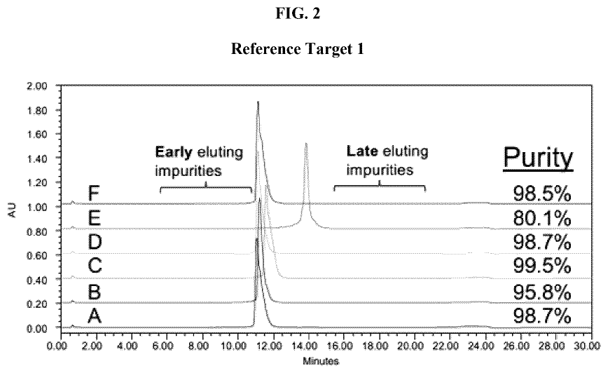

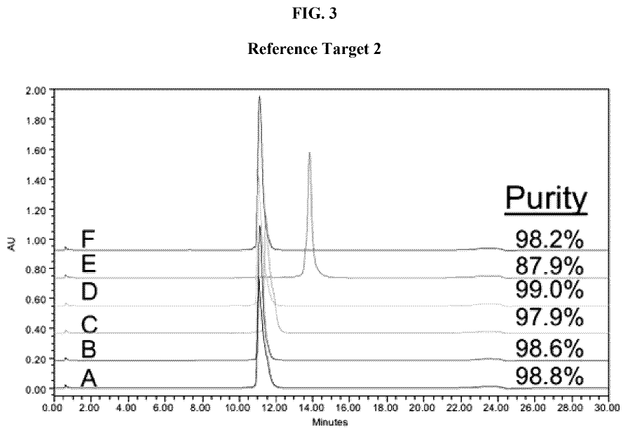

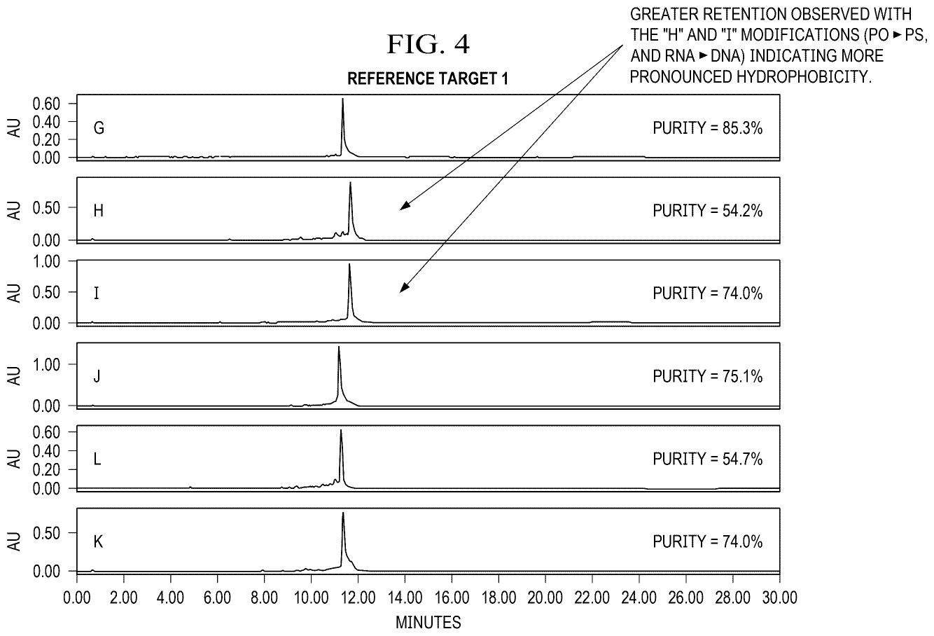

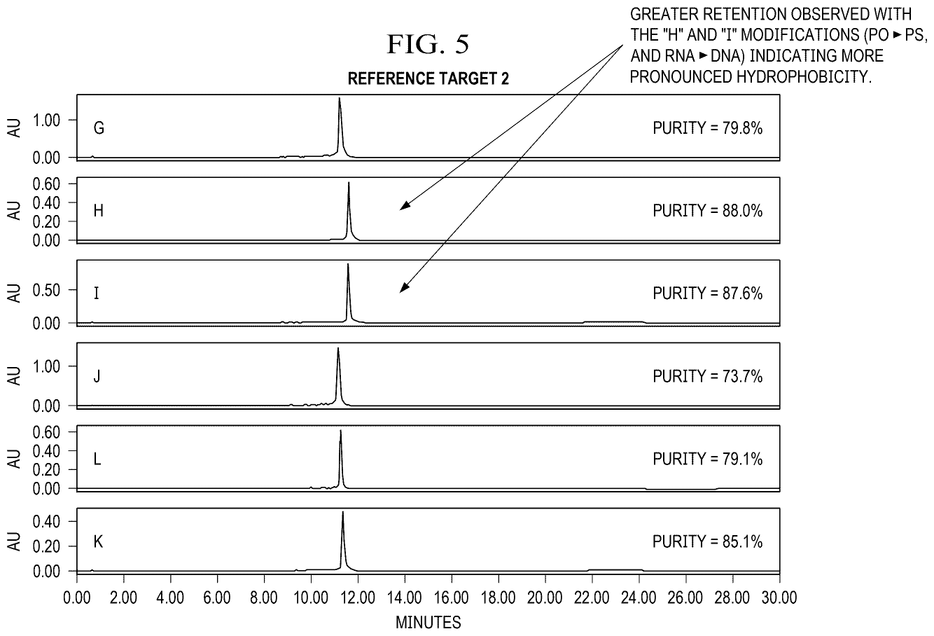

The purity of the synthesized modified gRNAs was analyzed. As shown in FIGS. 2 and 3, similar purity profiles were observed for gRNAs with modifications according to modification schemes A-F. Purity of these modified gRNAs ranged between 80.1% and 99.5%. FIGS. 2 and 4 and Table 2 provide the purity profiles of a gRNA targeting Reference Target 1 and modified according to modification schemes A-L, and FIGS. 3 and 5 and Table 3 provide the purity profiles of a gRNA targeting Reference Target 2 and modified according to modification schemes A-L. As shown in FIGS. 2 and 3, the sequence of the gRNA has a negligible effect on the purity and ion-pairing reversed-phase liquid chromatography (IPRP) profiles on the modified gRNAs having modifications according to modification schemes A-F. Similar results were observed for gRNAs targeting these two target sequences and having modifications according to modification schemes G-L (FIGS. 4-5 and Tables 2 and 3). In addition, impurities were observed to be primarily shortmers.

As noted in FIG. 4, the 2′-fluoro modification significantly increased sgRNA hydrophobicity. Without being bound to a particular theory, greater hydrophobicity can enable binding of an oligonucleotide to interact with certain domains of a protein and modulate its activity (see Crooke et al., Nucleic Acids Research 48(10):5235-5253 (2020), the contents of which are incorporated herein by reference in their entirety).

TABLE 2

| Modification | | | | |
|---|---|---|---|---|
| Strategy of gRNA | | | | |
| Targeting | | Theoretical | Experimental | Difference |
| Reference | Purity | Mass | average Mass | in Mass |
| Target 1 | (% a/a) | (Da) | (Da) | (Da) |
| A | 98.7 | 32428.7 | 32426.8 | −1.9 |
| B | 95.8 | 32465.0 | 32464.0 | −1.0 |
| C | 99.5 | 32705.1 | 32705.0 | −0.1 |
| D | 98.7 | 32312.5 | 32312.5 | 0.0 |
| E | 80.1 | 32571.9 | 32572.0 | 0.1 |
| F | 98.5 | 32327.0 | 32326.4 | −0.6 |
| G | 85.3 | 32304.3 | 32304.5 | 0.2 |
| H | 54.2 | 32722.0 | 32720.4 | −1.6 |
| I | 74.0 | 32112.7 | 32112.0 | −0.7 |
| J | 75.1 | 32300.5 | 32300.5 | 0.0 |
| K | 74.0 | 32342.8 | 32342.0 | −0.8 |
| L | 54.7 | 32294.4 | 32294.0 | −0.4 |

TABLE 3

| Modification | | | | |
|---|---|---|---|---|
| Strategy of gRNA | | | | |
| Targeting | | Theoretical | Experimental | Difference |
| Reference | Purity | Mass | average Mass | in Mass |
| Target 2 | (% a/a) | (Da) | (Da) | (Da) |
| A | 98.8 | 32379.7 | 32374.3 | −5.4 |
| B | 98.6 | 32416.0 | 32415.5 | −0.5 |
| C | 97.9 | 32656.1 | 32656.4 | 0.3 |
| D | 99.0 | 32263.5 | 32263.0 | −0.5 |
| E | 87.9 | 32522.8 | 32523.0 | 0.2 |
| F | 98.2 | 32292.0 | 32291.5 | −0.5 |
| G | 79.8 | 32255.3 | 32255.0 | −0.3 |
| H | 88.0 | 32673.0 | 32673.0 | 0.0 |
| I | 87.6 | 32063.6 | 32063.6 | 0.0 |
| J | 73.7 | 32251.5 | 32251.6 | 0.1 |
| K | 85.1 | 32307.8 | 32307.6 | −0.2 |
| L | 79.1 | 32245.4 | 32245.5 | 0.1 |

The efficacy of the above-described gRNA molecules targeting Reference Target 1 and modified as shown in Table 1 were analyzed as follows. Cells were plated in Prime-XV (IL-7 (25 ng/ml), IL-15 (50 ng/ml), TransAct 1:100) (=Day 0). 4 mg of each sgRNA were resuspended in 611-624 μL (depending on MW) of IDT Duplex buffer (Catalog #1072570) to obtain a 200 μM solution. RNPs were generated using a 3:1 sgRNA:Cas9 ratio. For knock-out conditions, 1.11, 3.33, 10 and 30 pmols RNP were titrated. For knock-in conditions, 30 pmols RNP were used with the addition of 2 μg of a Reference Target 1-mNeon nanoplasmid. The components were added in the following order: 1—RNP (sgRNA and Cas9 preincubated 15 min at RT first), 2—Template (3 j g) and 3—Cells (2 million). Cells were electroporated at 46 hours post-stimulation in P3 buffer on Lonza 4D electroporator. Post-electroporation, cells were left at 37° C. for 15 min. 75 μl of plain Prime-XV was added and the cells were transferred to 1 ml of complete media without TransAct in 48-well plates. 3 ml of complete media was added on day 5 followed by the transfer of the cells to a 12-well plate. 0.5 mL of the culture was removed for FACs readout on day 5 (KO samples only). 0.5 mL of culture the culture was removed for FACs readout on day 6 (KI samples only). The cells were subsequently stained at days 5 and 6 post-stimulation to detect the reduction or increase in the expression of Reference Target 1.

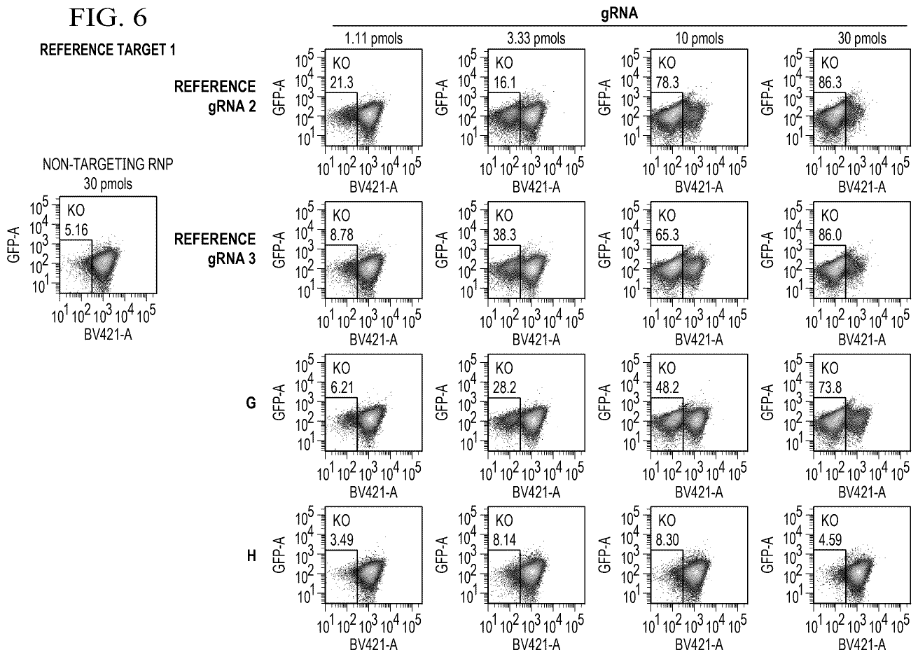

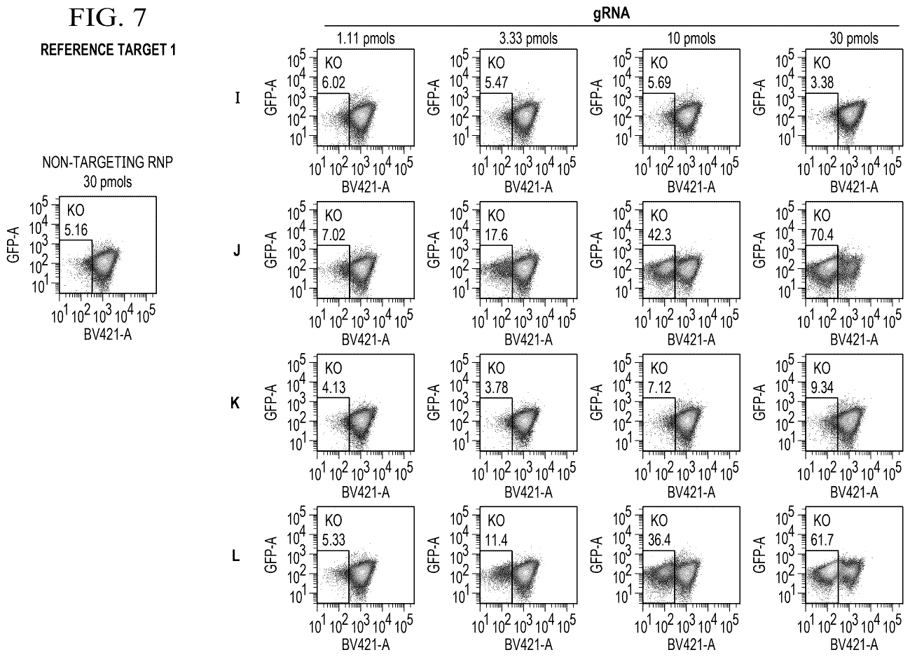

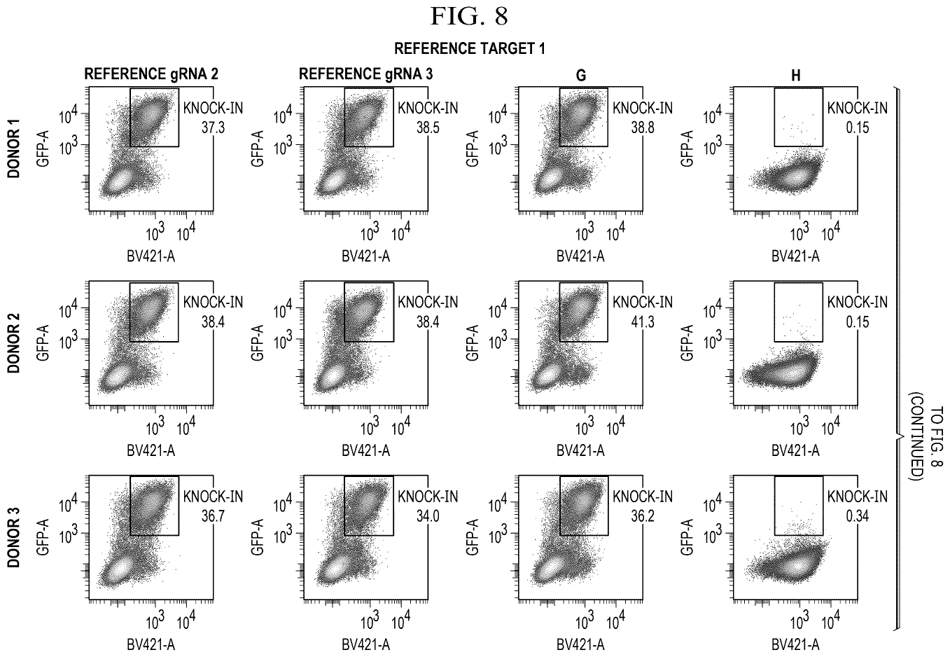

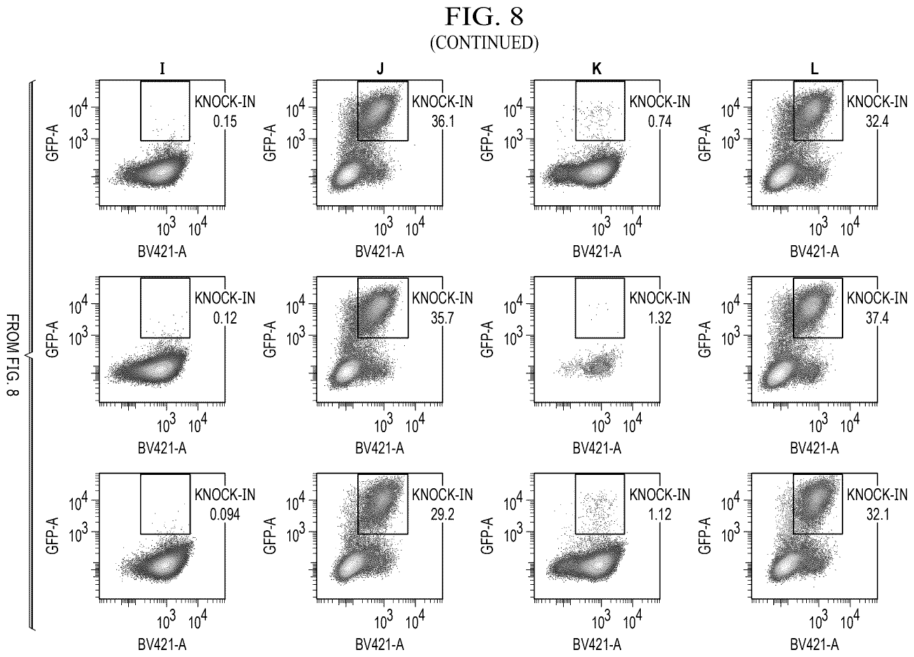

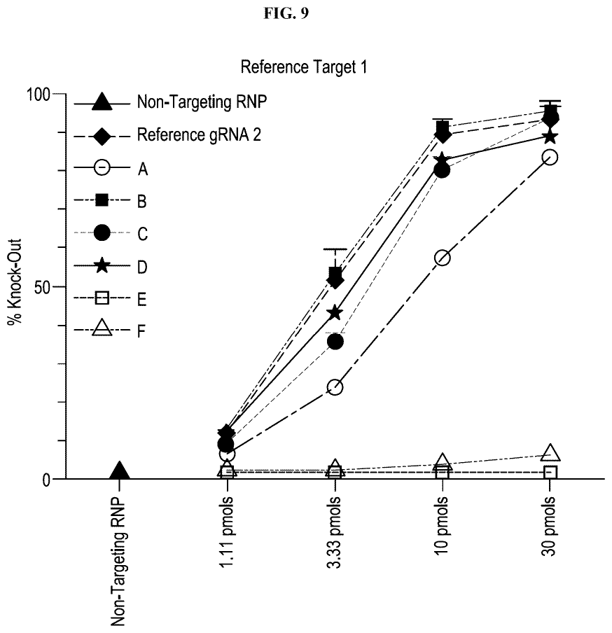

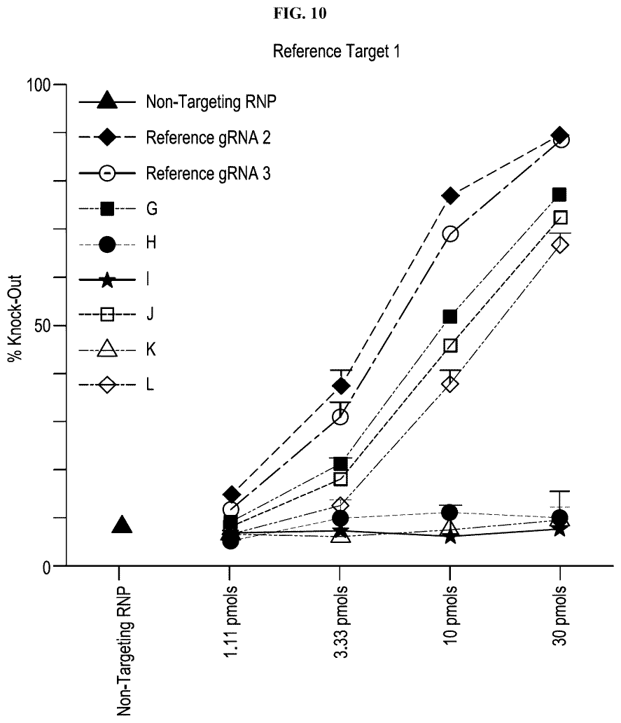

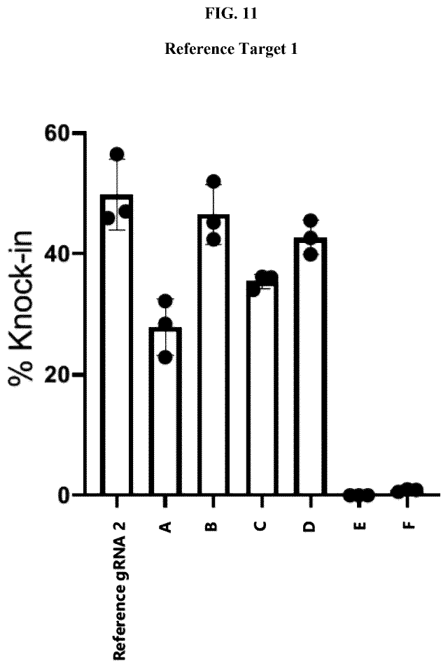

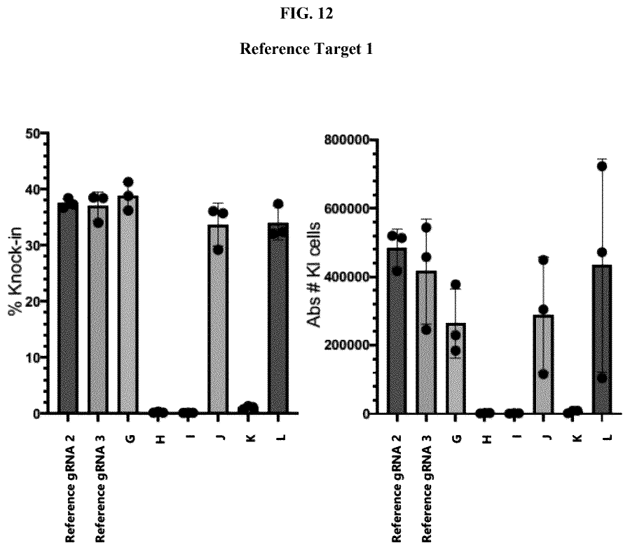

The editing efficiencies of the modified gRNAs shown in FIGS. 6-12 are summarized in Tables 4 and 5. As shown in FIGS. 9 and 11 and Table 4, the “B” gRNA performed very similarly to the reference gRNA under both knock-out and knock-in conditions. gRNA “D” outperformed gRNA “C” and gRNA “C” outperformed gRNA “A”. gRNAs “E” and “F” showed very little activity compared to reference gRNAs under both knock-out and knock-in conditions.

As shown in FIGS. 6-8, 10 and 12 and Table 5, gRNAs “G,” “J” and “L” exhibited a moderate level of knock-out activity compared to the reference gRNAs but showed similar knock-in activity as the two reference gRNAs. gRNAs “H” and “I” resulted in no knock-out or knock-in activity. In addition, gRNA “K” produced a very small amount of knock-out or knock-in activity compared to the reference gRNAs.

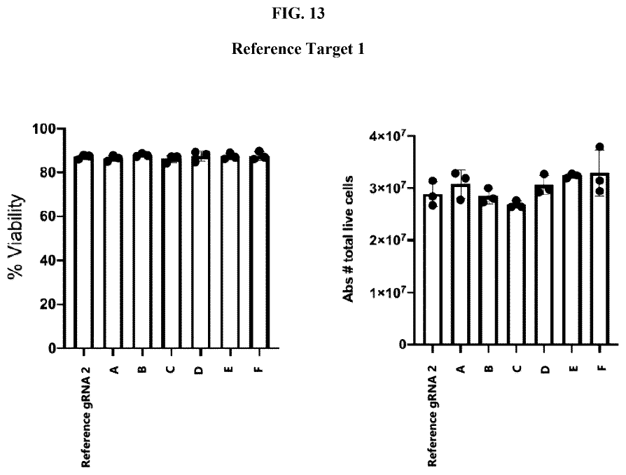

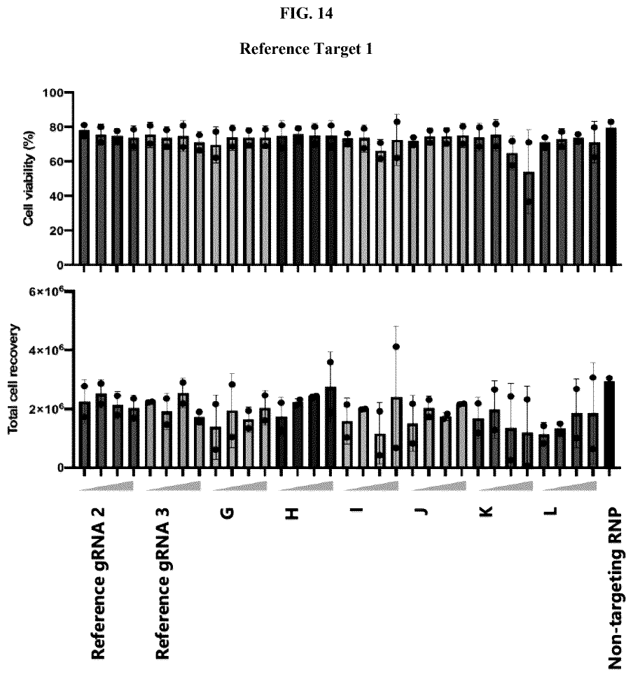

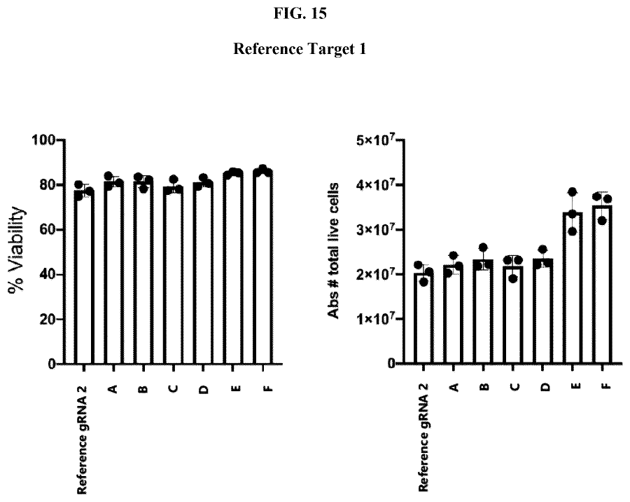

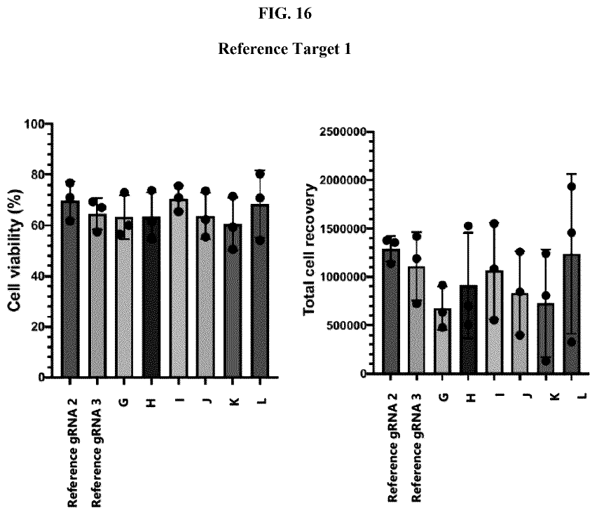

The viability of the cells under both knock-out and knock-in conditions in the presence of the modified gRNAs were tested. As shown in FIGS. 13-16, the viability of the cells was very similar for all the modified gRNAs tested under knock-out (FIGS. 13-14) and knock-in (FIGS. 15-16) conditions. Similarly, the total number of cells was very similar for most of the gRNAs tested under knock-out (FIGS. 13-14) and knock-in (FIGS. 15-16) conditions. However, for the knock-in experiment, more total cells were measured for gRNA “E” and gRNA “F”, which can be due to the failure of these gRNAs to efficiently cut the target nucleic acid (FIG. 15).

TABLE 4

| | Mean KO activity | Mean KI activity |
|---|---|---|
| Name of gRNA | (10 pmols) | (30 pmols) |
| Reference gRNA 2 | 89% | 50% |
| A | 58% | 29% |
| B | 91% | 47% |
| C | 80% | 35% |
| D | 83% | 43% |
| E | 2% | 0% |
| F | 4% | 1% |

TABLE 5

| | Mean KO activity | Mean KI activity |
|---|---|---|
| Name of gRNA | (10 pmols) | (30 pmols) |
| Reference gRNA 2 | 77% | 37% |
| Reference gRNA 3 | 70% | 37% |
| G | 52% | 39% |
| H | 11% | 0% |
| I | 6% | 0% |
| J | 46% | 34% |
| K | 8% | 1% |
| L | 38% | 34% |

As summarized in Tables 4 and 5, gRNAs “B,” “C” and “D” exhibited the greatest editing efficacy compared to the other gRNAs tested and were more comparable to the control.

### Example 2: Forced Degradation of Modified gRNA Molecules

This example shows the degradation of the exemplary gRNA molecules described in Example 1 under forced conditions. The gRNA molecules of Example 1 were subjected to forced degradation conditions including acidic stress (pH 5), basic stress (pH 11), oxidative stress and thermal stress conditions.

### Forced Degradation Conditions:

Acidic Stress (pH 5): Acidic buffer: 20 mM of sodium acetate, pH 5.0. Temperature: 40° C. Time Points: 0, 1, 2, and 3 days (gRNAs “A” to “L”); 0, 1, 2, 3, 5, and 7 days (gRNAs “4M” to “V”).

Basic Stress (pH 11): Basic buffer: 20 mM of sodium carbonate pH 11.0. Temperature: 40° C. Time Points: 0, 8, 16, and 24 hours (gRNAs “A” to “L”); 0, 8, 16, 24, and 48 hours (gRNAs “M” to “V”).

Oxidative Stress: 0.3% H₂O₂. Temperature: 40° C. Time Points: 0, 1, 2, 3 and 5 days (gRNAs “A” to “V”).

Thermal Stress: Tris-EDTA (TE) Buffer, pH 8.0. Temperature: 40° C. Time Points: 0, 1, 3, 5 and 7 days (gRNAs “A” to “V”).

### Sample Preparation Methods:

Acidic Stress (pH 5) Analysis: To prepare the acidic stress (pH 5) solution, 164 mg of sodium acetate was added into a 100 mL volumetric flask. 80 mL of LC grade water was added to the flask and the pH was adjusted to 5.0 with acetic acid. The solution was volumed up with water. 10 μL of sgRNA was pipetted into a HPLC insert and 55 μL of acidic stress (pH 5) solution was added followed by gentle vortexing for 5 s.

Basic Stress (pH 11) Analysis: To prepare the basic stress (pH 11) solution, 212 mg of sodium carbonate was weighed and placed into a 100 mL volumetric flask. About 80 mL of LC grade water was added to the flask to dissolve the sodium carbonate and the pH was adjusted to 11.0 with 1N HCl. The solution was volumed up with water. 10 μL of sgRNA was pipetted into a HPLC insert and 55 μL of basic stress (pH 11) solution was added followed by gentle vortexing for 5 s.

Thermal (liquid) Analysis: 10 μL of sgRNA was pipetted into a HPLC insert and 55 μL of a TE buffer (pH 8) was added followed by gentle vortexing for 5 s.

Oxidation Analysis: To prepare the oxidative solution, 0.59 mL of 3% H₂O₂ was added into a 5 mL volumetric flask and volumed up TE buffer followed by mixing. 10 μL of sgRNA was pipetted into a HPLC insert and 55 μL of 0.35% H₂O₂ was added followed by gentle vortexing for 5 s.

HPLC: HPLC was performed as shown in Table 6.

TABLE 6

| Parameters | Condition | |
|---|---|---|
| Sampler temperature | 40° C. | |
| Column | DNAPac RP, 4 μm, 2.1 × 100 mm | |
| Flow rate | 0.40 mL/min | |
| Column Temperature | 70° C. | |
| Mobile Phase | A: 400 mM HFIP + 16.3 mM TEA in water | |
| | B: 40% MPA + 60% Methanol | |
| Injection volume | 2 μL | |
| UV Wavelength | 260 nm | |
| Gradient-(min) | MPA: | MPB: |
| 0 | 90 | 10 |
| 20 | 40 | 60 |
| 21 | 0 | 100 |
| 23 | 0 | 100 |
| 23.5 | 90 | 10 |
| 30 | 90 | 10 |

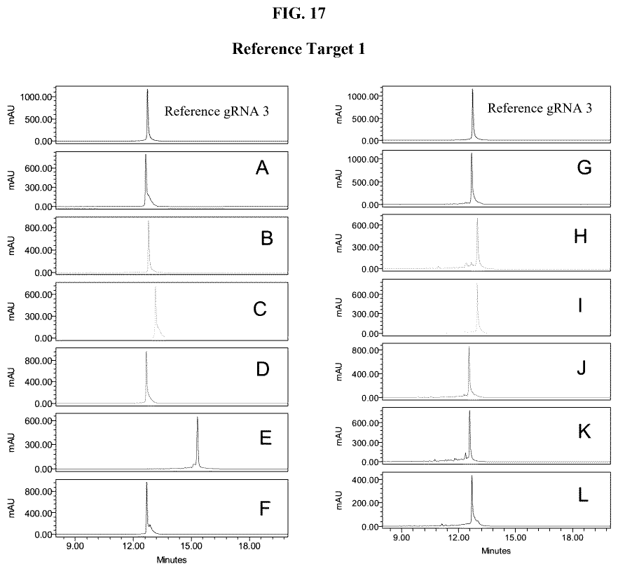

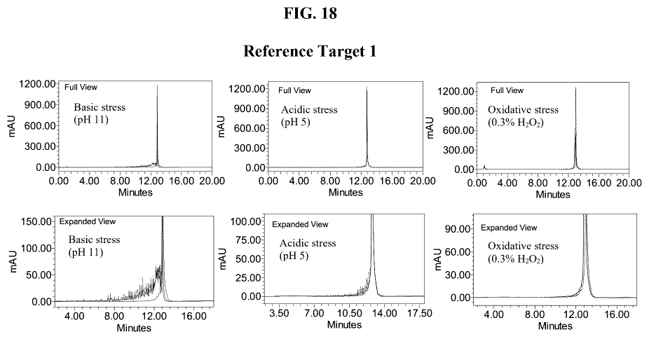

FIG. 17 shows the elution profiles of the modified gRNAs under normal conditions. As shown in FIG. 17, the gRNAs with the modification strategies A-L had an initial purity ranging from 47%-92% at Timepoint 0 (T₀). The results of the forced degradation studies of these modified gRNAs are shown in Tables 7-9 and FIGS. 18-40.

TABLE 7
Purity Summary under Forced Degradation

| | Purity (area %) | | | | |
|---|---|---|---|---|---|
| Name of | | 0.3% H₂O₂, | pH 8, 40° | pH 5, 40° | pH 11, 40° |
| gRNA | T(0) | 40° C., T(5 d) | C., T(7 d) | C., T(3 d) | C., T(1 d) |
| Reference | 91.4 | 91.2 | 86.0 | 81.9 | 11.2 |
| gRNA 3 | | | | | |
| A | 66.6 | 63.7 | 62.8 | 61.0 | 11.8 |
| B | 89.3 | 78.3 | 82.2 | 81.6 | 23.0 |
| C | 64.6 | 22.4 | 61.0 | 59.9 | 16.6 |
| D | 93.4 | 85.0 | 88.9 | 85.1 | 75.9 |
| E | 67.7 | 61.0 | — | 66.5 | 67.0 |
| F | 63.5 | 61.3 | 60.6 | 59.8 | 52.6 |
| G | 65.3 | 59.4 | 59.5 | 60.2 | 0.8 |
| H | 52.3 | 3.9 | 47.7 | 49.6 | 19.9 |
| I | 66.9 | 64.4 | 63.8 | 64.2 | 9.3 |
| J | 56.3 | 55.2 | 52.3 | 52.9 | 38.2 |
| K | 47.4 | 45.8 | 42.6 | 44.5 | 11.7 |
| L | 47.8 | 47.7 | 45.5 | 45.5 | 7.0 |

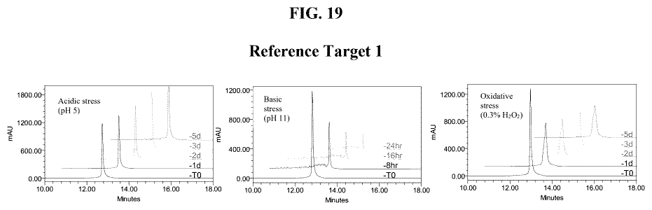

FIG. 18 shows the chromatograms of reference gRNA 3 under basic stress (pH 11), acidic stress (pH 5) and oxidative stress (0.3% H₂O₂). FIG. 19 shows an overlap of the chromatograms of the reference gRNA 3 under basic stress (pH 11), acidic stress (pH 5) and oxidative stress (0.3% H₂O₂) over certain time periods. As shown in FIGS. 18-19, reference gRNA 3 was more resistant to acid hydrolysis (e.g., resists degradation on exposure to acid) in comparison to basic stress (pH 11) which led to the formation of shortmers from rapid degradation.

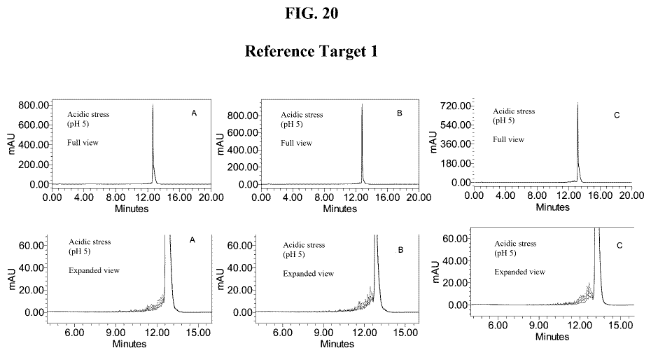

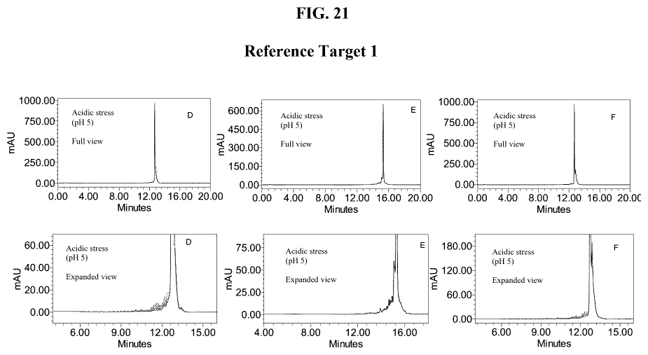

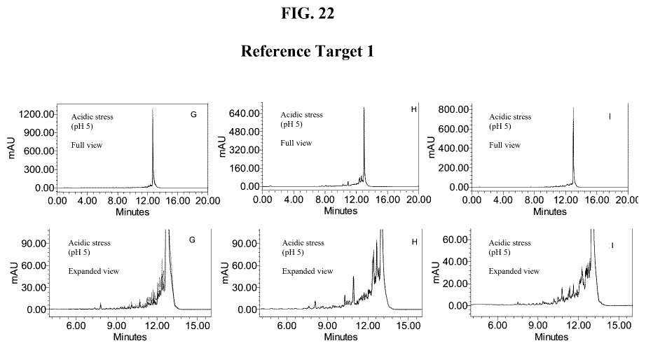

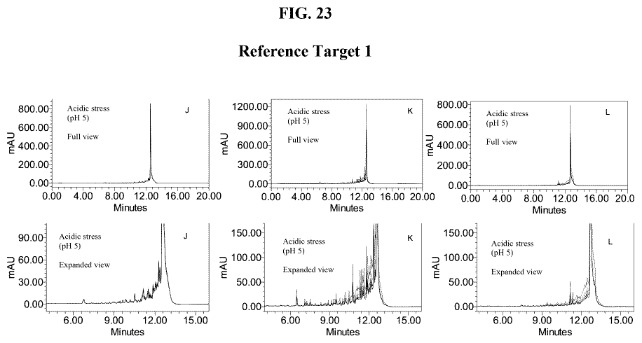

FIG. 20 shows the chromatograms of the gRNAs A, B and C under acidic stress (pH 5). FIG. 21 shows the chromatograms of modified gRNAs D, E and F under acidic stress (pH 5). FIG. 22 shows the chromatograms of modified gRNAs G, H and I under acidic stress (pH 5). FIG. 23 shows the chromatograms of the gRNAs J, K and L under acidic stress (pH 5). As shown in FIGS. 20-23, gRNAs E, F, H, I, and J were more resistant to acidic stress.

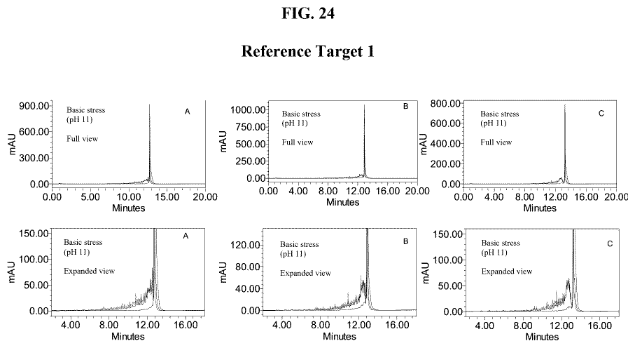

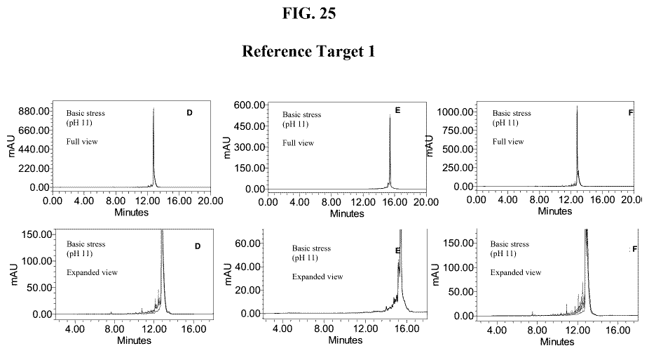

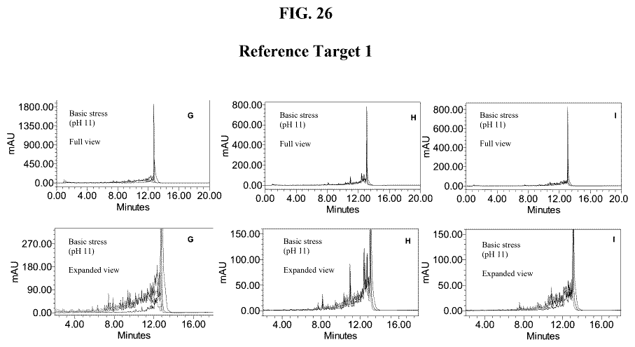

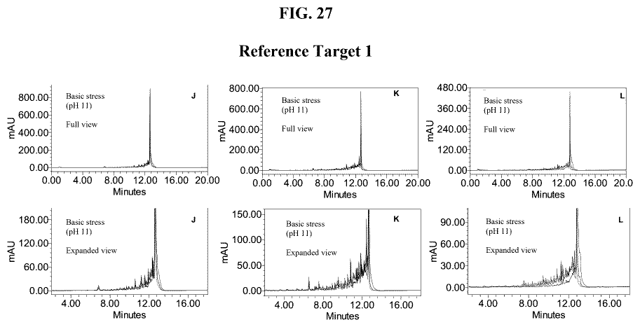

FIG. 24 shows the chromatograms of the gRNAs A, B and C under basic stress (pH 11). FIG. 25 shows the chromatograms of modified gRNAs D, E and F under basic stress (pH 11). FIG. 26 shows the chromatograms of modified gRNAs G, H and I under basic stress (pH 11). FIG. 27 shows the chromatograms of modified gRNAs J, K and L under basic stress (pH 11). As shown in FIGS. 24-27, modified gRNAs D, E, F, H, I, J, and K were resistant to basic stress (pH 11).

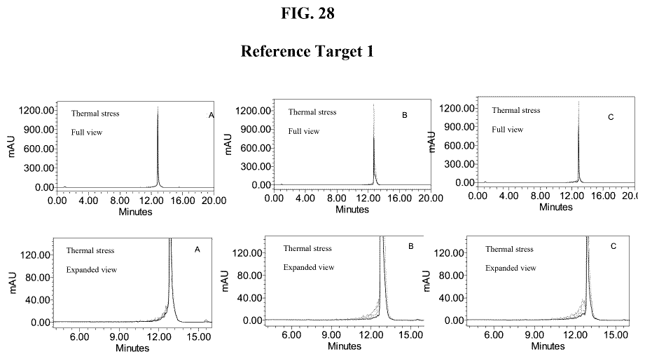

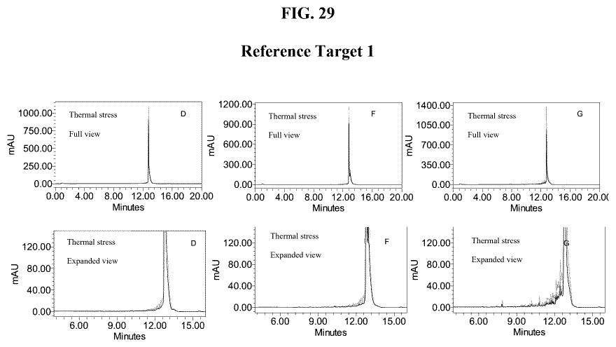

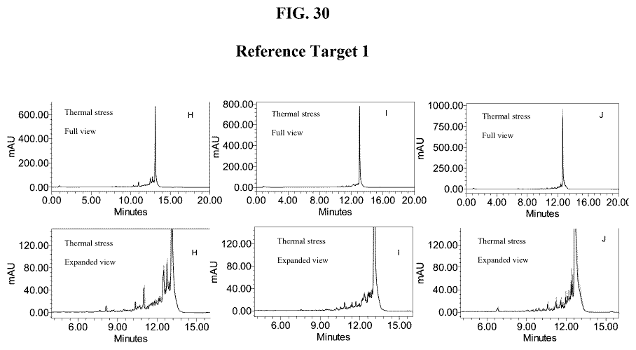

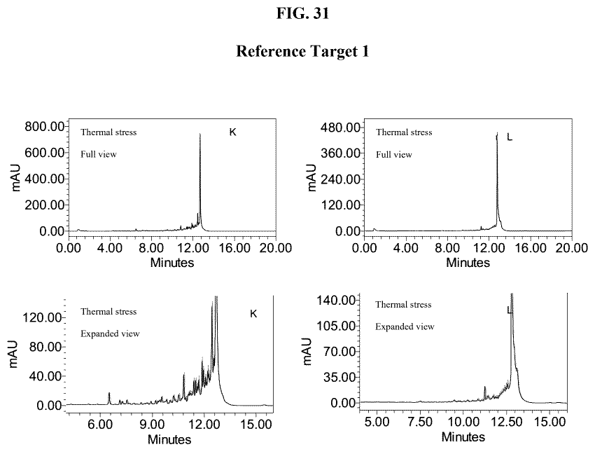

FIG. 28 shows the chromatograms of the gRNAs A, B and C under thermal stress. FIG. 29 shows the chromatograms of modified gRNAs D, F and G under thermal stress. FIG. 30 shows the chromatograms of modified gRNAs H, I and J under thermal stress. FIG. 31 shows the chromatograms of modified gRNAs K and L under thermal stress. As shown in FIGS. 28-31, most gRNAs were resistant to thermal degradation.

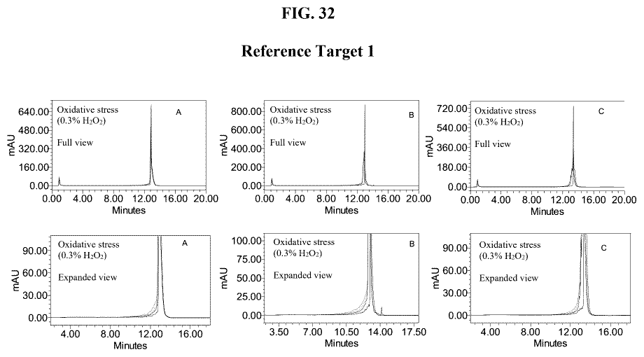

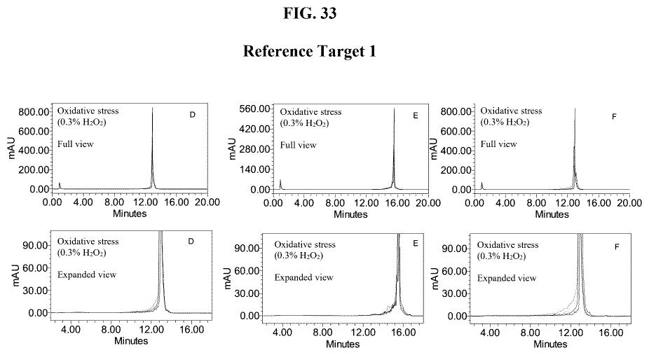

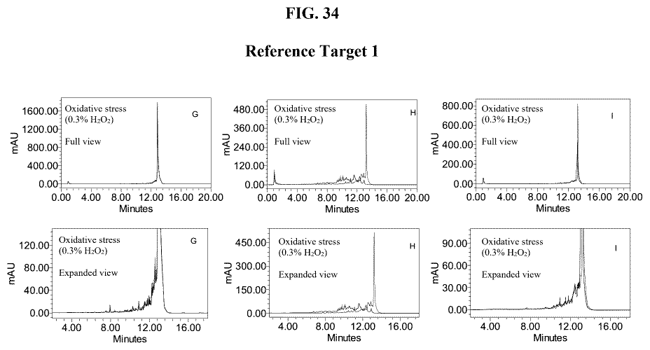

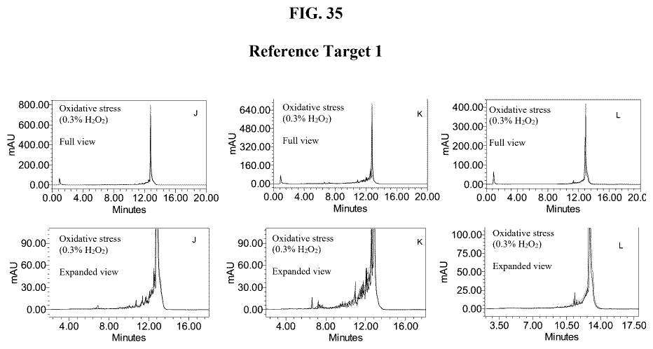

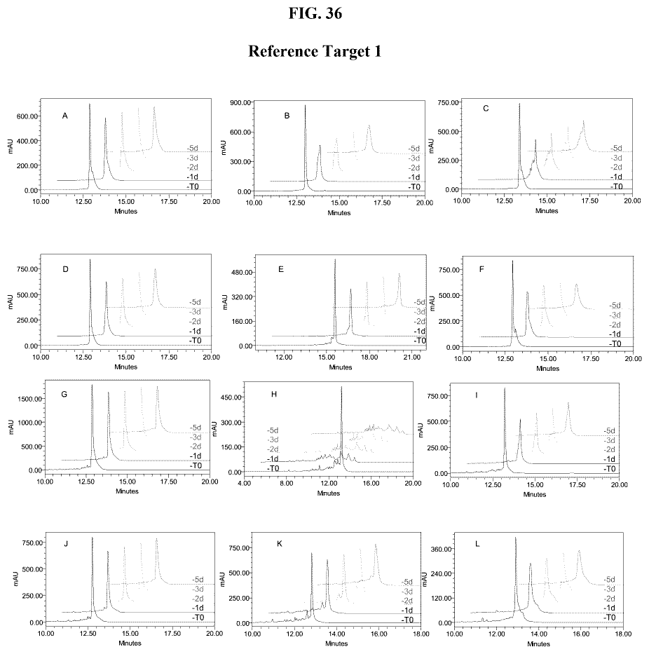

FIG. 32 shows the chromatograms of modified gRNAs A, B and C under oxidative stress (0.3% H₂O₂). FIG. 33 shows the chromatograms of modified gRNAs D, E and F under oxidative stress (0.3% H₂O₂). FIG. 34 shows the chromatograms of modified gRNAs G, H and I under oxidative stress (0.3% H₂O₂). FIG. 35 shows the chromatograms of modified gRNAs J, K and L under oxidative stress (0.3% H₂O₂). FIG. 36 shows an overlap of the chromatograms of modified gRNAs A-L under oxidative stress (0.3% H₂O₂) over a 5-day period. As shown in FIGS. 32-36, gRNAs E, G, I, J, K, and L were more resistant to oxidative stress except for gRNAs C and H.

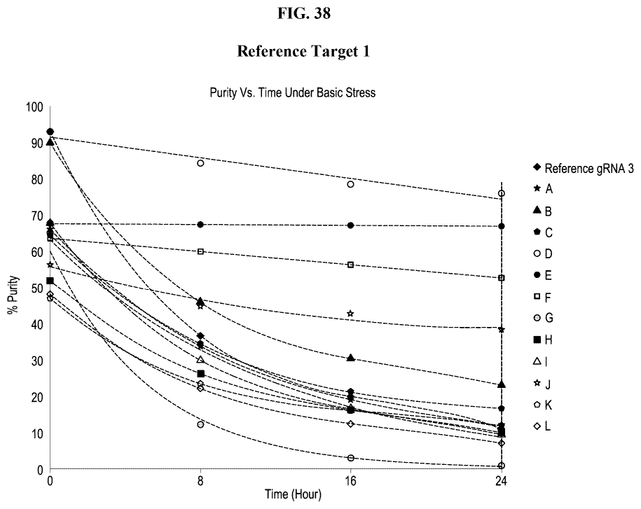

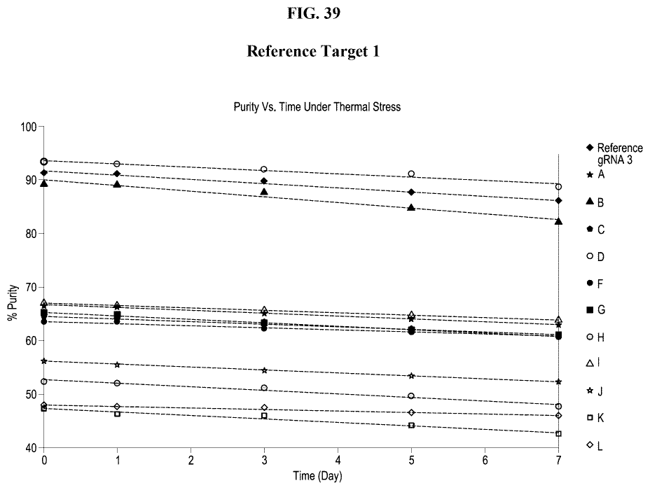

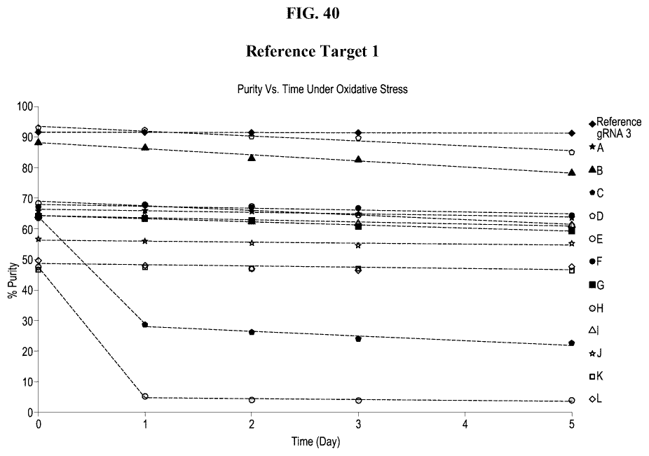

FIG. 37 shows the changes in purity over a 3-day time period during acidic stress (pH 5). FIG. 38 shows the changes in purity over a 24-hour period during basic stress (pH 11). FIG. 39 shows the changes in purity over a 7-day time period during thermal stress. As shown in FIG. 39, there was not enough of gRNA E available for the thermal stress study. FIG. 40 shows the changes in purity over a 5-day time period under oxidative stress (0.3% H₂O₂).

As shown in Table 7, many of the modified gRNAs resisted degradation under forced degradation conditions. For example, gRNAs “D,” “E,” “F” and “J” resisted degradation conditions including under basic stress (pH 11) conditions compared to the reference gRNA (Table 7).

Tables 8 and 9 provide a summary of the changes in purity for the modified gRNAs under acid hydrolysis (Table 8) and under thermal stress (Table 9). The slope shows the decrease in purity per day, the intercept shows the number of days for the purity decreases to 0 and R is the correlation coefficient for linearity curve.

TABLE 8

| Name of gRNA | Linear Function | Correction Coefficient R |
|---|---|---|
| Reference gRNA 3 | y = −3.165x + 91.6 | 0.998 |
| A | y = −1.692x + 65.588 | 0.967 |
| B | y = −2.708x + 89.262 | 0.986 |
| C | y = −1.376x + 64.214 | 0.962 |
| D | y = −2.702x + 93.073 | 0.998 |
| E | y = −0.125x + 66.970 | 0.965 |
| F | y = −1.416x + 63.684 | 0.970 |
| G | y = −1.828x + 64.972 | 0.951 |
| H | y = −0.65x + 51.575 | 0.990 |
| I | y = −0.533x + 66.437 | 0.986 |
| J | y = −0.450x + 55.455 | 0.966 |
| K | y = −0.754x + 46.876 | 0.964 |
| L | y = −1.335x + 49.32 | 0.986 |

TABLE 9

| Name of gRNA | Linear Function | Correction Coefficient R |
|---|---|---|
| Reference gRNA 3 | y = −0.7998x + 91.763 | 0.990 |
| A | y = −0.5379x + 66.753 | 0.991 |
| B | y = −1.0456x + 89.982 | 0.979 |
| C | y = −0.4806x + 64.432 | 0.990 |
| D | y = −0.6165x + 93.653 | 0.978 |
| F | y = −0.4057x + 63.624 | 0.987 |
| G | y = −0.6451x + 65.244 | 0.988 |
| H | y = −0.6665x + 52.715 | 0.981 |
| I | y = −0.4537x + 67.004 | 0.998 |
| J | y = −0.5459x + 56.121 | 0.996 |
| K | y = −0.6445x + 47.372 | 0.980 |
| L | y = −0.2826x + 47.982 | 0.963 |

### Example 3: Modifications of gRNA Molecules

This example provides additional gRNA molecules that were chemically synthesized to include multiple modifications at the 5′ and 3′ termini of the gRNA molecules. As shown in Table 10, this example provides modification strategies B and M-V. Modification strategy B provided in Table 10 is the same strategy as modification strategy B shown in Example 1 and Table 1. Table 10 provides a summary of the modifications for each of these strategies, and Table 11 provides a comparison of the modifications for each of these strategies to Reference gRNA 3.

Nucleotide modifications are indicated in Table 10 as follows: *: phosphorothioate (PS) linkage; **: phosphorodithioate (PS2) linkage; m: 2′-OMe; In: locked nucleic acid (LNA); f: 2′-fluoro; M: 2′-O-MOE; and d: deoxyribonucleotide. “N” recited in the sequences of Table 10 represents any nucleotide base, e.g., guanine (G), adenine (A), thymine (T) or cytosine (C).

TABLE 10

| Name of | Modification Strategy at | Modification Strategy at 3′ | |
|---|---|---|---|
| gRNA | 5′ Terminus | Terminus | Sequence |
| B | 2′-O-methyl | 2′-O-methyl modifications | 5′- |
| | modifications for first | for terminal 3 nucleotides | mN**mN**mN**rNrNrNrNrNrNrNrN |
| | 3 consecutive | 2 phosphorodithioate | rNrNrNrNrNrNrNrNrNrGrUrUrUrUrAr |
| | nucleotides | linkages (between n and n- | GrArGrCrUrArGrArArArUrArGrCrAr |
| | 3 phosphorodithioate | 1 and between n-1 and n-2) | ArGrUrUrArArArArUrArArGrGrCrUr |
| | linkages | | ArGrUrCrCrGrUrUrArUrCrArArCrUr |
| | | | UrGrArArArArArGrUrGrGrCrArCrCr |
| | | | GrArGrUrCrGrGrUrGrCrUmU**mU** |
| | | | mU-3′ (SEQ ID NO: 16) |
| M | 2′-O-methyl | 3 deoxyribose nucleotides | 5′- |
| | modifications for first | for terminal 3 nucleotides | mN**mN**mN**rNrNrNrNrNrNrNrN |
| | 3 consecutive | 2 phosphorodithioate | rNrNrNrNrNrNrNrNrNrGrUrUrUrUrAr |
| | nucleotides | linkages (between n and n- | GrArGrCrUrArGrArArArUrArGrCrAr |
| | 3 phosphorodithioate | 1 and between n-1 and n-2) | ArGrUrUrArArArArUrArArGrGrCrUr |
| | linkages | | ArGrUrCrCrGrUrUrArUrCrArArCrUr |
| | | | UrGrArArArArArGrUrGrGrCrArCrCr |
| | | | GrArGrUrCrGrGrUrGrCrUdU**dU**d |
| | | | U-3′ (SEQ ID NO: 17) |
| N | Deoxyribose | 2′-O-methyl modifications | 5′- |
| | nucleotides for first 3 | for terminal 3 nucleotides | dN**dN**dN**rNrNrNrNrNrNrNrNrN |
| | consecutive | 2 phosphorothioate | rNrNrNrNrNrNrNrNrGrUrUrUrUrArGr |
| | nucleotides | linkages (between n and n- | ArGrCrUrArGrArArArUrArGrCrArAr |
| | 3 phosphorodithioate | 1 and between n-1 and n-2) | GrUrUrArArArArUrArArGrGrCrUrAr |
| | linkages | | GrUrCrCrGrUrUrArUrCrArArCrUrUr |
| | | | GrArArArArArGrUrGrGrCrArCrCrGr |
| | | | ArGrUrCrGrGrUrGrCrUmU*mU*mU- |
| | | | 3′ (SEQ ID NO: 18) |
| O | 2′-O-methyl | 3 deoxyribose nucleotides | 5′- |
| | modifications for first | for terminal 3 nucleotides | mN*mN*mN*rNrNrNrNrNrNrNrNrNr |
| | 3 consecutive | 2 phosphorodithioate | NrNrNrNrNrNrNrNrGrUrUrUrUrArGr |
| | nucleotides | linkages (between n and n- | ArGrCrUrArGrArArArUrArGrCrArAr |
| | 3 phosphorothioate | 1 and between n-1 and n-2) | GrUrUrArArArArUrArArGrGrCrUrAr |
| | linkages | | GrUrCrCrGrUrUrArUrCrArArCrUrUr |
| | | | GrArArArArArGrUrGrGrCrArCrCrGr |
| | | | ArGrUrCrGrGrUrGrCrUdU**dU**dU- |
| | | | 3′ (SEQ ID NO: 19) |
| P | 2′-O-methyl | 2′-O-methyl modifications | 5′- |
| | modifications for first | for terminal 3 nucleotides | mN**mN**mN**rNrNrNrNrNrNrNrN |
| | 3 consecutive | 2 phosphorothioate | rNrNrNrNrNrNrNrNrNrGrUrUrUrUrAr |
| | nucleotides | linkages (between n and n- | GrArGrCrUrArGrArArArUrArGrCrAr |
| | 3 phosphorodithioate | 1 and between n-1 and n-2 | ArGrUrUrArArArArUrArArGrGrCrUr |
| | linkages | | ArGrUrCrCrGrUrUrArUrCrArArCrUr |
| | | | UrGrArArArArArGrUrGrGrCrArCrCr |
| | | | GrArGrUrCrGrGrUrGrCrUmU*mU*m |
| | | | U-3′ (SEQ ID NO: 20) |
| Q | 2′-O-methyl | 2′-O-methyl modifications | 5′- |
| | modifications for first | for terminal 3 nucleotides | mN*mN*mN*rNrNrNrNrNrNrNrNrNr |
| | 3 consecutive | 2 phosphorodithioate | NrNrNrNrNrNrNrNrGrUrUrUrUrArGr |
| | nucleotides | linkages (between n and n- | ArGrCrUrArGrArArArUrArGrCrArAr |
| | 3 phosphorothioate | 1 and between n-1 and n-2) | GrUrUrArArArArUrArArGrGrCrUrAr |
| | linkages | | GrUrCrCrGrUrUrArUrCrArArCrUrUr |
| | | | GrArArArArArGrUrGrGrCrArCrCrGr |
| | | | ArGrUrCrGrGrUrGrCrUmU**mU**m |
| | | | U-3′ (SEQ ID NO: 21) |
| R | 2′-O-methyl | 2′-O-methyl modifications | 5′- |
| | modifications for first | for terminal 3 nucleotides | mN*mN**mN**rNrNrNrNrNrNrNrNr |
| | 3 consecutive | 2 phosphorodithioate | NrNrNrNrNrNrNrNrNrGrUrUrUrUrAr |
| | nucleotides | linkages (between n and n- | GrArGrCrUrArGrArArArUrArGrCrAr |
| | 1 phosphorothioate | 1 and between n-1 and n-2) | ArGrUrUrArArArArUrArArGrGrCrUr |
| | linkage between first | | ArGrUrCrCrGrUrUrArUrCrArArCrUr |
| | and second | | UrGrArArArArArGrUrGrGrCrArCrCr |
| | nucleotides | | GrArGrUrCrGrGrUrGrCrUmU**mU** |
| | 2 phosphorodithioate | | mU-3′ (SEQ ID NO: 22) |
| | linkages (between | | |
| | second and third and | | |
| | third and fourth) | | |
| S | 2′-O-methyl | 2′-O-methyl modifications | 5′- |
| | modifications for first | for terminal 3 nucleotides | mN**mN**mN*rNrNrNrNrNrNrNrNr |
| | 3 consecutive | 2 phosphorodithioate | NrNrNrNrNrNrNrNrNrGrUrUrUrUrAr |
| | nucleotides | linkages (between n and n- | GrArGrCrUrArGrArArArUrArGrCrAr |
| | 2 phosphorodithioate | 1 and between n-1 and n-2) | ArGrUrUrArArArArUrArArGrGrCrUr |
| | linkages (between | | ArGrUrCrCrGrUrUrArUrCrArArCrUr |
| | first and second and | | UrGrArArArArArGrUrGrGrCrArCrCr |
| | between second and | | GrArGrUrCrGrGrUrGrCrUmU**mU** |
| | third) | | mU-3′ (SEQ ID NO: 23) |
| | 1 phosphorothioate | | |
| | linkage between third | | |
| | and fourth | | |
| | nucleotides | | |
| T | 2′-O-methyl | 2′-O-methyl modifications | 5′- |
| | modifications for first | for terminal 3 nucleotides | mN**mN**mN**rNrNrNrNrNrNrNrN |
| | 3 consecutive | 1 phosphorodithioate | rNrNrNrNrNrNrNrNrNrGrUrUrUrUrAr |
| | nucleotides | linkage (between n and n-1) | GrArGrCrUrArGrArArArUrArGrCrAr |
| | 3 phosphorodithioate | 1 phosphorothioate | ArGrUrUrArArArArUrArArGrGrCrUr |
| | linkages | linkage (between n-1 and | ArGrUrCrCrGrUrUrArUrCrArArCrUr |
| | | n-2) | UrGrArArArArArGrUrGrGrCrArCrCr |
| | | | GrArGrUrCrGrGrUrGrCrUmU*mU** |
| | | | mU-3′ (SEQ ID NO: 24) |
| U | 2′-O-methyl | 2′-O-methyl modifications | 5′- |
| | modifications for first | for terminal 5 nucleotides | mN**mN**mN**mN**rNrNrNrNrNr |
| | 4 consecutive | 4 phosphorodithioate | NrNrNrNrNrNrNrNrNrNrNrGrUrUrUr |
| | nucleotides | linkages (between n and n- | UrArGrArGrCrUrArGrArArArUrArGr |
| | 4 phosphorodithioate | 1, between n-1 and n-2, | CrArArGrUrUrArArArArUrArArGrGr |
| | linkages | between n-2 and n-3 and | CrUrArGrUrCrCrGrUrUrArUrCrArAr |
| | | between n-3 and n-4) | CrUrUrGrArArArArArGrUrGrGrCrAr |
| | | | CrCrGrArGrUrCrGrGrUrGmC**mU** |
| | | | mU**mU**mU-3′ (SEQ ID NO: 25) |
| V | 2′-O-methyl | 2′-O-methyl modifications | 5′- |
| | modifications for first | for n-1, n-2, n-3 and n-4. | mN**mN**mN**rNrNrNrNrNrNrNrN |
| | 3 consecutive | phosphorodithioate | rNrNrNrNrNrNrNrNrNrGrUrUrUrUrAr |
| | nucleotides | linkages (between n and n- | GrArGrCrUrArGrArArArUrArGrCrAr |
| | 3 phosphorodithioate | 1, between n-1 and n-2 | ArGrUrUrArArArArUrArArGrGrCrUr |
| | linkages | and between n-2 and n-3) | ArGrUrCrCrGrUrUrArUrCrArArCrUr |
| | | | UrGrArArArArArGrUrGrGrCrArCrCr |
| | | | GrArGrUrCrGrGrUrGrCmU**mU**m |
| | | | U**U-3′ (SEQ ID NO: 26) |

TABLE 11

| Name of | Modification Strategy at 5′ Terminus | Modification Strategy at 3′ Terminus |
|---|---|---|
| gRNA | Compared to Reference gRNA 3 | Compared to Reference gRNA 3 |
| B | Replace phosphorothioate linkages | Replace phosphorothioate linkages with |
| | with phosphorodithioate linkages | phosphorodithioate linkages |
| M | Replace phosphorothioate linkages | Replace phosphorothioate linkages with |
| | with phosphorodithioate linkages | phosphorodithioate linkages |
| | | Replace 2′-O-methyl modified nucleotides |
| | | with deoxyribose nucleotides |
| N | Replace phosphorothioate linkages | No changes |
| | with phosphorodithioate linkages | |
| | Replace 2′-O-methyl modified | |
| | nucleotides with deoxyribose | |
| | nucleotides | |
| O | No changes | Replace phosphorothioate linkages with |
| | | phosphorodithioate linkages |
| | | Replace 2′-O-methyl modified nucleotides |
| | | with deoxyribose nucleotides |
| P | Replace phosphorothioate linkages | No changes |
| | with phosphorodithioate linkages | |
| Q | No changes | Replace phosphorothioate linkages with |
| | | phosphorodithioate linkages |
| R | Replace phosphorothioate linkages | Replace phosphorothioate linkages with |
| | between second and third and third and | phosphorodithioate linkages |
| | fourth with phosphorodithioate | |
| | linkages | |
| S | Replace phosphorothioate linkages | Replace phosphorothioate linkages with |
| | between first and second and between | phosphorodithioate linkages |
| | second and third with | |
| | phosphorodithioate linkages | |
| T | Replace phosphorothioate linkages | Replace phosphorothioate linkage between |
| | with phosphorodithioate linkages | n and n−1 with phosphorodithioate linkage |
| U | Replace phosphorothioate linkages | Add two additional 2′-O-methyl modified |
| | with phosphorodithioate linkages and | nucleotides (at n−3 and n−4) |
| | add an additional phosphorodithioate | Replace phosphorothioate linkages with |
| | linkage (first four nucleotides have | phosphorodithioate linkages and add two |
| | phosphorodithioate linkages) | additional phosphorodithioate linkages (last |
| | | four nucleotides have phosphorodithioate |
| | | linkages) |
| V | Replace phosphorothioate linkages | Replace 2′-O-methyl modified nucleotides |
| | with phosphorodithioate linkages | by RNA at position n and Replace RNA at |
| | | position n−3 by 2′-O-methyl modified |
| | | nucleotides |
| | | Replace phosphorothioate linkages with |
| | | phosphorodithioate linkages and add an |
| | | additional phosphorodithioate linkages |

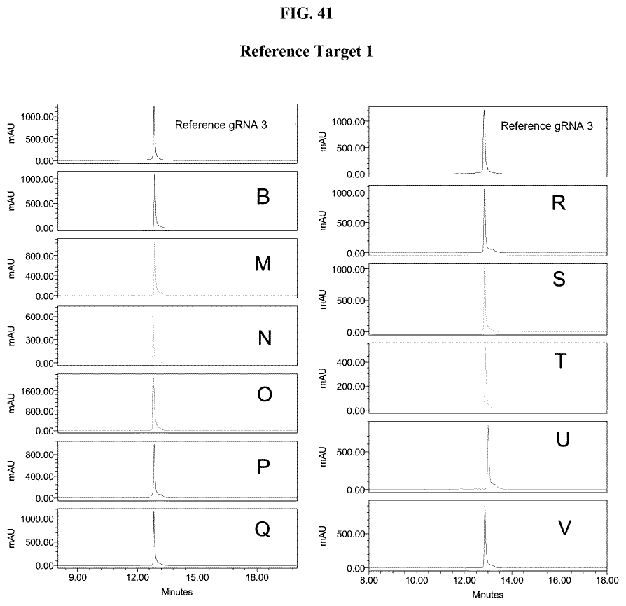

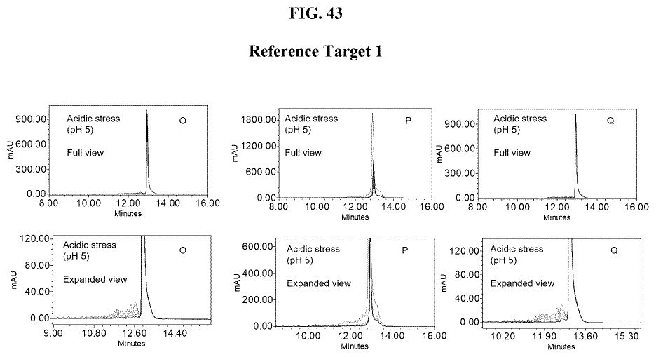

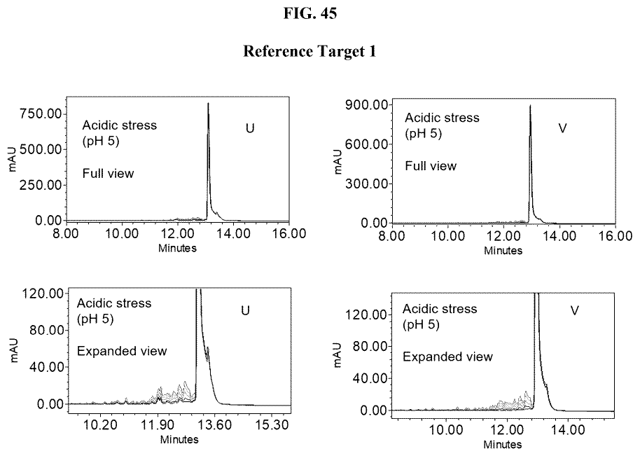

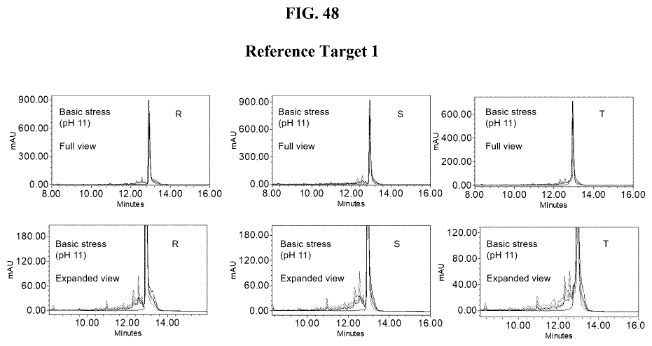

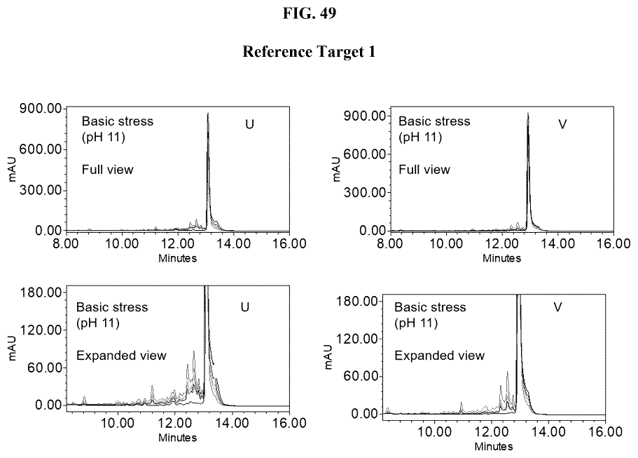

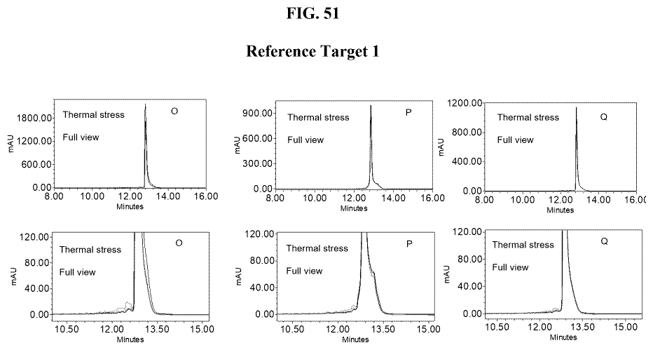

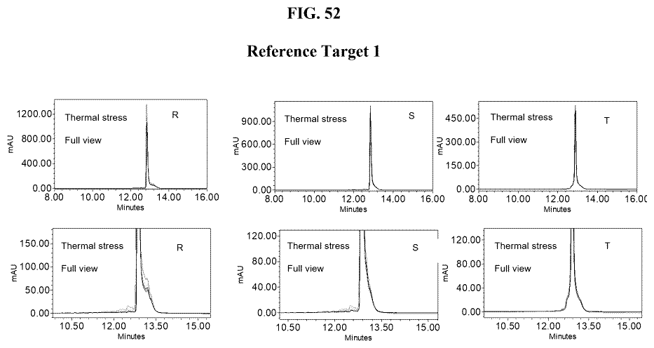

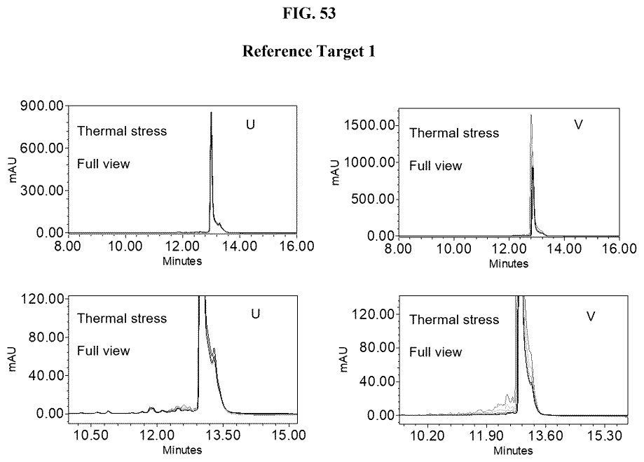

FIG. 41 and Table 12 show the initial purity profiles of Reference gRNA 3 and the gRNAs modified according to modification schemes B and M-V. Similar purity profiles were observed for gRNAs with modifications according to modification schemes B and M-V (Table 12 and FIG. 41). The results of the forced degradation studies of these modified gRNAs are shown in Table 12 and FIGS. 41-59. Experimental details of the conditions for the forced degradation studies are provided in Example 2.

TABLE 12
Purity Summary under Forced Degradation

| | Purity (area %) | | | | |
|---|---|---|---|---|---|
| Name of | | 0.3% H₂O₂, | pH 8, 40° | pH 5, 40° | pH 11, 40° |
| gRNA | T(0) | 40° C., T(5 d) | C., T(7 d) | C., T(7 d) | C., T(2 d) |
| Reference | 91.2 | 86.8 | 86.8 | 72.6 | 18.4 |
| gRNA 3 | | | | | |
| B | 92.5 | 83.8 | 89.7 | 78.8 | 40.6 |
| M | 88.1 | 76.0 | 85.1 | 73.5 | 49.2 |
| N | 82.7 | 79.1 | 79.1 | 69.5 | 44.6 |
| O | 93.4 | 78.4 | 89.8 | 75.7 | 59.5 |
| P | 88.9 | 77.1 | 86.0 | 74.3 | 73.1 |
| Q | 90.4 | 74.1 | 87.3 | 75.2 | 49.8 |
| R | 83.9 | 79.1 | 80.9 | 71.5 | 51.6 |
| S | 92.7 | 79.7 | 89.8 | 76.5 | 40.9 |
| T | 90.3 | 82.7 | 86.9 | 75.2 | 34.7 |
| U | 84.2 | 74.2 | 81.2 | 69.4 | 42.4 |
| V | 93.3 | 78.5 | 89.0 | 77.6 | 64.6 |

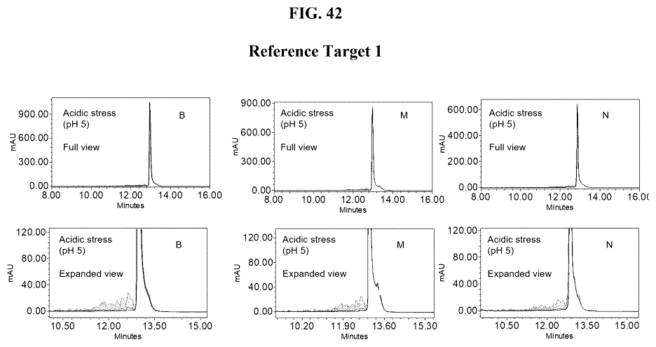

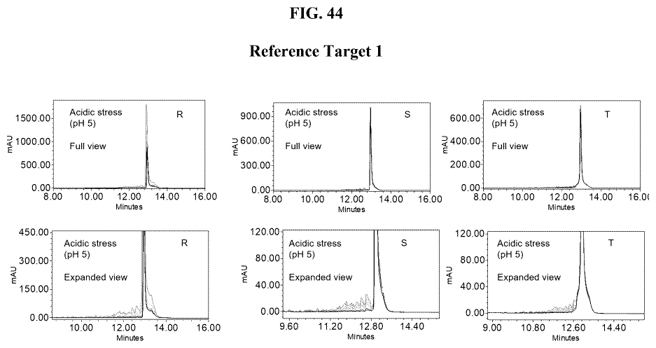

FIGS. 42-45 show the chromatograms of the gRNAs B and M-V under acidic stress (pH 5). As shown in FIG. 44, the gRNA modified according to scheme “T” appears to be the most resistant to acidic stress (pH 5) conditions. These data show that the inclusion of phosphorodithioate linkages at the 3′ termini of the gRNA results in increased stability under acidic stress conditions.

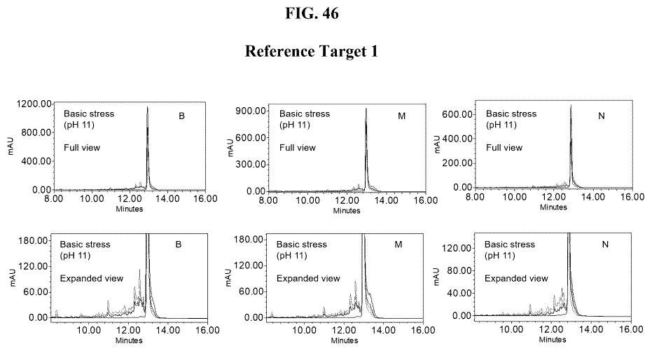

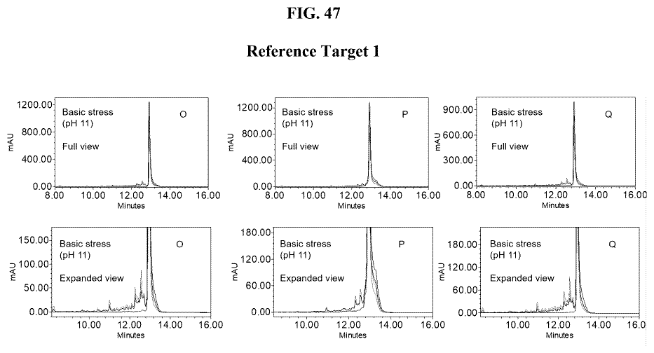

FIGS. 46-49 show the chromatograms of the gRNAs B and M-V under basic stress (pH 11). As shown in FIG. 47, the gRNA modified according to scheme “P” appears to be the most resistant to basic stress (pH 11) conditions. These data show that the inclusion of phosphorodithioate linkages at the 5′ termini of the gRNA results in increased stability under basic stress conditions.

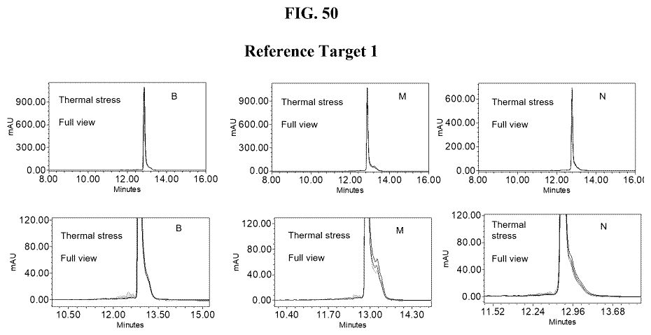

FIGS. 50-53 show the chromatograms of the gRNAs B and M-V under thermal stress. Most gRNAs were resistant to thermal degradation.

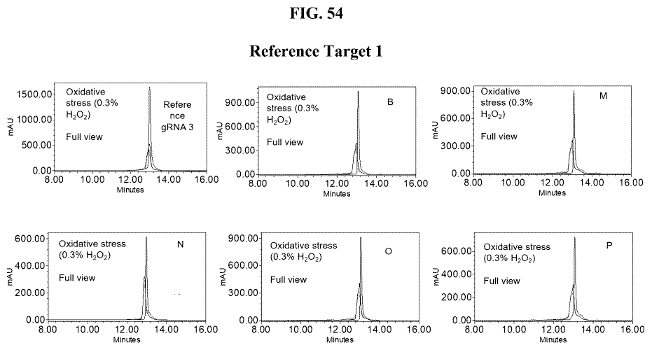

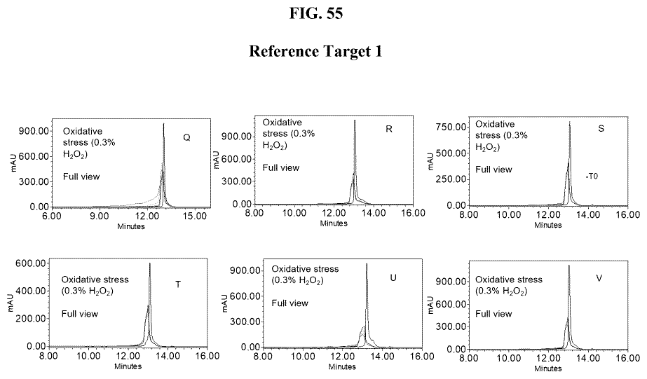

FIGS. 54-55 shows the chromatograms of the gRNAs B and M-V under oxidative stress (0.3% H₂O₂). Most gRNAs were susceptible to oxidative degradation.

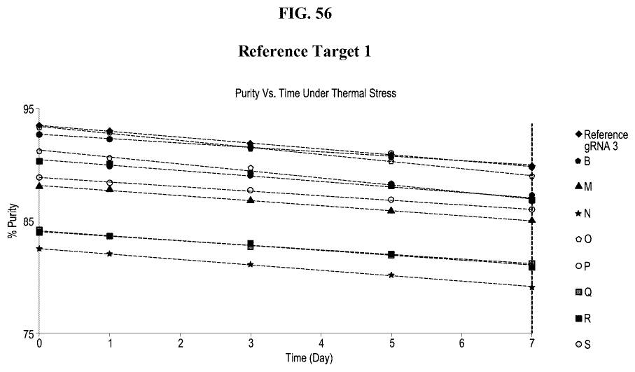

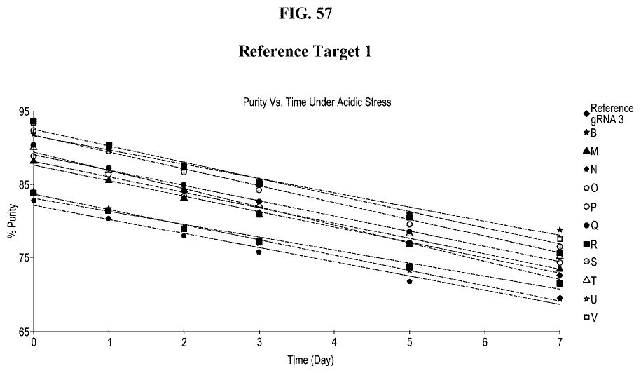

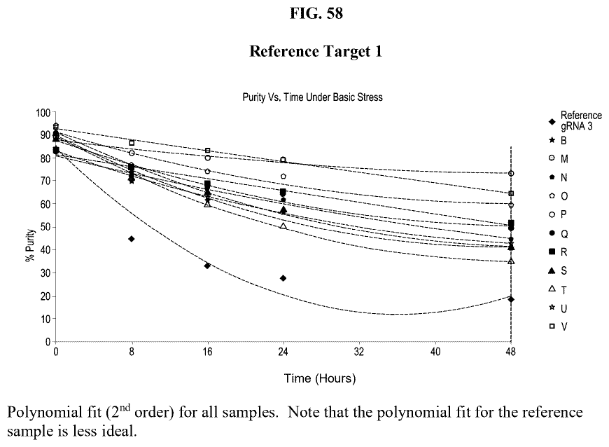

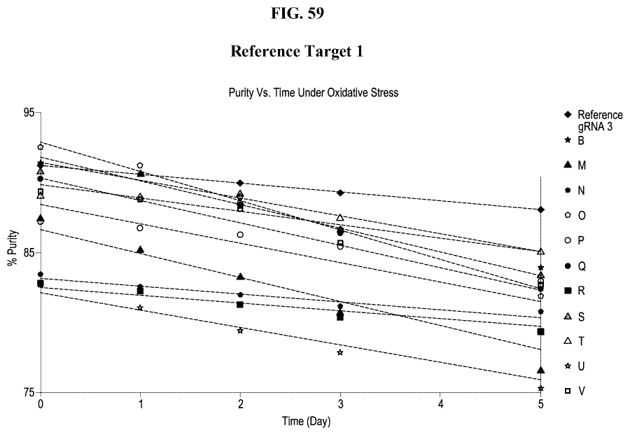

FIG. 56 shows the changes in purity over a 7-day time period during thermal stress. FIG. 57 shows the changes in purity over a 7-day time period during acidic stress (pH 5). FIG. 58 shows the changes in purity over a 48-hour period during basic stress (pH 11). FIG. 59 shows the changes in purity over a 5-day time period under oxidative stress (0.3% H₂O₂).

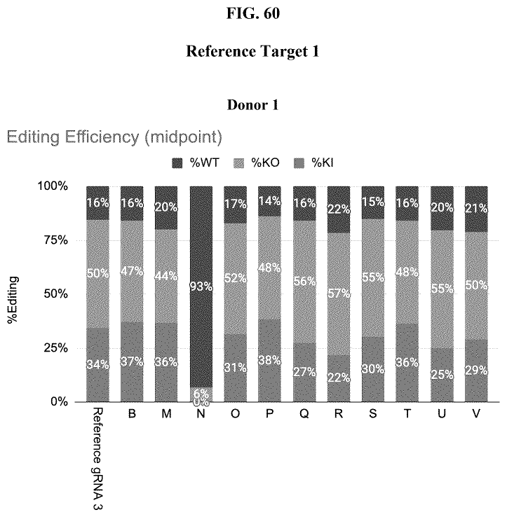

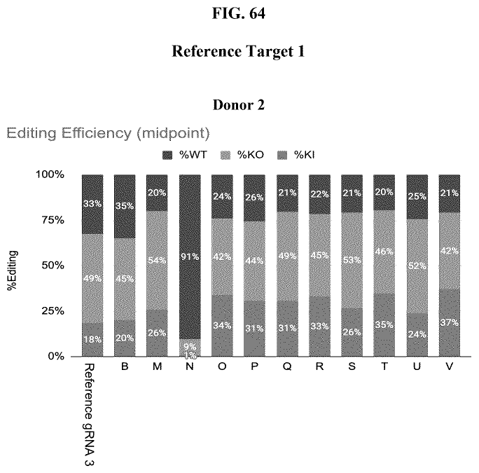

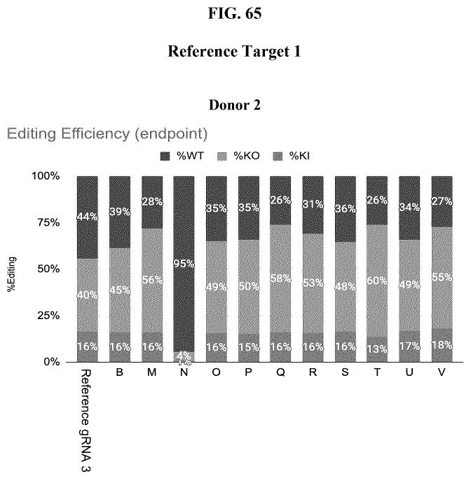

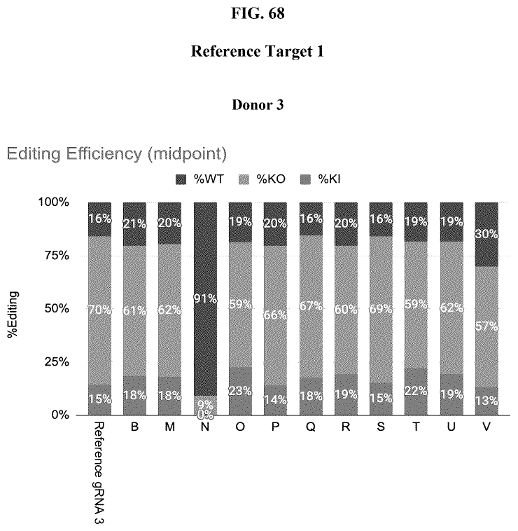

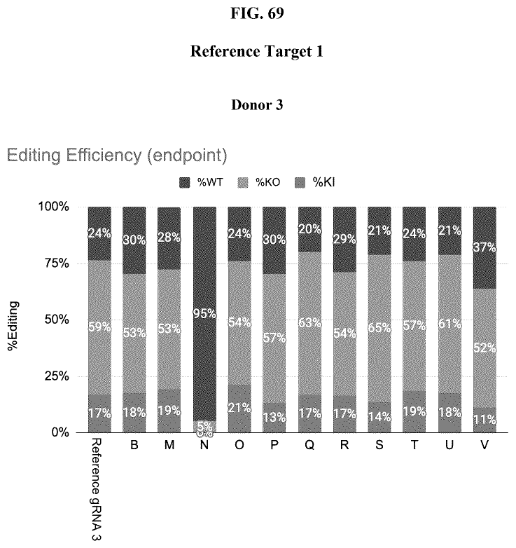

Further analysis of the characteristics of gRNAs targeting Reference Target 1 and modified according to modification schemes B and M-V was performed in three donor cell lines, donor 1, donor 2 and donor 3. Using the Lonza 4D system, the editing efficiency, cell expansion and cell phenotype of these gRNAs were analyzed using about 3.0×10⁷ cells/tfx at a 100 μL scale. In donor 1 (FIGS. 60 and 61), donor 2 (FIGS. 64 and 65) and donor 3 (FIGS. 68 and 69), low editing was observed with the gRNA modified according to scheme “N” at the midpoint and endpoint. In addition, similar percentages were observed for knock-in (KI) and knock-out (KO) editing for each modified gRNA in all 3 donors except for the gRNA modified using scheme “N.” As shown in FIGS. 61, 65 and 69, improved editing efficiency was observed with the gRNAs modified according to modification scheme “S” and “T” compared to the reference gRNA. In particular, the number of wild-type cells for reference gRNA (i.e., cells that were not edited by the gRNA) was observed to be 22%-39%, whereas the number of wild-type cells for S” and “T” modified gRNAs was observed to be between 18%-36% at the end timepoint of the study. In addition, the percentages for KO editing were between 51%-78% for reference gRNA 3 compared to 64%-82% for the “S” and “T” modified gRNAs and the percentages for KI editing were between 16%-23% for the reference gRNA 3 compared to 13%-28% for the “S” and “T” modified gRNAs at the end timepoint of the study. These results show that “S” and “T” modified gRNAs exhibited comparable or improved editing efficiency compared to reference gRNA 3.

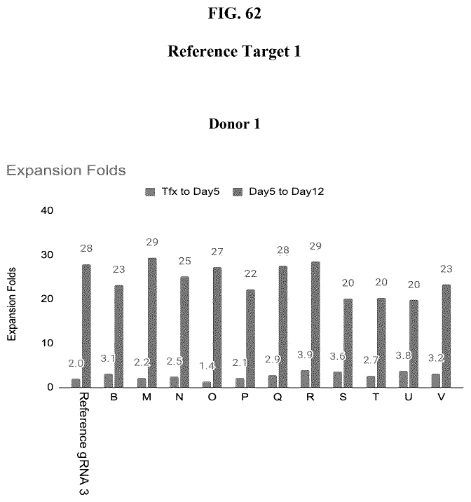

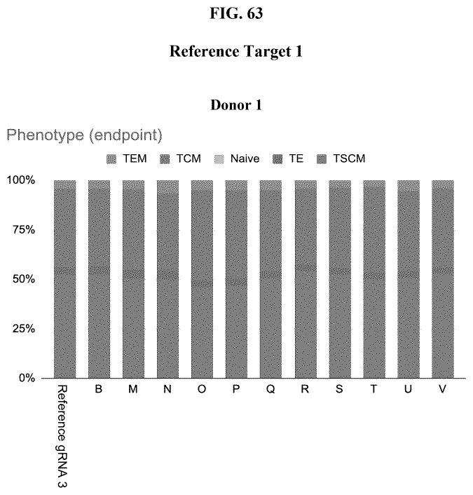

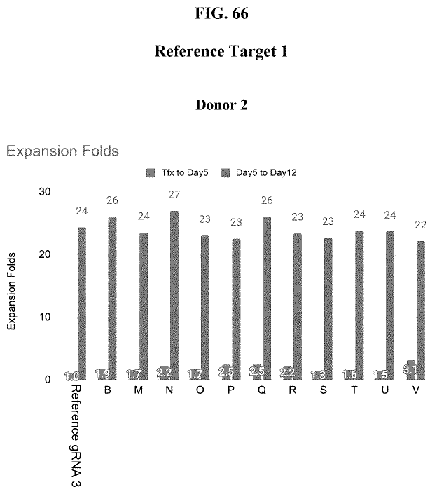

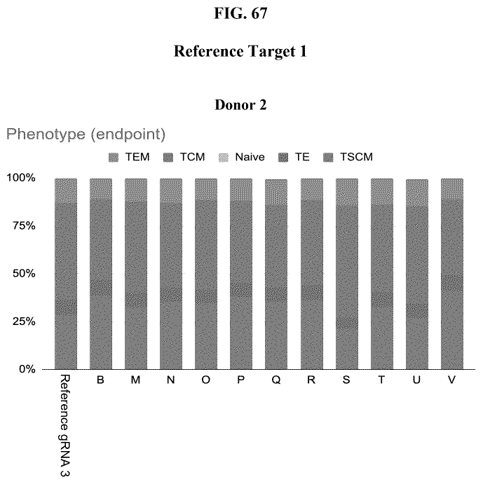

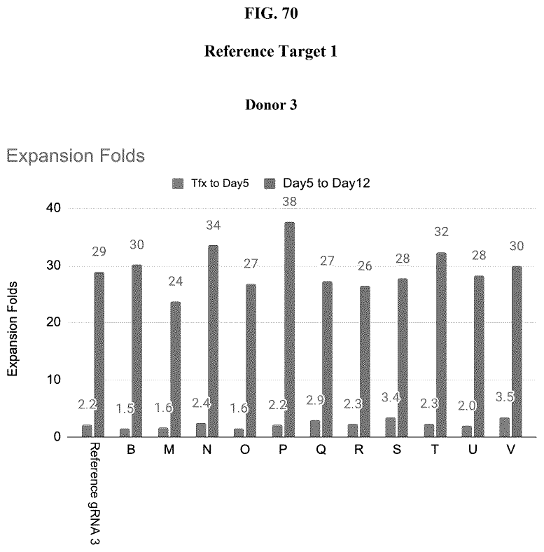

Similar cell expansion rates were observed for all modified gRNAs in donor 1 (FIG. 62), donor 2 (FIG. 66) and donor 3 (FIG. 70).

All publications, patents and patent applications mentioned herein are hereby incorporated by reference in their entirety as if each individual publication, patent or patent application was specifically and individually indicated to be incorporated by reference. In case of conflict, the present application, including any definitions herein, will control.

1. A guide RNA molecule comprising:
a phosphorodithioate linkage between the first and second nucleotides, the second and third nucleotides, and the third and fourth nucleotides at the 5′ terminus of the guide RNA molecule and a phosphorodithioate linkage between the 3′ terminal (“n”) nucleotide and the n−1 nucleotide and the n−1 and n−2 nucleotides of the guide RNA molecule,
wherein the first nucleotide, the second nucleotide, and the third nucleotide at the 5′ terminus of the guide RNA molecule comprise a 2′-O-methyl modified nucleotide or comprise a deoxyribose nucleotide, and
wherein the n, n−1, and n−2 nucleotides of the guide RNA molecule comprise a 2′-O-methyl modified nucleotide or comprise a deoxyribose nucleotide.

2-13. (canceled)

14. The guide RNA molecule of claim 1, wherein the fourth nucleotide at the 5′ terminus of the guide RNA molecule comprises a modification selected from the group consisting of:
(a) a 2′-fluoro modified nucleotide;
(b) a 2′-O-methyl modified nucleotide;
(c) a 2′-O-(2-methoxyethyl) modified nucleotide;
(d) a locked nucleic acid (LNA); and
(e) a deoxyribose nucleotide.

15. (canceled)

16. (canceled)

17. The guide RNA molecule of claim 1, wherein the guide RNA molecule comprises four consecutive 2′-O-methyl modified nucleotides at the first four nucleotides at the 5′ terminus of the guide RNA molecule.

18-26. (canceled)

27. The guide RNA molecule of claim 1, wherein (i) the n−3 nucleotide or (ii) the n−4 nucleotide, of the guide RNA molecule each comprises a modification selected from the group consisting of:
(a) a 2′-fluoro modified nucleotide;
(b) a 2′-O-methyl modified nucleotide;
(c) a 2′-O-(2-methoxyethyl) modified nucleotide;
(d) a locked nucleic acid (LNA); and
(e) a deoxyribose nucleotide.

28. (canceled)

29. (canceled)

30. The guide RNA molecule of claim 1, wherein the n, n−1, n−2, n−3 and n−4 nucleotides of the guide RNA molecule each are 2′-O-methyl modified nucleotides.

31-38. (canceled)

39. A composition comprising a guide RNA molecule of claim 1.

40. The composition of claim 39 further comprising an RNA-guided nuclease.

41. The composition of claim 39 further comprising a nucleic acid encoding an RNA-guided nuclease.

42. The composition of claim 40, wherein the RNA-guided nuclease is a Cas protein.

43. (canceled)

44. (canceled)

45. A ribonucleoprotein (RNP) complex comprising a guide RNA molecule of claim 1 and an RNA-guided nuclease.

46. The RNP complex of claim 45, wherein the RNA-guided nuclease is a Cas protein.

47. The RNP complex of claim 46, wherein the Cas protein is selected from the group consisting of a Cas9, a Cas12, a Cas13 and a combination thereof.

48. A cell comprising a composition of claim 39.

49. A cell comprising an RNP complex of claim 45.

50. A method of modifying a cell comprising contacting the cell with the composition of claim 39.

51. The method of claim 50, wherein said contacting comprises introducing the composition into the cell by electroporation.

52. A method of treating a subject in need thereof, comprising:
(a) modifying a cell of the subject ex vivo by contacting the cell with the composition of claim 39; and
(b) returning the modified cell to the subject.

53. A gene editing system comprising:
(a) one or more guide RNA molecules of claim 1; and
(b) an RNA-guided nuclease.

54. A method of modifying a cell comprising contacting the cell with the RNP complex of claim 45.

55. A method of treating a subject in need thereof, comprising:
(a) modifying a cell of the subject ex vivo by contacting the cell with the RNP complex of claim 45; and
(b) returning the modified cell to the subject.


SEQUENCE LISTING (26 sequences)

SEQ ID NO: 1 - RNA, 100 nt, synthetic construct
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide
nnnnnnnnnnnnnnnnnnnngttttagagctagaaatagcaagttaaaataaggctagtccgttatcaacttgaaaaagtggcaccgagtcggtgctttt

SEQ ID NO: 2 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 97: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage

SEQ ID NO: 3 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 4 - RNA, 100 nt, synthetic construct; identical to SEQ ID NO: 1
  source 1..100: mol_type = other RNA
  misc_difference 1: note = Locked adenosine, cytidine, uridine, guanosine, unknown or other nucleotide with phosphorothioate linkage
  misc_difference 2: note = Locked adenosine, cytidine, uridine, guanosine, unknown or other nucleotide with phosphorothioate linkage
  misc_difference 3: note = Locked adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = Locked nucleic acid with phosphorothioate linkage
  modified_base 99: mod_base = OTHER; note = Locked nucleic acid with phosphorothioate linkage
  modified_base 100: mod_base = OTHER; note = Locked nucleic acid

SEQ ID NO: 5 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorodithioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorodithioate linkage
  modified_base 99: mod_base = OTHER; note = 2'-O-methyl modified nucleotide with phosphorodithioate linkage
  modified_base 100: mod_base = OTHER; note = 2'-O-methyl modified nucleotide

SEQ ID NO: 6 - RNA, 100 nt, synthetic construct; identical to SEQ ID NO: 1
  source 1..100: mol_type = other RNA
  misc_difference 1: note = 2'-O-(2-methoxyethyl) modified adenosine, cytidine, uridine, guanosine, unknown or other nucleotide with phosphorothioate linkage
  misc_difference 2: note = 2'-O-(2-methoxyethyl) modified adenosine, cytidine, uridine, guanosine, unknown or other nucleotide with phosphorothioate linkage
  misc_difference 3: note = 2'-O-(2-methoxyethyl) modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-(2-methoxyethyl) modified nucleotide with phosphorothioate linkage
  modified_base 99: mod_base = OTHER; note = 2'-O-(2-methoxyethyl) modified nucleotide with phosphorothioate linkage
  modified_base 100: mod_base = OTHER; note = 2'-O-(2-methoxyethyl) modified nucleotide

SEQ ID NO: 7 - RNA, 100 nt, synthetic construct; identical to SEQ ID NO: 1
  source 1..100: mol_type = other RNA
  misc_difference 1: note = 2'-fluoro modified adenosine, cytidine, uridine, guanosine, unknown or other nucleotide with phosphorothioate linkage
  misc_difference 2: note = 2'-fluoro modified adenosine, cytidine, uridine, guanosine, unknown or other nucleotide with phosphorothioate linkage
  misc_difference 3: note = 2'-fluoro modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-fluoro modified nucleotide with phosphorothioate linkage
  modified_base 99: mod_base = OTHER; note = 2'-fluoro modified nucleotide with phosphorothioate linkage
  modified_base 100: mod_base = OTHER; note = 2'-fluoro modified nucleotide

SEQ ID NO: 8 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = 2'-fluoro modified adenosine, cytidine, uridine, guanosine, unknown or other nucleotide
  misc_difference 5: note = 2'-fluoro modified adenosine, cytidine, uridine, guanosine, unknown or other nucleotide
  misc_difference 6: note = 2'-fluoro modified adenosine, cytidine, uridine, guanosine, unknown or other nucleotide
  misc_difference 7: note = 2'-fluoro modified adenosine, cytidine, uridine, guanosine, unknown or other nucleotide
  misc_difference 8: note = 2'-fluoro modified adenosine, cytidine, uridine, guanosine, unknown or other nucleotide
  misc_difference 9: note = 2'-fluoro modified adenosine, cytidine, uridine, guanosine, unknown or other nucleotide
  misc_difference 10: note = 2'-fluoro modified adenosine, cytidine, uridine, guanosine, unknown or other nucleotide
  misc_difference 11: note = 2'-fluoro modified adenosine, cytidine, uridine, guanosine, unknown or other nucleotide
  misc_difference 12: note = 2'-fluoro modified adenosine, cytidine, uridine, guanosine, unknown or other nucleotide
  misc_difference 13: note = 2'-fluoro modified adenosine, cytidine, uridine, guanosine, unknown or other nucleotide
  misc_difference 14: note = 2'-fluoro modified adenosine, cytidine, uridine, guanosine, unknown or other nucleotide
  misc_difference 15: note = 2'-fluoro modified adenosine, cytidine, uridine, guanosine, unknown or other nucleotide
  misc_difference 16: note = 2'-fluoro modified adenosine, cytidine, uridine, guanosine, unknown or other nucleotide
  misc_difference 17: note = 2'-fluoro modified adenosine, cytidine, uridine, guanosine, unknown or other nucleotide
  misc_difference 18: note = 2'-fluoro modified adenosine, cytidine, uridine, guanosine, unknown or other nucleotide
  misc_difference 19: note = 2'-fluoro modified adenosine, cytidine, uridine, guanosine, unknown or other nucleotide
  misc_difference 20: note = 2'-fluoro modified adenosine, cytidine, uridine, guanosine, unknown or other nucleotide
  modified_base 21: mod_base = OTHER; note = 2'-fluoro modified nucleotide
  modified_base 22: mod_base = OTHER; note = 2'-fluoro modified nucleotide
  modified_base 23: mod_base = OTHER; note = 2'-fluoro modified nucleotide
  modified_base 24: mod_base = OTHER; note = 2'-fluoro modified nucleotide
  modified_base 25: mod_base = OTHER; note = 2'-fluoro modified nucleotide
  modified_base 26: mod_base = OTHER; note = 2'-fluoro modified nucleotide
  modified_base 27: mod_base = OTHER; note = 2'-fluoro modified nucleotide
  modified_base 28: mod_base = OTHER; note = 2'-fluoro modified nucleotide
  modified_base 29: mod_base = OTHER; note = 2'-fluoro modified nucleotide
  modified_base 30: mod_base = OTHER; note = 2'-fluoro modified nucleotide
  modified_base 31: mod_base = OTHER; note = 2'-fluoro modified nucleotide
  modified_base 32: mod_base = OTHER; note = 2'-fluoro modified nucleotide
  modified_base 33: mod_base = OTHER; note = 2'-fluoro modified nucleotide
  modified_base 34: mod_base = OTHER; note = 2'-fluoro modified nucleotide
  modified_base 35: mod_base = OTHER; note = 2'-fluoro modified nucleotide
  modified_base 36: mod_base = OTHER; note = 2'-fluoro modified nucleotide
  modified_base 37: mod_base = OTHER; note = 2'-fluoro modified nucleotide
  modified_base 38: mod_base = OTHER; note = 2'-fluoro modified nucleotide
  modified_base 39: mod_base = OTHER; note = 2'-fluoro modified nucleotide
  modified_base 40: mod_base = OTHER; note = 2'-fluoro modified nucleotide
  modified_base 41: mod_base = OTHER; note = 2'-fluoro modified nucleotide
  modified_base 42: mod_base = OTHER; note = 2'-fluoro modified nucleotide
  modified_base 43: mod_base = OTHER; note = 2'-fluoro modified nucleotide
  modified_base 44: mod_base = OTHER; note = 2'-fluoro modified nucleotide
  modified_base 45: mod_base = OTHER; note = 2'-fluoro modified nucleotide
  modified_base 46: mod_base = OTHER; note = 2'-fluoro modified nucleotide
  modified_base 47: mod_base = OTHER; note = 2'-fluoro modified nucleotide
  modified_base 48: mod_base = OTHER; note = 2'-fluoro modified nucleotide
  modified_base 49: mod_base = OTHER; note = 2'-fluoro modified nucleotide
  modified_base 50: mod_base = OTHER; note = 2'-fluoro modified nucleotide
  modified_base 51: mod_base = OTHER; note = 2'-fluoro modified nucleotide
  modified_base 52: mod_base = OTHER; note = 2'-fluoro modified nucleotide
  modified_base 53: mod_base = OTHER; note = 2'-fluoro modified nucleotide
  modified_base 54: mod_base = OTHER; note = 2'-fluoro modified nucleotide
  modified_base 55: mod_base = OTHER; note = 2'-fluoro modified nucleotide
  modified_base 56: mod_base = OTHER; note = 2'-fluoro modified nucleotide
  modified_base 57: mod_base = OTHER; note = 2'-fluoro modified nucleotide
  modified_base 58: mod_base = OTHER; note = 2'-fluoro modified nucleotide
  modified_base 59: mod_base = OTHER; note = 2'-fluoro modified nucleotide
  modified_base 60: mod_base = OTHER; note = 2'-fluoro modified nucleotide
  modified_base 61: mod_base = OTHER; note = 2'-fluoro modified nucleotide
  modified_base 62: mod_base = OTHER; note = 2'-fluoro modified nucleotide
  modified_base 63: mod_base = OTHER; note = 2'-fluoro modified nucleotide
  modified_base 64: mod_base = OTHER; note = 2'-fluoro modified nucleotide
  modified_base 65: mod_base = OTHER; note = 2'-fluoro modified nucleotide
  modified_base 66: mod_base = OTHER; note = 2'-fluoro modified nucleotide
  modified_base 67: mod_base = OTHER; note = 2'-fluoro modified nucleotide
  modified_base 68: mod_base = OTHER; note = 2'-fluoro modified nucleotide
  modified_base 69: mod_base = OTHER; note = 2'-fluoro modified nucleotide
  modified_base 70: mod_base = OTHER; note = 2'-fluoro modified nucleotide
  modified_base 71: mod_base = OTHER; note = 2'-fluoro modified nucleotide
  modified_base 72: mod_base = OTHER; note = 2'-fluoro modified nucleotide
  modified_base 73: mod_base = OTHER; note = 2'-fluoro modified nucleotide
  modified_base 74: mod_base = OTHER; note = 2'-fluoro modified nucleotide
  modified_base 75: mod_base = OTHER; note = 2'-fluoro modified nucleotide
  modified_base 76: mod_base = OTHER; note = 2'-fluoro modified nucleotide
  modified_base 77: mod_base = OTHER; note = 2'-fluoro modified nucleotide
  modified_base 78: mod_base = OTHER; note = 2'-fluoro modified nucleotide
  modified_base 79: mod_base = OTHER; note = 2'-fluoro modified nucleotide
  modified_base 80: mod_base = OTHER; note = 2'-fluoro modified nucleotide
  modified_base 81: mod_base = OTHER; note = 2'-fluoro modified nucleotide
  modified_base 82: mod_base = OTHER; note = 2'-fluoro modified nucleotide
  modified_base 83: mod_base = OTHER; note = 2'-fluoro modified nucleotide
  modified_base 84: mod_base = OTHER; note = 2'-fluoro modified nucleotide
  modified_base 85: mod_base = OTHER; note = 2'-fluoro modified nucleotide
  modified_base 86: mod_base = OTHER; note = 2'-fluoro modified nucleotide
  modified_base 87: mod_base = OTHER; note = 2'-fluoro modified nucleotide
  modified_base 88: mod_base = OTHER; note = 2'-fluoro modified nucleotide
  modified_base 89: mod_base = OTHER; note = 2'-fluoro modified nucleotide
  modified_base 90: mod_base = OTHER; note = 2'-fluoro modified nucleotide
  modified_base 91: mod_base = OTHER; note = 2'-fluoro modified nucleotide
  modified_base 92: mod_base = OTHER; note = 2'-fluoro modified nucleotide
  modified_base 93: mod_base = OTHER; note = 2'-fluoro modified nucleotide
  modified_base 94: mod_base = OTHER; note = 2'-fluoro modified nucleotide
  modified_base 95: mod_base = OTHER; note = 2'-fluoro modified nucleotide
  modified_base 96: mod_base = OTHER; note = 2'-fluoro modified nucleotide
  modified_base 97: mod_base = OTHER; note = 2'-fluoro modified nucleotide
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 9 - DNA, 100 bp, synthetic construct
  source 1..100: mol_type = other DNA
  misc_difference 1: note = Adenosine, cytidine, thymidine, guanosine, unknown or other nucleotide with phosphorodithioate linkage
  misc_difference 2: note = Adenosine, cytidine, thymidine, guanosine, unknown or other nucleotide with phosphorodithioate linkage
  misc_difference 3: note = Adenosine, cytidine, thymidine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = Nucleotide with phosphorodithioate linkage
  modified_base 99: mod_base = OTHER; note = Nucleotide with phosphorodithioate linkage
  misc_feature 1..3: note = DNA
  misc_feature 4..97: note = RNA
  misc_feature 98..100: note = DNA
nnnnnnnnnnnnnnnnnnnngttttagagctagaaatagcaagttaaaataaggctagtccgttatcaacttgaaaaagtggcaccgagtcggtgctttt

SEQ ID NO: 10 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide
  misc_difference 2: note = 2'-O-methyl modified adenosine, cytidine, uridine, guanosine, unknown or other nucleotide
  misc_difference 3: note = 2'-O-methyl modified adenosine, cytidine, uridine, guanosine, unknown or other nucleotide
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide
  modified_base 99: mod_base = OTHER; note = 2'-O-methyl modified nucleotide
  modified_base 100: mod_base = OTHER; note = 2'-O-methyl modified nucleotide

SEQ ID NO: 11 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 22: mod_base = OTHER; note = Nucleotide with phosphorothioate linkage
  modified_base 23: mod_base = OTHER; note = Nucleotide with phosphorothioate linkage
  modified_base 24: mod_base = OTHER; note = Nucleotide with phosphorothioate linkage
  modified_base 25: mod_base = OTHER; note = Nucleotide with phosphorothioate linkage
  modified_base 26: mod_base = OTHER; note = Nucleotide with phosphorothioate linkage
  modified_base 27: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
  modified_base 47: mod_base = OTHER; note = Nucleotide with phosphorothioate linkage
  modified_base 49: mod_base = OTHER; note = Nucleotide with phosphorothioate linkage
  modified_base 51: mod_base = OTHER; note = Nucleotide with phosphorothioate linkage
  modified_base 58: mod_base = OTHER; note = Nucleotide with phosphorothioate linkage
  modified_base 59: mod_base = OTHER; note = Nucleotide with phosphorothioate linkage
  modified_base 62: mod_base = OTHER; note = Nucleotide with phosphorothioate linkage
  modified_base 63: mod_base = OTHER; note = Nucleotide with phosphorothioate linkage
  modified_base 64: mod_base = OTHER; note = Nucleotide with phosphorothioate linkage
  modified_base 65: mod_base = OTHER; note = Nucleotide with phosphorothioate linkage
  modified_base 68: mod_base = OTHER; note = Nucleotide with phosphorothioate linkage
  modified_base 69: mod_base = OTHER; note = Nucleotide with phosphorothioate linkage
  modified_base 82: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 12 - DNA, 100 bp, synthetic construct; identical to SEQ ID NO: 9
  source 1..100: mol_type = other DNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = N can be a,g,c,t
  misc_difference 5: note = N can be a,g,c,t
  misc_difference 6: note = N can be a,g,c,t
  misc_difference 7: note = N can be a,g,c,t
  misc_difference 8: note = N can be a,g,c,t
  misc_difference 9: note = N can be a,g,c,t
  misc_difference 10: note = N can be a,g,c,t
  misc_difference 11: note = N can be a,g,c,t
  misc_difference 12: note = N can be a,g,c,t
  misc_difference 13: note = N can be a,g,c,t
  misc_difference 14: note = N can be a,g,c,t
  misc_difference 15: note = N can be a,g,c,t
  misc_difference 16: note = N can be a,g,c,t
  misc_difference 17: note = N can be a,g,c,t
  misc_difference 18: note = N can be a,g,c,t
  misc_difference 19: note = N can be a,g,c,t
  misc_difference 20: note = N can be a,g,c,t
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide
  misc_feature 1..3: note = RNA
  misc_feature 4..20: note = DNA
  misc_feature 21..100: note = RNA

SEQ ID NO: 13 - RNA, 100 nt, synthetic construct; identical to SEQ ID NO: 1
  source 1..100: mol_type = other RNA
  misc_difference 1: note = Adenosine, cytidine, uridine, guanosine, unknown or other nucleotide with phosphorothioate linkage
  misc_difference 2: note = Adenosine, cytidine, uridine, guanosine, unknown or other nucleotide with phosphorothioate linkage
  misc_difference 3: note = Adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = Nucleotide with phosphorothioate linkage
  modified_base 99: mod_base = OTHER; note = Nucleotide with phosphorothioate linkage

SEQ ID NO: 14 - DNA, 100 bp, synthetic construct; identical to SEQ ID NO: 9
  source 1..100: mol_type = other DNA
  misc_difference 1: note = Adenosine, cytidine, thymidine, guanosine, unknown or other nucleotide with phosphorodithioate linkage
  misc_difference 2: note = Adenosine, cytidine, thymidine, guanosine, unknown or other nucleotide with phosphorodithioate linkage
  misc_difference 3: note = Adenosine, cytidine, thymidine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide
  misc_feature 1..3: note = DNA
  misc_feature 4..100: note = RNA

SEQ ID NO: 15 - RNA, 100 nt, synthetic construct; identical to SEQ ID NO: 1
  source 1..100: mol_type = other RNA
  misc_difference 1: note = N can be a,g,c,u
  misc_difference 2: note = N can be a,g,c,u
  misc_difference 3: note = N can be a,g,c,u
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 16 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorodithioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorodithioate linkage
  modified_base 99: mod_base = OTHER; note = 2'-O-methyl modified nucleotide with phosphorodithioate linkage
  modified_base 100: mod_base = OTHER; note = 2'-O-methyl modified nucleotide

SEQ ID NO: 17 - DNA, 100 bp, synthetic construct; identical to SEQ ID NO: 9
  source 1..100: mol_type = other DNA
  misc_difference 1: note = 2'-O-methyl modified adenosine, cytidine, uridine, guanosine, unknown or other nucleotide with phosphorodithioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = Nucleotide with phosphorodithioate linkage
  modified_base 99: mod_base = OTHER; note = Nucleotide with phosphorodithioate linkage
  misc_feature 1..97: note = RNA
  misc_feature 98..100: note = DNA

SEQ ID NO: 18 - DNA, 100 bp, synthetic construct; identical to SEQ ID NO: 9
  source 1..100: mol_type = other DNA
  misc_difference 1: note = Adenosine, cytidine, thymidine, guanosine, unknown or other nucleotide with phosphorodithioate linkage
  misc_difference 2: note = Adenosine, cytidine, thymidine, guanosine, unknown or other nucleotide with phosphorodithioate linkage
  misc_difference 3: note = Adenosine, cytidine, thymidine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide
  misc_feature 1..3: note = DNA
  misc_feature 4..100: note = RNA

SEQ ID NO: 19 - DNA, 100 bp, synthetic construct; identical to SEQ ID NO: 9
  source 1..100: mol_type = other DNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = Nucleotide with phosphorodithioate linkage
  modified_base 99: mod_base = OTHER; note = Nucleotide with phosphorodithioate linkage
  misc_feature 1..97: note = RNA
  misc_feature 98..100: note = DNA

SEQ ID NO: 20 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorodithioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 21 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorodithioate linkage
  modified_base 99: mod_base = OTHER; note = 2'-O-methyl modified nucleotide with phosphorodithioate linkage
  modified_base 100: mod_base = OTHER; note = 2'-O-methyl modified nucleotide

SEQ ID NO: 22 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorodithioate linkage
  modified_base 99: mod_base = OTHER; note = 2'-O-methyl modified nucleotide with phosphorodithioate linkage
  modified_base 100: mod_base = OTHER; note = 2'-O-methyl modified nucleotide

SEQ ID NO: 23 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorodithioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorothioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorodithioate linkage
  modified_base 99: mod_base = OTHER; note = 2'-O-methyl modified nucleotide with phosphorodithioate linkage
  modified_base 100: mod_base = OTHER; note = 2'-O-methyl modified nucleotide

SEQ ID NO: 24 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorodithioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorodithioate linkage
  modified_base 100: mod_base = OTHER; note = 2'-O-methyl modified nucleotide

SEQ ID NO: 25 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorodithioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorodithioate linkage
  misc_difference 4: note = 2'-O-methyl modified adenosine, cytidine, uridine, guanosine, unknown or other nucleotide with phosphorodithioate linkage
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 96: mod_base = OTHER; note = 2'-O-methyl modified nucleotide with phosphorodithioate linkage
  modified_base 97: mod_base = OTHER; note = 2'-O-methyl modified nucleotide with phosphorodithioate linkage
  modified_base 98: mod_base = OTHER; note = 2'-O-methyl modified nucleotide with phosphorodithioate linkage
  modified_base 99: mod_base = OTHER; note = 2'-O-methyl modified nucleotide with phosphorodithioate linkage
  modified_base 100: mod_base = OTHER; note = 2'-O-methyl modified nucleotide

SEQ ID NO: 26 - RNA, 100 nt, synthetic construct; identical to SEQ ID NO: 1
  source 1..100: mol_type = other RNA
  misc_difference 1: note = 2'-O-methyl modified adenosine, cytidine, uridine, guanosine, unknown or other nucleotide with phosphorodithioate linkage
  misc_difference 2: note = 2'-O-methyl modified adenosine, cytidine, uridine, guanosine, unknown or other nucleotide with phosphorodithioate linkage
  misc_difference 3: note = 2'-O-methyl modified adenosine, cytidine, uridine, guanosine, unknown or other nucleotide with phosphorodithioate linkage
  misc_difference 4: note = N can be a,g,c,u
  misc_difference 5: note = N can be a,g,c,u
  misc_difference 6: note = N can be a,g,c,u
  misc_difference 7: note = N can be a,g,c,u
  misc_difference 8: note = N can be a,g,c,u
  misc_difference 9: note = N can be a,g,c,u
  misc_difference 10: note = N can be a,g,c,u
  misc_difference 11: note = N can be a,g,c,u
  misc_difference 12: note = N can be a,g,c,u
  misc_difference 13: note = N can be a,g,c,u
  misc_difference 14: note = N can be a,g,c,u
  misc_difference 15: note = N can be a,g,c,u
  misc_difference 16: note = N can be a,g,c,u
  misc_difference 17: note = N can be a,g,c,u
  misc_difference 18: note = N can be a,g,c,u
  misc_difference 19: note = N can be a,g,c,u
  misc_difference 20: note = N can be a,g,c,u
  modified_base 97: mod_base = OTHER; note = 2'-O-methyl modified nucleotide with phosphorodithioate linkage
  modified_base 98: mod_base = OTHER; note = 2'-O-methyl modified nucleotide with phosphorodithioate linkage
  modified_base 99: mod_base = OTHER; note = 2'-O-methyl modified nucleotide with phosphorodithioate linkage